(19) United States
(12) Patent Application Publication
(10) Pub. No.: US 2025/0360143 A1
(43) Pub. Date: Nov. 27, 2025

(54) COMPOSITIONS AND METHODS FOR PHARMACOLOGIC TREATMENT OF STROKE AND MYOCARDIAL ISCHEMIA REPERFUSION INJURY

(71) Applicants: University of Connecticut, Farmington, CT (US); The University of America, as represented by the Secretary, Department of Health and Human Services, Bethesda, MD (US)

(72) Inventors: Bruce T. Liang, Avon, CT (US); Rajkumar Verma, Woodbridge, CT (US); Kenneth A. Jacobson, Silver Spring, MD (US); Kiran S. Toti, Atlanta, GA (US)

(21) Appl. No.: 18/872,564

(22) PCT Filed: Jun. 7, 2023
(86) PCT No.: PCT/US2023/024676

§ 371 Date: Dec. 6, 2024

Related U.S. Application Data
(60) Provisional application No. 63/349,626, filed on Jun. 7, 2022.

(51) Int. Cl. A61K 31/5513; A61P 9/10

(52) U.S. Cl. CPC A61K 31/5513; A61P 9/10

(57) ABSTRACT

Compositions and methods for the treatment of a human subject who has had a stroke or myocardial ischemia reperfusion injury, by administering to the subject a pharmaceutical composition including a compound of Formula (I) and/or of Formula (5) or a pharmaceutically acceptable salt and/or formulation thereof. The pharmaceutical composition can be administered in the acute phase of stroke, optionally in combination with a thrombolytic therapeutic or a procedure on the subject involving a clot-removal device.

### CROSS-REFERENCE TO RELATED APPLICATION

This application claims priority under 35 U.S.C. § 119(e) to U.S. Provisional Patent Application Ser. No. 63/349,626, filed on Jun. 7, 2022, the contents of which are hereby incorporated by reference in their entirety.

### STATEMENT REGARDING FEDERALLY SPONSORED RESEARCH & DEVELOPMENT

This invention was made with government support under HL156322, and DK031127 awarded by the National Institutes of Health. The U.S. Government has certain rights in the invention.

### BACKGROUND

### 1. Field of the Invention

Disclosed herein are compositions and methods for the treatment of stroke, specifically ischemic stroke, or reperfusion injury resulting from treatment of myocardial infarction.

### 2. Description of the Related Art

Stroke remains one of the leading causes of death and disability in the United States. For example, stroke is the fifth leading cause of death, as more than 140,000 people die each year from stroke in the United States. Stroke is the second leading cause of disability in Europe after ischemic heart disease (IHD) and is the sixth leading cause worldwide. The prevalence of stroke events in the United States has been project to increase due to an aging population, with an addition 3.4 million people suffering a stroke in 2030 relative to 2012.

The majority of strokes are ischemic strokes, in which blood flow to the brain is disrupted. Despite recent advances, interventions to reduce damage and enhance recovery after stroke are lacking. For example, current approaches to treating ischemic stroke include administration of thrombolytic therapeutics such as tissue plasminogen activator, and an invasive endovascular procedure using a clot removing/retrieving device. Thrombolytic therapeutics, however, must be given during the first few hours of a stroke, and are associated with a risk of bleeding. The clot removing/retrieving device is useful in less than 10% of embolic stroke cases. While the current thrombolytic alteplase and endovascular thrombectomy have increased reperfusion and improved clinical outcomes, nearly half of ischemic stroke patients do not recover to achieve an independent lifestyle. Mortality and morbidity remain high after ischemic stroke, indicating a very high unmet medical need, necessitating novel approaches.

Myocardial infarction (MI) (i.e., heart attack) is the irreversible death (necrosis and apoptosis) of heart muscle secondary to prolonged lack of oxygen supply (ischemia). Approximately 1.5 million cases of MI occur annually in the United States. Examples of myocardial infarction include ST elevation myocardial infarction (STEMI), Non-ST elevation myocardial infarction (NSTEMI) & acute myocardial infarction. Reperfusion following ischemia results in an influx of circulating immune cells, such as neutrophils and monocytes, to the injured myocardium. While ischemia caused by occlusion of the coronary artery leads to infarcted myocardium, reopening of the blocked artery may further contribute significantly to cardiac injury known as myocardial ischemia reperfusion injury. Clinically in patients, such reperfusion injury occurs after opening of the blocked coronary artery via percutaneous coronary intervention with a stent or thrombolytic medication. Thus, preventing myocardial ischemia reperfusion injury may reduce infarct size or prevent deterioration of cardiac function.

Thus, improved pharmacological therapy for stroke, particularly ischemic stroke, and myocardial ischemia reperfusion injury represent areas of unmet need in the art.

### BRIEF SUMMARY

In an aspect, a method for treatment of a human subject who has had a stroke or myocardial ischemia reperfusion injury includes administering to the subject a pharmaceutical composition comprising a compound of Formula (I), or a pharmaceutically acceptable salt and/or formulation thereof, the method including:
administering to the subject a pharmaceutical composition comprising a compound of Formula (I)

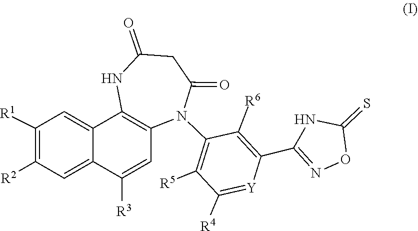

or a pharmaceutically acceptable salt thereof, wherein in Formula (I),
R¹ is hydrogen, cyano, halo, nitro, C₁-C₃ alkyl, or C₁-C₃ haloalkyl,
R² is hydrogen, cyano, halo, nitro, C₁-C₆ alkyl, C₁-C₆ haloalkyl, C₁-C₆ alkoxy, C₁-C₆ haloalkoxy, C₃-C cycloalkyl, C₃-C₈ cycloalkoxy, C₂-C₇ heterocycloalkyl, C₆-C₁₂ aryl, C₂-C₁₁ heteroaryl, C₂-C₆ alkanoyl, —COOH, —NR^{a}R^{b}, —C(O)—OR^{c}, —C(O)—NR^{a}R^{b}, —SO₂—OR^{c} or —SO₂—NR^{a}R^{b}, wherein R^{a}, R^{b}, and R^{c} are independently hydrogen, C₁-C₆ alkyl, or C₁-C₆ haloalkyl,
R³ is hydrogen, cyano, halo, nitro, C₁-C₆ alkyl, C₁-C₆ haloalkyl, C₁-C₆ alkoxy, C₁-C₆ haloalkoxy, C₃-C₈ cycloalkyl, C₃-C₈ cycloalkoxy, C₂-C₇ heterocycloalkyl, C₆-C₁₂ aryl, C₂-C₁₁ heteroaryl, C₂-C₆ alkanoyl, —COOH, —NR^{a}R^{b}, —C(O)—OR^{a}, —C(O)—NR^{a}R^{b}, —SO₂—OR^{a} or —SO₂—NR^{a}R^{b}, wherein R^{a} and R^{b}, and R⁶ are each independently hydrogen, C₁-C₆ alkyl, or C₁-C₆ haloalkyl,
R⁴, R⁵, and R⁶ are each independently hydrogen, cyano, halo, nitro, C₁-C₆ alkyl, C₁-C₆ haloalkyl, C₁-C₆ alkoxy, C₁-C₆ haloalkoxy, C₃-C₈ cycloalkyl, C₃-C₈ cycloalkoxy, C₂-C₇ heterocycloalkyl, C₆-C₁₂ aryl, C₂-C₁₁ heteroaryl, C₂-C₆ alkanoyl, —COOH, —NR^{a}R^{b}, —C(O)—OR^{c}, —C(O)—NR^{a}R^{b}, —SO₂—OR^{c} or —SO₂—NR^{a}R^{c},
with the proviso that not all of R¹, R², and R³ are hydrogen; and
Y is CH, CR⁴, or N.

In another aspect, the compounds of Formula (I), in particular Formulas (Ia), (Ib), or (Ic), or a pharmaceutically acceptable salt and/or formulation thereof are disclosed.

In another aspect, a method for treatment of a human subject who has had a stroke or myocardial ischemia reperfusion injury includes administering to the subject a pharmaceutical composition comprising a compound of Formula (5)

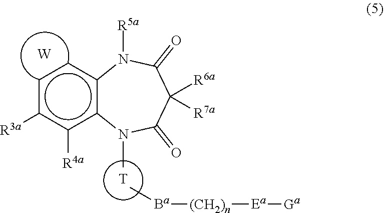

or a pharmaceutically acceptable salt thereof, wherein, in Formula (5),
   the moiety

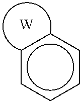

is naphthalene ring, quinoline ring, isoquinoline ring, tetrahydronaphthalene ring, indane ring, tetrahydroquinoline ring, or tetrahydroisoquinoline ring, each ring optionally substituted with 1 to 4 substituents that are the same or different and are C₁₋₈ alkyl, C₂₋₈ alkenyl, C₁₋₈ alkoxy, C₁₋₈ alkyl substituted with 1 to 3 halogen atoms, C₁₋₈ alkoxy substituted with 1 to 3 halogen atoms, a halogen atom, hydroxyl, nitro, cyano, amino, C₁₋₈ alkylamino, C₂₋₈ dialkylamino, C₂₋₈ acylamino, carboxyl, C₂₋₈ acyl, an alkoxycarbonyl (wherein the alkoxy moiety has 1 to 8 carbon atoms), or an aralkyl (wherein the aryl moiety has 6 to 10 carbon atoms, and the alkylene moiety has 1 to 8 carbon atoms),
R^{3a} and R^{4b} are the same or different, and are a hydrogen atom, C₁₋₈ alkyl, C₂₋₈ alkenyl, C₁₋₈ alkoxy, C₁₋₈ alkyl substituted with 1 to 3 halogen atoms, C₁₋₈ alkoxy substituted with 1 to 3 halogen atoms, a halogen atom, hydroxyl, nitro, cyano, amino, C₁₋₈ alkylamino, C₂₋₈ dialkylamino, C₂₋₈ acylamino, carboxyl, C₂₋₈ acyl, an alkoxycarbonyl group (wherein the alkoxy moiety has 1 to 8 carbon atoms), or an aralkyl group (wherein the aryl moiety has 6 to 10 carbon atoms, and the alkylene moiety has 1 to 8 carbon atoms),
R^{5a} is a hydrogen atom, C₁₋₈ alkyl, C₂₋₈ alkenyl, C₁₋₈ alkyl substituted with 1 to 3 halogen atoms, C₁₋₈ alkyl substituted with a hydroxyl, or an aralkyl (wherein the aryl moiety has 6 to 10 carbon atoms, and the alkylene moiety has 1 to 8 carbon atoms),
R^{6a} and R^{7a} are the same or different, and represent a hydrogen atom, C₁₋₈ alkyl, C₂₋₈ alkenyl, C₁₋₈ alkoxy, C₁₋₈ alkyl substituted with 1 to 3 halogen atoms, C₁₋₈ alkoxy substituted with 1 to 3 halogen atoms, a halogen atom, hydroxyl, or amino,
the moiety

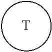

is a benzene ring, pyridine ring, thiophene ring, pyrimidine ring, naphthalene ring, quinoline ring, or indole ring, which can optionally have 1 to 4 of the same or different substituents that can be C₁₋₈ alkyl, C₂₋₈ alkenyl, C₁₋₈ alkoxy, C₁₋₈ alkyl substituted with 1 to 3 halogen atoms, C₁₋₈ alkoxy substituted with 1 to 3 halogen atoms, a halogen atom, hydroxyl, nitro, cyano, amino, C₁₋₈ alkylamino, C₂₋₈ dialkylamino, an aralkyl group (wherein the aryl moiety has 6 to 10 carbon atoms, and the alkylene moiety has 1 to 8 carbon atoms), phenyl, or pyridyl, as a substituent,
B^{a} is N(R^{8a})C(═O), NHCONH, CON(R^{9a}), NHC(═S)NH, N(R^{10a})SO₂, SO₂N(R^{11a}), or OSO₂, wherein R^{8a}, R^{9a}, R^{10a} and R¹¹ are hydrogen, C₁₋₈ alkyl, C₁₋₈ alkyl substituted with 1 to 3 halogen atoms, C₁₋₈ alkyl substituted with a hydroxyl, or an aralkyl wherein the aryl moiety has 6 to 10 carbon atoms, and the alkylene moiety has 1 to 8 carbon atoms,
E^{a} is O, S, NR^{12a}, or an atomic bond, wherein R^{12a} is a hydrogen, C₁₋₈ alkyl, C₂₋₈ alkenyl, a C₁-8 alkyl substituted with 1 to 3 halogen atoms, C₁₋₈ alkyl substituted with a hydroxyl, or an aralkyl (wherein the aryl moiety has 6 to 10 carbon atoms, and the alkylene moiety has 1 to 8 carbon atoms),
G^{a} is piperazine, piperidine, morpholine, cyclohexane, benzene, naphthalene, quinoline, quinoxaline, benzimidazole, thiophene, imidazole, thiazole, oxazole, indole, benzofuran, pyrrole, pyridine, or pyrimidine, which can be optionally substituted with 1 to 4 of the same or different substituents that can be C₁₋₈ alkyl, C₂₋₈ alkenyl, C₁₋₈ alkoxy, C₁₋₈ alkyl substituted with 1 to 3 halogen atoms, C₁₋₈ alkoxy substituted with 1 to 3 halogen atoms, a halogen atom, hydroxyl, nitro, cyano, amino, C₁₋₈ alkylamino, C₂₋₈ dialkylamino, C₂₋₈ acyl, methylenedioxy, carboxyl, C₁₋₆ alkylsulfinyl, C₁₋₆ alkylthio, C₁₋₆ alkylsulfonyl, an aralkyl (wherein the aryl moiety has 6 to 10 carbon atoms, and the alkylene moiety has 1 to 8 carbon atoms), an optionally substituted phenyl, an optionally substituted pyridyl, an optionally substituted imidazolyl, an optionally substituted oxazolyl, or an optionally substituted thiazolyl, as a substituent, and
n is an integer of 0 to 5.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the dose-dependent effect of MRS 4719 on ischemic stroke infarct size, in particular the results of a three-day treatment with MRS 4719 (0.5-3 mg/kg/day for 3 days continuous infusion with alzet minipump), where a representative TTC stained section showing infarct area (dotted line) is shown in the upper portion of FIG. A, and a graph of location in the brain versus infarct volume (% contralateral) is shown in the lower portion.

FIG. 2 shows the effect of MRS 4596 post-treatment on stroke infarct size, in particular the results of a three-day post-treatment with MRS 4596 (5.0 mg/kg/day for 3 days continuous infusion with alzet minipump), where a representative TTC stained section showing infarct area (dotted line) in upper portion of FIG. B and a graph of a graph of location in the brain versus infarct volume (% contralateral) is shown in the lower portion. Three-day post-treatment with MRS 4596 (5.0 mg/kg/day×3 days continuous infusion with alzet minipump) significantly (*p<0.05; vs Veh) reduced total hemispheric infarct volume (n=5-7/treatment group). Data are presented as Mean±S.D.

FIG. 3 shows the effect of MRS 4719 treatment on learning and memory in middle-aged mice after stroke and reached to statistical significance at dose 3 mg/kg, (*p<0.05 vs Veh, One-way anova followed by Tukey's multiple comparison test) measured by Novel Object Recognition Test. (n=5-11/treatment group=32 mice total; 19 males and 13 females). Data are Mean±S.D.

FIG. 4 shows the effect of duration of MRS 4719 (1.5 mg/kg body weight (b.w.)) treatment sensorimotor task in middle aged mice after stroke. Short term treatment for two days improved sensorimotor during progressive recovery as shown by increase in latency to fall time (*p<0.05 vs Veh, one-way anova followed by Tukey's multiple comparison test) measured by Novel Object Recognition Test. (n=5-15/treatment group=50 mice total; 28 males and 22 females). Data are Mean±S.D., where MCAo is middle cerebral artery occlusion; DT is days for treatment.

FIG. 5 shows the effects of duration of MRS 4719 (1.5 mg/kg b.w.) treatment on learning and memory in middle aged mice after stroke. Similar to sensorimotor task, short term treatment for two days improved memory and learning as shown by increase in discrimination index (*p<0.05 vs Veh,0One-way anova followed by Tukey's multiple comparison test) measured by Novel Object Recognition Test. (n=5-15/treatment group=50 mice total; 28 males and 22 females). Data are Mean±S.D.

FIG. 6A and FIG. 6B show the effect of systemic infusion of MRS 4719, where wild type BL6 mice, 10-12 weeks, were subjected to left anterior descending artery (LAD) ligation with infusion of MRS 4719 at reperfusion. Data is shown at 2 weeks (FIG. 6A) or at 4 weeks (FIG. 6B). Data were mean and SEM. Decreases in EF and FS in MRS 4719 treated were less than those in vehicle-treated mice at 2 weeks or 4 weeks post-I/R (P<0.05, t test).

The above-described and other features will be appreciated and understood by those skilled in the art from the following detailed description, examples, and claims.

### DETAILED DESCRIPTION

P2X4R is a receptor for adenosine triphosphate (ATP) and is an important neurotransmitter receptor in the brain. It regulates activation of myeloid immune cells (infiltrating monocytes/macrophages and brain-resident microglia) after stroke injury. However, over-stimulation of P2X4Rs due to excessive ATP release from dying or damaged neuronal cells can contribute to ischemic injury. Reducing immune inflammation arising from over-stimulation of P2X4Rs would be useful in the treatment of stroke.

In an aspect, blocking pro-inflammatory P2X4R during myocardial ischemia reperfusion is beneficial, resulting in reduced injury, infarct size, restoration of cardiac performance toward normal, and the like.

A number of P2X4R antagonists have been developed over the years for other uses, for example treating multiple sclerosis and reducing chronic neuropathic pain in vivo, as well as stroke protection. Representative antagonists for this receptor are shown below.

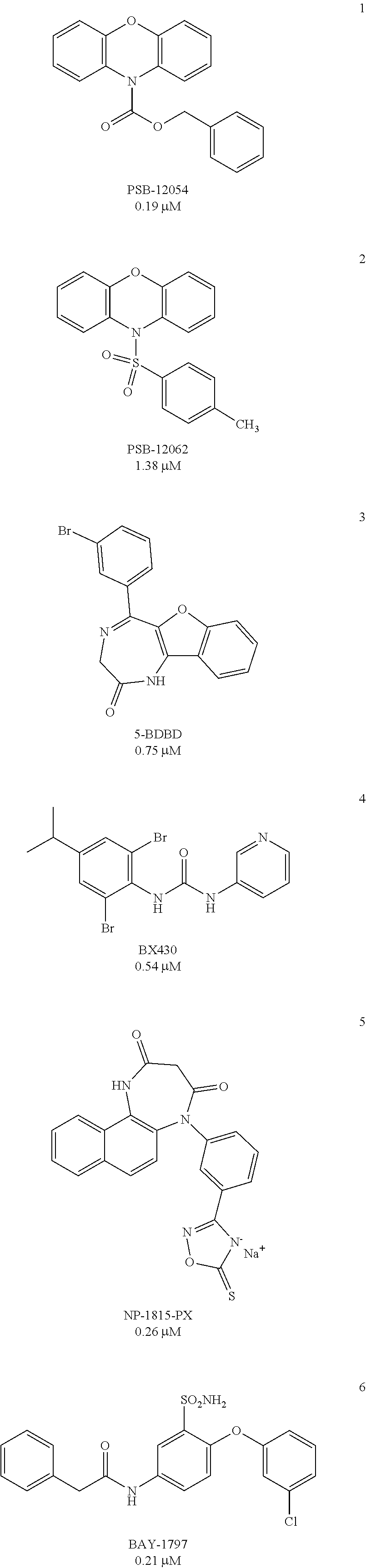

Seven P2X subunits form functional trimeric cation channels, and heterotrimeric channels can differ in ligand activity and other pharmacological properties from homotrimeric channels, which contributes to the difficulty in developing effective antagonists for use in the treatment of stroke. In addition, it is desirable for the antagonist to be selective for P2X4R. The inventors hereof have investigated compounds and methods to pharmacologically inhibit P2X4R, to limit the over-stimulated myeloid cell immune response and improve both acute and chronic stroke recovery. The compounds and methods can be used alone, or as an adjunct therapy concomitant with thrombolytic therapeutics, clot retrieval, other treatments, or a combination thereof. The P2X4R antagonists investigated are based on substituted 1,5-dihydro-2H-naphtho[1,2-b][1,4]diazepine-2,4(3H)-diones having the basic structure (II)

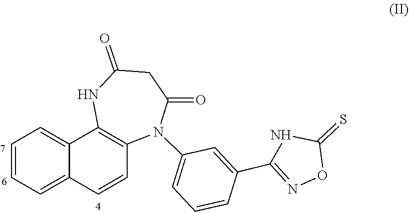 [SMILES: O=C1CC(=O)N(c2cccc(-c3noc(=S)[nH]3)c2)c2ccc3ccccc3c2N1]

where positions 4, 6, and 7 at the naphthalene ring are numbered for convenience. The sodium salt of this structure is also known as NP-1815-PX (compound 5 above) and has been reported to have an IC₅₀ value at the human P2X4R (hP2X4R) of 0.26 μM.

In particular, a series of compounds and pharmaceutically acceptable salts and/or formulations thereof were investigated, having various substituents at positions 4, 6, and 7 of the naphthalene ring and one or more heteroatoms replacing carbon in the phenyl ring. Specific compounds tested are described in the Examples. The inventors hereof have discovered that compounds of Formula (I)

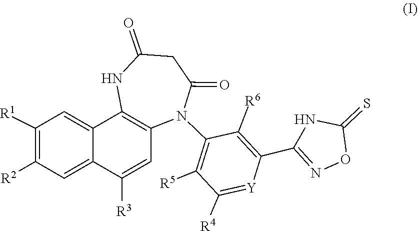

and pharmaceutically acceptable salts and/or formulations thereof are antagonists of hP2X4R. In the compounds of Formula (I),
   R¹ is hydrogen, cyano, halo, nitro, C₁-C₃ alkyl, or C₁-C₃ haloalkyl,
   R² is hydrogen, cyano, halo, nitro, C₁-C₆ alkyl, C₁-C₆ haloalkyl, C₁-C₆ alkoxy, C₁-C₆ haloalkoxy, C₃—C cycloalkyl, C₃-C₈ cycloalkoxy, C₂-C₇ heterocycloalkyl, C₆-C₁₂ aryl, C₂-C₁₁ heteroaryl, C₂-C₆ alkanoyl, —COOH, —NR^{a}R^{b}, —C(O)—OR^{c}, —C(O)—NR^{a}R^{b}, —SO₂—OR^{c} or —SO₂—NR^{a}R^{b}, wherein R^{a}, R^{b}, and R^{c} are independently hydrogen, C₁-C₆ alkyl, or C₁-C₆ haloalkyl,
   R³ is hydrogen, cyano, halo, nitro, C₁-C₆ alkyl, C₁-C₆ haloalkyl, C₁-C₆ alkoxy, C₁-C₆ haloalkoxy, C₃-C₈ cycloalkyl, C₃-C₈ cycloalkoxy, C₂-C₇ heterocycloalkyl, C₆-C₁₂ aryl, C₂-C₁₁ heteroaryl, C₂-C₆ alkanoyl, —COOH, —NR^{a}R^{b}, —C(O)—OR^{a}, —C(O)—NR^{a}R^{b}, —SO₂—OR^{a} or —SO₂—NR^{a}R^{b}, wherein R^{a} and R^{b}, and R⁶ are each independently hydrogen, C₁-C₆ alkyl, or C₁-C₆ haloalkyl,
   R⁴, R⁵, and R⁶ are each independently hydrogen, cyano, halo, nitro, C₁-C₆ alkyl, C₁-C₆ haloalkyl, C₁-C₆ alkoxy, C₁-C₆ haloalkoxy, C₃-C₈ cycloalkyl, C₃-C₈ cycloalkoxy, C₂-C₇ heterocycloalkyl, C₆-C₁₂ aryl, C₂-C₁₁ heteroaryl, C₂-C₆ alkanoyl, —COOH, —NR^{a}R^{b}, —C(O)—OR^{c}, —C(O)—NR^{a}R^{b}, —SO₂—OR^{c} or —SO₂—NR^{a}R^{c},
with the proviso that not all of R¹, R², and R³ are hydrogen; and
Y is CH, CR⁴, or N.

In an aspect, in the compounds or pharmaceutically acceptable salts of Formula (I),
R¹ is hydrogen, cyano, halo, methyl, or halomethyl,
R² is hydrogen, cyano, halo, C₁-C₆ alkyl, C₁-C₆ haloalkyl, C₁-C₆ alkoxy, C₁-C₆ haloalkoxy, C₃-C₆ cycloalkyl, C₃-C₆ cycloalkoxy, C₂-C₅ heterocycloalkyl, C₆ aryl, C₂-C₆ heteroaryl, C₂-C₆ alkanoyl, —COOH, —NR^{a}R^{b}, —C(O)—OR^{a}, —C(O)—NR^{b}R⁶, —SO₂—OR⁷ or —SO₂—NR^{b}R⁶, wherein R^{a}, R^{b}, and R⁶ are each independently hydrogen, C₁-C₃ alkyl, or C₁-C₃ haloalkyl,
R³ is hydrogen, C₁-C₆ alkyl, C₁-C₆ haloalkyl, C₁-C₆ alkoxy, C₁-C₆ haloalkoxy, C₃-C₆ cycloalkyl, C₃-C₆ cycloalkoxy, C₂-C₈ heterocycloalkyl, C₆ aryl, C₂-C₆ heteroaryl, C₂-C₆ alkanoyl, —COOH, —NR^{a}R^{b}, —C(O)—OR^{c}, —C(O)—NR^{a}R^{b}, —SO₂—OR^{c} or —SO₂—NR^{a}R^{b}, wherein R^{a}, R^{b}, and R^{c} are each independently hydrogen, C₁-C₃ alkyl, or C₁-C₃ haloalkyl,
R⁴, R⁵, and R⁶ are each independently hydrogen, C₁-C₆ alkyl, C₁-C₆ haloalkyl, C₁-C₆ alkoxy, C₁-C₆ haloalkoxy, C₃-C₆ cycloalkyl, C₃-C₆ cycloalkoxy, C₂-C₅ heterocycloalkyl, C₆ aryl, C₂-C₆ heteroaryl, C₂-C₆ alkanoyl, —COOH, —NR^{a}R^{b}, —C(O)—OR^{c}, —C(O)—NR^{a}R^{b}, —SO₂—OR^{c} or —SO₂—NR^{a}R^{b},
with the proviso that not all of R¹, R², and R³ are hydrogen; and
Y is CH, CR⁴, or N.

In another aspect, in the compounds or pharmaceutically acceptable salts of Formula (I),
R¹ is hydrogen, cyano, halo, methyl, or halomethyl,
R² is hydrogen, cyano, halo, C₁-C₃ alkyl, C₁-C₃ haloalkyl, C₁-C₃ alkoxy, C₁-C₃ haloalkoxy, C₂-C₃ alkanoyl, —COOH, —NR^{a}R^{b}, —C(O)—OR^{a}, —C(O)—NR^{a}R^{b}, —SO₂—OR or —SO₂—NR^{a}R^{b}, wherein R^{a}, R^{b}, and R^{c} are independently hydrogen, C₁-C₃ alkyl, or C₁-C₃ haloalkyl,
R³ is hydrogen, cyano, halo, C₁-C₃ alkyl, C₁-C₃ haloalkyl, C₁-C₃ alkoxy, C₁-C₃ haloalkoxy, C₂-C₃ alkanoyl, —COOH, —NR^{a}R^{b}, —C(O)—OR^{c}, —C(O)—NR^{a}R^{b}, —SO₂—OR^{c} or —SO₂—NR^{a}R^{b}, wherein R^{a}, R^{b}, and R^{c} are each independently hydrogen, C₁-C₃ alkyl, or C₁-C₃ haloalkyl,
R⁴, R⁵, and R⁶ are each independently hydrogen, cyano, halo, C₁-C₃ alkyl, C₁-C₃ haloalkyl, C₁-C₃ alkoxy, C₁-C₃ haloalkoxy, C₂-C₃ alkanoyl, —COOH, —NR^{a}R^{b}, —C(O)—OR^{c}, —C(O)—NR^{a}R^{b}, —SO₂—OR^{c} or —SO₂—NR^{a}R^{b},
with the proviso that not all of R¹, R², and R³ are hydrogen; and
Y is CH, CR⁴, or N.

In still another aspect, in the compounds or pharmaceutically acceptable salts of Formula (I),
R¹ is hydrogen,
R² is hydrogen, cyano, halo, C₁-C₃ alkyl, C₁-C₃ haloalkyl, C₁-C₃ alkoxy, C₁-C₃ haloalkoxy, C₂-C₃ alkanoyl, —NR^{a}R^{b}, —C(O)—OR^{c}, —C(O)—NR^{a}R^{b}, —SO₂—OR^{c} or —SO₂—NR^{a}R^{b}, wherein R^{a}, R^{b}, and R^{c} are each independently hydrogen, C₁-C₃ alkyl, or C₁-C₃ haloalkyl,
R³ is hydrogen, cyano, halo, C₁-C₃ alkyl, C₁-C₃ haloalkyl, C₁-C₃ alkoxy, C₁-C₃ haloalkoxy, C₂-C₃ alkanoyl, —COOH, —NR^{a}R^{b}, —C(O)—OR^{c}, —C(O)—NR^{a}R^{b}, —SO₂—OR^{c} or —SO₂—NR^{a}R^{b}, wherein R^{a}, R^{b}, and R^{c} are each independently hydrogen, C₁-C₃ alkyl, or C₁-C₃ haloalkyl,
R⁴, R⁵, and R⁶ are each independently hydrogen, cyano, halo, C₁-C₃ alkyl, C₁-C₃ haloalkyl, C₁-C₃ alkoxy, C₁-C₃ haloalkoxy, C₂-C₃ alkanoyl, —COOH, —NR^{a}R^{b}, —C(O)—OR^{c}, —C(O)—NR^{a}R^{b}, —SO₂—OR^{c} or —SO₂—NR^{a}R^{b},
with the proviso that not all of R¹, R², and R³ are hydrogen; and
Y is CH, CR⁴, or N.

In yet another aspect, in the compounds or pharmaceutically acceptable salts of Formula (I),
R¹ is hydrogen,
R² is hydrogen, cyano, halo, C₁-C₃ alkyl, C₁-C₃ haloalkyl, C₁-C₃ alkoxy, or C₁-C₃ haloalkoxy,
R³ is hydrogen, cyano, halo, C₁-C₃ alkyl, C₁-C₃ haloalkyl, C₁-C₃ alkoxy, or C₁-C₃ haloalkoxy,
R⁴, R⁵, and R⁶ are each independently hydrogen, cyano, halo, C₁-C₃ alkyl, C₁-C₃ haloalkyl, C₁-C₃ alkoxy, or C₁-C₃ haloalkoxy,
with the proviso that not all of R¹, R², and R³ are hydrogen; and
Y is CH, CR⁴, or N.

Preferably in this aspect, R⁴ is cyano, halo, C₁-C₃ alkyl, C₁-C₃ haloalkyl, C₁-C₃ alkoxy, or C₁-C₃ haloalkoxy, and R⁵ and R⁶ are each hydrogen.

In a preferred aspect, in the compounds or pharmaceutically acceptable salts of Formula (I),
R¹ is hydrogen,
R² is hydrogen, halo, or C₁-C₃ alkyl,
R³ is hydrogen, halo, or C₁-C₃ alkyl,
R⁴ is hydrogen, halo, or C₁-C₃ alkyl,
R⁵ and R⁶ are each hydrogen,
with the proviso that not all of R¹, R², and R³ are hydrogen; and
Y is CH, CR⁴, or N.

In another preferred aspect, the compound of Formula (I) or the pharmaceutically acceptable salt thereof is a compound of the following formulas. In a specific aspect the compounds are of the salts indicated in Table 1.

TABLE 1

| Compound | Structure | Salt form |
|---|---|---|
| | 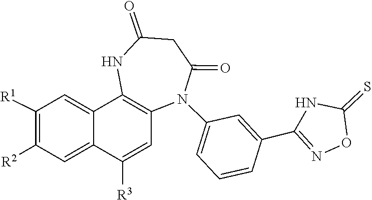 | |
| 22b | R¹ = CH₃, R² = R³ = H | Sodium salt |
| 22c^{d} | R¹ = H, R² = CH3, R³ = H | Sodium salt |
| 21d^{d} | R¹ = R² = H, R³ = Br | Triethylamine salt |
| 21e | R¹ = CH₃, R² = H, R³ = Br | Triethylamine salt |
| 21j | R¹ = H, R² = CH₂N₃, R³ = H | Triethylamine salt |
| 211 | R¹ = R² = H, R³ = Ph | Triethylamine salt |
| 21p | R¹ = R² = H, R³ = Et | Triethylamine salt |
| | 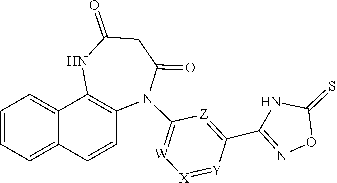 | |
| 21r | X = CCH₃, W = Y = Z = CH | Triethylamine salt |
| 21s | W = N, X = Y = Z = CH | Triethylamine salt |
| 21t | X = N, W = Y = Z = CH | Triethylamine salt |
| 21u^{d} | Y = N, W = X = Z = CH | Triethylamine salt |
| 21v | Z = N, W = X = Y = CH | Triethylamine salt |

Preferred compounds of Formula (I) are compounds or pharmaceutically acceptable salts of chemical formulas (Ia), (Ib), or (Ic).

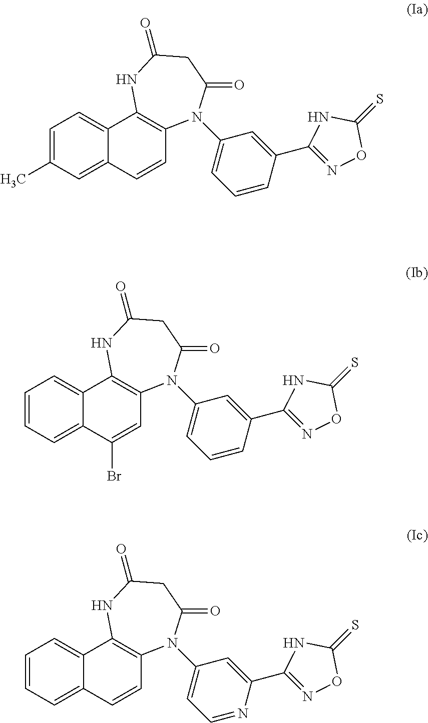 [SMILES: Cc1ccc2c3c(ccc2c1)N(c1cccc(-c2noc(=S)[nH]2)c1)C(=O)CC(=O)N3 ; O=C1CC(=O)N(c2cccc(-c3noc(=S)[nH]3)c2)c2cc(Br)c3ccccc3c2N1 ; O=C1CC(=O)N(c2ccnc(-c3noc(=S)[nH]3)c2)c2ccc3ccccc3c2N1]

The compounds of chemical formulas (Ia), (Ib), or (Ic) are also referred to in the Examples as compounds 21c, 21d, and 21u, respectively.

Other compounds that can be used in the for the treatment of stroke, specifically ischemic stroke, or reperfusion injury resulting from treatment of myocardial infarction in a mammal such as a human have been reported in U.S. Pat. No. 11,434,207 issued on Sep. 6, 2022 to Ushioda et al., titled “P2X4 Receptor Agonist”, which is hereby incorporated by reference in its entirety. In an aspect, these compounds are of Formula (5), or a pharmacologically acceptable salt thereof:

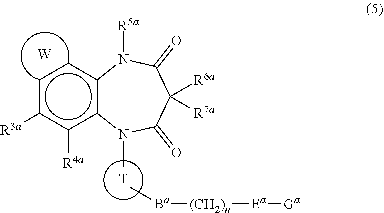

wherein, in Formula (5), the moiety

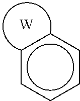

is naphthalene ring, quinoline ring, isoquinoline ring, tetrahydronaphthalene ring, indane ring, tetrahydroquinoline ring, or tetrahydroisoquinoline ring, wherein these rings are optionally substituted with 1 to 4 of the same or different substituents that are C₁₋₈ alkyl, C₂₋₈ alkenyl, C₁₋₈ alkoxy, C₁₋₈ alkyl substituted with 1 to 3 halogen atoms, C₁₋₈ alkoxy substituted with 1 to 3 halogen atoms, a halogen atom, hydroxyl, nitro, cyano, amino, C₁₋₈ alkylamino, C₂₋₈ dialkylamino, C₂₋₈ acylamino, carboxyl, C₂₋₈ acyl, an alkoxycarbonyl (wherein the alkoxy moiety has 1 to 8 carbon atoms), or an aralkyl (wherein the aryl moiety has 6 to 10 carbon atoms, and the alkylene moiety has 1 to 8 carbon atoms),
R^{3a} and R^{4b} are the same or different, and are a hydrogen, C₁₋₈ alkyl, C₂₋₈ alkenyl, C₁₋₈ alkoxy, C₁₋₈ alkyl substituted with 1 to 3 halogen atoms, C₁₋₈ alkoxy substituted with 1 to 3 halogen atoms, a halogen atom, hydroxyl, nitro, cyano, amino, C₁₋₈ alkylamino, C₂₋₈ dialkylamino, C₂₋₈ acylamino, carboxyl, C₂₋₈ acyl, an alkoxycarbonyl (wherein the alkoxy moiety has 1 to 8 carbon atoms), or an aralkyl (wherein the aryl moiety has 6 to 10 carbon atoms, and the alkylene moiety has 1 to 8 carbon atoms),
R^{5a} is a hydrogen, C₁₋₈ alkyl, C₂₋₈ alkenyl, C₁₋₈ alkyl substituted with 1 to 3 halogen atoms, C₁₋₈ alkyl substituted with hydroxyl, or an aralkyl group (wherein the aryl moiety has 6 to 10 carbon atoms, and the alkylene moiety has 1 to 8 carbon atoms),
R^{6a} and R^{7a} are the same or different, and are a hydrogen, C₁₋₈ alkyl, C₂₋₈ alkenyl, C₁₋₈ alkoxy, C₁₋₈ alkyl substituted with 1 to 3 halogen atoms, C₁₋₈ alkoxy substituted with 1 to 3 halogen atoms, a halogen atom, hydroxyl, or amino,

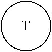

the moiety is a benzene ring, pyridine ring, thiophene ring, pyrimidine ring, naphthalene ring, quinoline ring, or indole ring, which can optionally have 1 to 4 of the same or different substituents that can be C₁₋₈ alkyl, C₂₋₈ alkenyl, C₁₋₈ alkoxy, C₁₋₈ alkyl substituted with 1 to 3 halogen atoms, C₁₋₈ alkoxy substituted with 1 to 3 halogen atoms, a halogen atom, hydroxyl, nitro, cyano, amino, C₁₋₈ alkylamino, C₂₋₈ dialkylamino, an aralkyl (wherein the aryl moiety has 6 to 10 carbon atoms, and the alkylene moiety has 1 to 8 carbon atoms), phenyl, and pyridyl, as a substituent,
B^{a} is N(R^{8a})C(═O), NHCONH, CON(R^{9a}), NHC(═S)NH, N(R^{10a})SO₂, SO₂N(R^{11a}), or OSO₂, wherein R^{8a}, R^{9a}, R^{10a} and R^{11a} are a hydrogen, C₁₋₈ alkyl, C₁₋₈ alkyl substituted with 1 to 3 halogen atoms, C₁₋₈ alkyl substituted with a hydroxyl, or an aralkyl (wherein the aryl moiety has 6 to 10 carbon atoms, and the alkylene moiety has 1 to 8 carbon atoms),
E^{a} is O, S, NR^{12a}, or an atomic bond, wherein R^{12a} is a hydrogen, C₁₋₈ alkyl, C₂₋₈ alkenyl, C₁₋₈ alkyl substituted with 1 to 3 halogen atoms, C₁₋₈ alkyl substituted with a hydroxyl, or an aralkyl group (wherein the aryl moiety has 6 to 10 carbon atoms, and the alkylene moiety has 1 to 8 carbon atoms),
G^{a} is piperazine, piperidine, morpholine, cyclohexane, benzene, naphthalene, quinoline, quinoxaline, benzimidazole, thiophene, imidazole, thiazole, oxazole, indole, benzofuran, pyrrole, pyridine, or pyrimidine, which can be optionally substituted with 1 to 4 of the same or different substituents that can be C₁₋₈ alkyl, C₂₋₈ alkenyl, C₁₋₈ alkoxy, C₁₋₈ alkyl substituted with 1 to 3 halogen atoms, C₁₋₈ alkoxy substituted with 1 to 3 halogen atoms, a halogen atom, hydroxyl, nitro, cyano, amino, C₁₋₈ alkylamino, C₂₋₈ dialkylamino, C₂₋₈ acyl, methylenedioxy, carboxyl, C₁₋₆ alkylsulfinyl, C₁₋₆ alkylthio, C₁₋₆ alkylsulfonyl, an aralkyl (wherein the aryl moiety has 6 to 10 carbon atoms, and the alkylene moiety has 1 to 8 carbon atoms), an optionally substituted phenyl, an optionally substituted pyridyl, an optionally substituted imidazolyl, an optionally substituted oxazolyl, or an optionally substituted thiazolyl, as a substituent, as a substituent, and
n is an integer of 0 to 5.

Specific compounds of Formula 5 include those wherein
the moiety

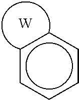

is a naphthalene ring or tetrahydronaphthalene ring, preferably a naphthalene ring, each of which is optionally substituted with 1 to 4 of the same or different groups, being C₁₋₈ alkyl, C₂₋₈ alkenyl, C₁₋₈ alkoxy, C₁₋₈ alkyl substituted with 1 to 3 halogen atoms, C₁₋₈ alkoxy substituted with 1 to 3 halogen atoms, a halogen atom, hydroxyl, nitro, cyano, amino, C₁₋₈ alkylamino, C₂₋₈ dialkylamino, C₂₋₈ acylamino, carboxyl, C₂₋₈ acyl, an alkoxycarbonyl (wherein the alkoxy moiety has 1 to 8 carbon atoms), or an aralkyl group (wherein the aryl moiety has 6 to 10 carbon atoms, and the alkylene moiety has 1 to 8 carbon atoms), as a substituent;
R^{3a} and R^{4a} may be the same or different, and are a hydrogen, C₁₋₈ alkyl, C₂₋₈ alkenyl, C₁₋₈ alkoxy, C₁₋₈ alkyl substituted with 1 to 3 halogen atoms, C₁₋₈ alkoxy substituted with 1 to 3 halogen atoms, a halogen atom, hydroxyl, nitro, cyano, amino, or an aralkyl (wherein the aryl moiety has 6 to 10 carbon atoms, and the alkylene moiety has 1 to 8 carbon atoms);
R^{6a} is a hydrogen, C₁₋₈ alkyl, or an aralkyl (wherein the aryl moiety has 6 to 10 carbon atoms, and the alkylene moiety has 1 to 8 carbon atoms), and is preferably a hydrogen;
R^{6a} and R^{7a} may be the same or different, and are a hydrogen, C₁₋₈ alkyl, C₁₋₈ alkoxy, C₁₋₈ alkyl substituted with 1 to 3 halogen atoms, or C₁₋₈ alkoxy substituted with 1 to 3 halogen atoms, preferably wherein both R^{6a} and R^{7a} are hydrogen atoms;

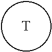

is a phenyl optionally substituted with 1 to 4 of the same or different substituents that can be C₁₋₈ alkyl, C₂₋₈ alkenyl, C₁₋₈ alkoxy, C₁₋₈ alkyl substituted with 1 to 3 halogen atoms, C₁₋₈ alkoxy substituted with 1 to 3 halogen atoms, a halogen atom, hydroxyl, nitro, cyano, amino, C₁₋₈ alkylamino, C₂₋₈ dialkylamino, an aralkyl (wherein the aryl moiety has 6 to 10 carbon atoms, and the alkylene moiety has 1 to 8 carbon atoms), phenyl, and pyridyl, as a substituent; preferably wherein

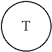

is phenyl with no substituents;
B^{a} is NHC(═O), NHCONH, CONH, NHC(═S)NH, NHSO₂, SO₂NH, or OSO; preferably wherein B^{a} is NHC(═O), NHCONH, or NHSO₂; and
Ea is an atomic bond;
G^{a} is piperazine, piperidine, morpholine, cyclohexane, benzene, naphthalene, quinoline, quinoxaline, benzimidazole, thiophene, imidazole, thiazole, oxazole, indole, benzofuran, pyrrole, pyridine, or pyrimidine, which is optionally substituted with 1 to 4 of the same or different substituents that are C₁₋₈ alkyl, C₂₋₈ alkenyl, C₁₋₈ alkoxy, C₁₋₈ alkyl substituted with 1 to 3 halogen atoms, C₁₋₈ alkoxy substituted with 1 to 3 halogen atoms, a halogen atom, hydroxyl, nitro, cyano, amino, C₁₋₈ alkylamino, C₂₋₈ dialkylamino, C₂₋₈ acyl, methylenedioxy, carboxyl, C₁₋₆ alkylsulfinyl, C₁₋₆ alkylthio, or C₁₋₆ alkylsulfonyl, as a substituent, preferably wherein G^{a} is benzene optionally substituted with 1 to 4 of the same or different substituents that are C₁₋₈ alkyl, C₂₋₈ alkenyl, C₁₋₈ alkoxy, C₁₋₈ alkyl substituted with 1 to 3 halogen atoms, C₁₋₈ alkoxy substituted with 1 to 3 halogen atoms, a halogen atom, hydroxyl, nitro, cyano, amino, C₁₋₈ alkylamino, C₂₋₈ dialkylamino, C₂₋₈ acyl, a methylenedioxy, a carboxyl, C₁₋₆ alkylsulfinyl, C₁₋₆ alkylthio, and C₁₋₆ alkylsulfonyl, as a substituent; and
n is 0.

In Formula 5, in a preferred aspect,

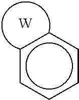

is a naphthalene ring and

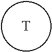

is a phenyl optionally substituted with 1 to 4 of the same or different substituents that can be C₁₋₈ alkyl, C₂₋₈ alkenyl, C₁₋₈ alkoxy, C₁₋₈ alkyl substituted with 1 to 3 halogen atoms, C₁₋₈ alkoxy substituted with 1 to 3 halogen atoms, a halogen atom, hydroxyl, nitro, cyano, amino, C₁₋₈ alkylamino, C₂₋₈ dialkylamino, an aralkyl (wherein the aryl moiety has 6 to 10 carbon atoms, and the alkylene moiety has 1 to 8 carbon atoms), phenyl, and pyridyl, as a substituent; or preferably wherein

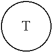

is phenyl with no substituents

Specific compounds of Formula (5) are listed in Table 2.

TABLE 2

| Abbr. | Compound Name |
|---|---|
| A | 1-[4-(2,4-dioxo-1,2,3,4-tetrahydronaphtho[1,2-b][1,4]diazepin-5-yl)phenyl]-3- |
| | phenylthiourea |
| B | 1-[4-(2,4-dioxo-1,2,3,4-tetrahydronaphtho[1,2-b][1,4]diazepin-5-yl)phenyl]-3- |
| | phenylurea |
| B | 1-(2-chlorophenyl)-3-[4-(2,4-dioxo-1,2,3,4-tetrahydronaphtho[1,2-b][1,4]diazepin-5- |
| | yl)phenyl]thiourea |
| C | 1-(2-chlorophenyl)-3-[4-(2,4-dioxo-1,2,3,4-tetrahydronaphtho[1,2-b][1,4]diazepin-5- |
| | yl)phenyl]urea |
| D | 1-[4-(2,4-dioxo-1,2,3,4-tetrahydronaphtho[1,2-b][1,4]diazepin-5-yl)phenyl]-3-(2- |
| | methylphenyl)thiourea |
| E | 5-(4-phenylcarbamoylphenyl)-1H-naphtho[1,2-b][1,4]diazepine-2,4(3H,5H)-dione |
| F | 5-(4-benzylcarbamoylphenyl)-1H-naphtho[1,2-b][1,4]diazepine-2,4(3H,5H)-dione |
| G | 5-[4-(2-chlorobenzyl)carbamoylphenyl]-1H-naphtho[1,2-b][1,4]diazepine-2,4(3H,5H)- |
| | dione |
| H | 5-[4-(2-chlorophenyl)carbamoylphenyl]-1H-naphtho[1,2-b][1,4]diazepine-2,4(3H,5H)- |
| | dione |
| I | N-[4-(2,4-dioxo-1,2,3,4-tetrahydronaphtho[1,2-b][1,4]diazepin-5-yl)phenyl]-2- |
| | nitrobenzenesulfonamide |
| J | N-[4-(2,4-dioxo-1,2,3,4-tetrahydronaphtho[1,2-b][1,4]diazepin-5- |
| | yl)phenyl]benzenesulfonamide |
| K | 3-bromo-N-[4-(2,4-dioxo-1,2,3,4-tetrahydronaphtho[1,2-b][1,4]diazepin-5- |
| | yl)phenyl]benzenesulfonamide |
| L | N-[4-(2,4-dioxo-1,2,3,4-tetrahydronaphtho[1,2-b][1,4]diazepin-5-yl)phenyl]-3- |
| | methoxybenzenesulfonamide |
| M | N-[3-(2,4-dioxo-1,2,3,4-tetrahydronaphtho[1,2-b][1,4]diazepin-5-yl)phenyl]-2- |
| | nitrobenzenesulfonamide |
| N | N-[4-(2,4-dioxo-1,2,3,4,8,9,10,11-octahydro-naphtho[1,2-b][1,4]diazepin-5-yl)phenyl]- |
| | 2-nitro-benzenesulfonamide |
| O | N-[4-(2,4-dioxo-1,2,3,4,8,9,10,11-octahydro-naphtho[1,2-b][1,4]diazepin-5-yl)phenyl]- |
| | N-methyl-2-nitrobenzenesulfonamide |
| P | N-[3-(2,4-dioxo-1,2,3,4-tetrahydronaphtho[1,2-b][1,4]diazepin-5-yl)phenyl]-N-methyl- |
| | 2-nitrobenzenesulfonamide |
| Q | 4-(2,4-dioxo-1,2,3,4,8,9,10,11-octahydronaphtho[1,2-b][1,4]diazepin-5-yl)-N- |
| | phenylbenzenesulfonamide |
| R | N-[3-(2,4-dioxo-1,2,3,4-tetrahydronaphtho[1,2-b][1,4]diazepin-5-yl)phenyl]-2- |
| | naphthalenesulfonamide |
| S | N-[3-(2,4-dioxo-1,2,3,4-tetrahydronaphtho[1,2-b][1,4]diazepin-5-yl)phenyl]-1- |
| | naphthalenesulfonamide |
| T | N-[4-(2,4-dioxo-1,2,3,4-tetrahydronaphtho[1,2-b][1,4]diazepin-5- |
| | yl)phenyl]cydohexanesulfonamide |
| U | N-[4-(2,4-dioxo-1,2,3,4-tetrahydronaphtho[1,2-b][1,4]diazepin-5-yl)phenyl]-3- |
| | pyridinesulfonamide hydrochloride |
| V | N-[4-(2,4-dioxo-1,2,3,4-tetrahydronaphtho[1,2-b][1,4]diazepin-5-yl)phenyl]-4- |
| | isopropylbenzenesulfonamide |
| W | N-[4-(2,4-dioxo-1,2,3,4-tetrahydronaphtho[1,2-b][1,4]diazepin-5- |
| | yl)phenyl]phenylmethanesulfonamide |
| X | N-[4-(2,4-dioxo-1,2,3,4-tetrahydronaphtho[1,2-b][1,4]diazepin-5-yl)phenyl]-3- |
| | pyridinesulfonamide |
| Y | N-[4-(2,4-dioxo-1,2,3,4-tetrahydronaphtho[1,2-b][1,4]diazepin-5-yl)phenyl]-2- |
| | naphthalenesulfonamide |
| Z | 4-(2,4-dioxo-1,2,3,4,8,9,10,11-octahydronaphtho-[1,2-b][1,4]diazepin-5-yl)phenyl 3- |
| | bromobenzene-sulfonate |
| AA | N-benzyl-N-[4-(1-benzyl-2,4-dioxo-1,2,3,4-tetrahydronaphtho[1,2-b][1,4]diazepin-5- |
| | yl)phenyl]-2-nitrobenzenesulfonamide |
| BB | N-benzyl-N-[4-(2,4-dioxo-1,2,3,4-tetrahydronaphtho[1,2-b][1,4]diazepin-5-yl)phenyl]- |
| | 2-nitrobenzenesulfonamide |
| CC | 3-bromo-N-[4-(2,4-dioxo-1,2,3,4-tetrahydronaphtho[1,2-b][1,4]diazepin-5-yl)phenyl]- |
| | N-methylbenzenesulfonamide |
| DD | N-[4-(2,4-dioxo-1,2,3,4-tetrahydronaphtho[1,2-b][1,4]diazepin-5-yl)phenyl]-N-methyl- |
| | 2-nitrobenzenesulfonamide |
| EE | N-[4-(2,4-dioxo-1,2,3,4-tetrahydronaphtho[1,2-b][1,4]diazepin-5-yl)phenyl]-N-(2- |
| | hydroxyethyl)-2-nitrobenzenesulfonamide |
| FF | N-[4-(7-chloro-2,4-dioxo-2,3,4,5-tetrahydro-1H-benzo[b][1,4]diazepin-1- |
| | yl)phenyl]benzenesulfonamide |
| GG | N-[4-(7-bromo-2,4-dioxo-2,3,4,5-tetrahydro-1H-benzo[b][1,4]diazepin-1- |
| | yl)phenyl]benzenesulfonamide |
| HH | N-[4-[(2,4-dioxo-7-(trifluoromethyl)-2,3,4,5-tetrahydro-1H-benzo[b][1,4]diazepin-1- |
| | yl)]phenyl]benzenesulfonamide |
| II | N-[4-(2,4-dioxo-2,3,4,5-tetrahydro-1H-benzo[b][1,4]diazepin-1- |
| | yl)phenyl]benzenesulfonamide |
| JJ | 1-(2-chlorophenyl)-N-[4-(2,4-dioxo-1,2,3,4-tetrahydronaphtho[1,2-b][1,4]diazepin-5- |
| | yl)phenyl]methanesulfonamide |
| KK | 1-(3-bromophenyl)-N-[4-(2,4-dioxo-1,2,3,4-tetrahydronaphtho[1,2-b][1,4]diazepin-5- |
| | yl)phenyl]methanesulfonamide |
| LL | N-[4-(2,4-dioxo-1,2,3,4-tetrahydronaphtho[1,2-b][1,4]diazepin-5-yl)phenyl]-2- |
| | trifluoromethylbenzenesulfonamide |
| MM | N-[4-(7-b romo-6-methyl-2,4-dioxo-2,3,4,5-tetrahy dro-1H-benzo[b][1,4]diazepin-1- |
| | yl)phenyl]benzenesulfonamide |
| NN | 1-(2-chlorophenyl)-N-[4-(2,4-dioxo-1,2,3,4,8,9,10,11-octahydronaphtho[1,2- |
| | b][1,4]diazepin-5-yl)phenyl]methanesulfonamide |
| OO | 3-bromo-N-[4-(2,4-dioxo-1,2,3,4,8,9,10,11-octahydronaphtho[1,2-b][1,4]diazepin-5- |
| | yl)phenyl]benzenesulfonamide |
| PP | N-[4-(2,4-dioxo-1,2,3,4,8,9,10,11-octahydronaphtho[1,2-b][1,4]diazepin-5-yl)phenyl]-3- |
| | methoxybenzenesulfonamide |
| QQ | 1-(2-bromophenyl)-N-[4-(2,4-dioxo-1,2,3,4-tetrahydronaphtho[1,2-b][1,4]diazepin-5- |
| | yl)phenyl]methanesulfonamide |
| RR | N-[4-(2,4-dioxo-1,2,3,4-tetrahydronaphtho[1,2-b][1,4]diazepin-5-yl)phenyl]-1-(2- |
| | methylphenyl)methanesulfonamide |
| SS | N-[4-(2,4-dioxo-1,2,3,4-tetrahydronaphtho[1,2-b][1,4]diazepin-5-yl)phenyl]-1-(2- |
| | nitrophenyl)methane sulfonamide |
| TT | N-[4-(2,4-dioxo-1,2,3,4-tetrahydronaphtho[1,2-b][1,4]diazepin-5-yl)phenyl]-2- |
| | phenylethane sulfonamide |
| UU | 1-(2,3-dichlorophenyl)-N-[4-(2,4-dioxo-1,2,3,4,8,9,10,11-octahydronaphtho[1,2- |
| | b][1,4]diazepin-5-yl)phenyl]methanesulfonamide |
| VV | 1-(2-chlorophenyl)-N-[4-(2,4-dioxo-7-methoxy-1H-benzo[1,2-b][1,4]diazepin-1- |
| | yl)phenyl]methanesulfonamide |
| WW | 1-(2-chlorophenyl)-N-[4-(2,4-dioxo-7-hydroxy-1H-benzo [1,2-b][1,4]diazepin-1- |
| | yl)phenyl]methanesulfonamide |
| XX | 1-(4-chlorophenyl)-N-[4-(2,4-dioxo-1,2,3,4-tetrahydronaphtho[1,2-b][1,4]diazepin-5- |
| | yl)phenyl]methanesulfonamide |
| YY | 1-(2-chlorophenyl)-N-[4-(2,4-dioxo-1,2,3,4-tetrahydronaphtho[1,2-b][1,4]diazepin-5- |
| | yl)benzyl]methane sulfonamide |
| XX | 1-(2-chlorophenyl)-N-[4-(2,4-dioxo-1,2,3,4-tetrahydronaphtho[1,2-b][1,4]diazepin-5- |
| | yl)-2-methoxyphenyl]methanesulfonamide |
| AAA | 1-(2-chlorophenyl)-N-[4-(2,4-dioxo-1,2,3,4-tetrahydronaphtho[1,2-b][1,4]diazepin-5- |
| | yl)-2-hydroxyphenyl]methanesulfonamide |
| BBB | 1-(2,6-dichlorophenyl)-N-[4-(2,4-dioxo-1,2,3,4,8,9,10,11-octahydronaphtho[1,2- |
| | b][1,4]diazepin-5-yl)phenyl]methanesulfonamide |
| CCC | 1-(2-chlorophenyl)-N-[4-(2,4-dioxo-6-methyl-1H-benzo [1,2-b][1,4]diazepin-1- |
| | yl)phenyl]methanesulfonamide |
| DDD | N-[4-(2,4-dioxo-1,2,3,4,8,9,10,11-octahydronaphtho[1,2-b][1,4]diazepin-5-yl)phenyl]-1- |
| | (2-iodophenyl)methanesulfonamide |
| EEE | 1-(2-chlorophenyl)-N-[4-(2,4-dioxo-1,2,3,4,8,9,10,11-octahydronaphtho[1,2- |
| | b][1,4]diazepin-5-yl)phenyl]-N-methylmethanesulfonamide |
| FFF | 1-[(2-trifluoromethyl)phenyl]-N-[4-(2,4-dioxo-1,2,3,4-tetrahydronaphtho[1,2- |
| | b][1,4]diazepin-5-yl)phenyl]phenyl-N-methylmethanesulfonamide |
| GGG | 1-(2-ethylphenyl)-N-[4-(2,4-dioxo-1,2,3,4-tetrahydronaphtho[1,2-b][1,4]diazepin-5- |
| | yl)phenyl]phenyl-N-methylmethanesulfonamide |
| HHH | 1-(2,3-dimethylphenyl)-N-[4-(2,4-dioxo-1,2,3,4-tetrahydronaphtho[1,2-b][1,4]diazepin- |
| | 5-yl)phenyl]phenyl-N-methylmethanesulfonamide |
| III | 2-(2-chlorophenyl)-N-[4-(2,4-dioxo-1,2,3,4-tetrahydronaphtho[1,2-b][1,4]diazepin-5- |
| | yl)phenyl]phenyl-N-methylethanesulfonamide |
| JJJ | 1-(2-nitrophenyl)-N-[4-(2,4-dioxo-1,2,3,4-tetrahydronaphtho[1,2-b][1,4]diazepin-5- |
| | yl)phenyl]phenyl-N-methylmethanesulfonamide |
| KKK | 1-(2-aminophenyl)-N-[4-(2,4-dioxo-1,2,3,4-tetrahydronaphtho[1,2-b][1,4]diazepin-5- |
| | yl)phenyl]phenyl-N-methylmethanesulfonamide |
| LLL | 1-(2-dimethylaminophenyl)-N-[4-(2,4-dioxo-1,2,3,4-tetrahydronaphtho[1,2- |
| | b][1,4]diazepin-5-yl)phenyl]phenyl-N-methylmethanesulfonamide. |

A specific preferred compound of Formula (5) is a compound of Formula (5a).

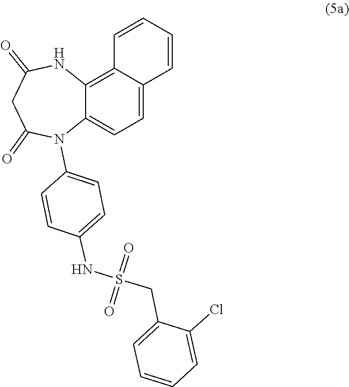 [SMILES: O=C1CC(=O)N(c2ccc(NS(=O)(=O)Cc3ccccc3Cl)cc2)c2ccc3ccccc3c2N1]

Further with respect to the above compounds of Formula (I) and Formula (5), myocardial ischemia reperfusion injury contributes to adverse cardiovascular outcomes after myocardial ischemia, cardiac surgery, or circulatory arrest. A lack of blood flow to the heart causes an imbalance between oxygen demand and supply, resulting in damage or dysfunction of the cardiac tissue. Unfortunately, restoring blood flow to the ischemic myocardium, also known as reperfusion, can also induce injury. Reperfusion following ischemia results in influx of circulating immune cells such as neutrophils and monocytes to the injured myocardium. A method of treatment by blocking the pro-inflammatory immune cell P2X4R during cardiac ischemia/reperfusion can be beneficial, and can result in reduced infarct size or restoration of cardiac performance toward normal.

Accordingly, in an aspect, a compound of Formula (I) and/or of Formula (5) is administered as a method of treatment of a human subject who has had a myocardial ischemia reperfusion injury. In a further aspect, the myocardial ischemia reperfusion injury is a result of myocardial ischemia or infarction, cardiac surgery, or circulatory arrest.

In one aspect, treatment is for a cardiac ischemia reperfusion injury from a ST elevation myocardial infarction (STEMI). In one aspect, treatment is for cardiac ischemia reperfusion injury from a non-ST elevation myocardial infarction (NSTEMI). In yet another aspect, treatment is for cardiac ischemia reperfusion injury from an acute myocardial infarction. In another aspect, cardiac dysfunction resulting from myocardial ischemia reperfusion injury is treated.

In a preferred aspect, a compound of the following formulas or a pharmaceutically acceptable salt and/or formulation thereof is administered as a method of treatment of a subject who has had a myocardial ischemia reperfusion injury:

| Compound | Structure | Salt form |
|---|---|---|
| | 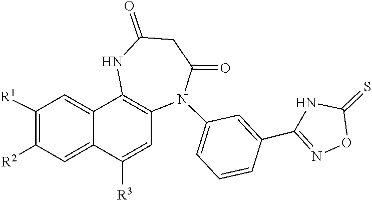 | |
| 22b | R¹ = CH₃, R² = R³ = H | Sodium salt |
| 22c^{d} | R¹ = H, R² = CH3, R³ = H | Sodium salt |
| 21d^{d} | R¹ = R² = H, R³ = Br | Triethylamine salt |
| 21e | R¹ = CH₃, R² = H, R³ = Br | Triethylamine salt |
| 21j | R¹ = H, R² = CH₂N₃, R³ = H | Triethylamine salt |
| 211 | R¹ = R² = H, R³ = Ph | Triethylamine salt |
| 21p | R¹ = R² = H, R³ = Et | Triethylamine salt |
| | 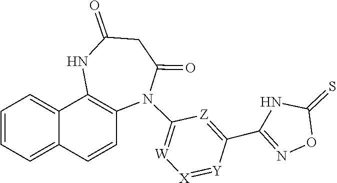 | |
| 21r | X = CCH₃, W = Y = Z = CH | Triethylamine salt |
| 21s | W = N, X = Y = Z = CH | Triethylamine salt |
| 21t | X = N, W = Y = Z = CH | Triethylamine salt |
| 21u^{d} | Y = N, W = X = Z = CH | Triethylamine salt |
| 21v | Z = N, W = X = Y = CH | Triethylamine salt |
| 5a | | Triethylamine, hydrochloride, or alkali metal such as sodium, lithium, or potassium |

In a preferred aspect, a treatment of a human subject for myocardial ischemia reperfusion injury, the compound or its pharmaceutically acceptable salt administered is of Formular 5a or of Formula Ic.

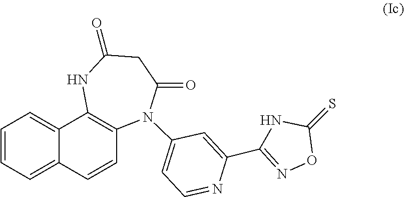 [SMILES: O=C1CC(=O)N(c2ccnc(-c3noc(=S)[nH]3)c2)c2ccc3ccccc3c2N1]

The compounds of Formula (I) and/or of Formula (5) can have one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, it should be understood that all of the optical isomers and mixtures thereof are encompassed. In addition, compounds with double bonds can occur in Z- and E-forms, with all isomeric forms of the compounds being included in the present disclosure. In these situations, the single enantiomers, i.e., optically active forms, can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column.

In an aspect, the various aspects of the above-described compounds of Formula (I) and/or of Formula (5), in particular Formulas (Ia), (Ib), or (Ic) and Formula (5a), are an antagonist of the P2X4 receptor, particularly hP2X4R. The compounds of Formula (I) and/or of Formula (5), in particular Formulas (Ia), (Ib), or (Ic) and Formula (5a), can have an IC₅₀ of 10 to 5,000 nM, or 50 to 2,500 nN, or 50 to 1,000 nM. In an aspect, certain of the compounds Formula (I) and/or Formula (5), in particular Formulas (Ia), (Ib), or (Ic) and Formula (5a), can have an IC₅₀ of 10 to 5,000 nM, or 50 to 2,500 nM, or 50 to 1,000 nM, and a negligible effect as an antagonist of P2X receptors other than P2X4, particularly other than hP2X4R.

Specific methods for synthesis of compounds of Formula (5) are known to those of ordinary skill in the art, being described, for example, in U.S. Pat. No. 11,434,207 at columns 16-28. Specific methods for synthesis of the compounds of Formula (I) are described in the Examples herein. The syntheses of compounds of Formula (I), in particular compounds 22a-c, were performed largely by following the route reported by Sakuma et al. as described in US2011319610A1 (2011)). However, to accomplish the envisaged SAR, it was necessary to adapt other synthetic protocols in the schemes. The general synthetic route is shown in Scheme 1.

For nitration of 2-naphthols (7a-c, Scheme 1), traditional nitration using nitric acid with acetic acid or dichloromethane as solvent at room temperature (rt) resulted in good isolated yield (61%). Nitronaphthols 8a-c (Scheme 1) were then sulfonylated, and the products was subjected to Buchwald-Hartwig amination to give 10a-c. The nitro groups in these compounds were reduced to amines by catalytic hydrogenation to give 1,2-diaminonaphthalene derivatives 11a-c. Compound 11a was subjected to bromination, which gave 4-bromonaphthalene derivative 11d, and the structure was confirmed by 1D and 2D NMR experiments. The 1,2-diamino compounds were converted to the corresponding diazepinodiones 13a-d in moderate yields by reacting with malonyl chloride in refluxing toluene. Compounds 11d and 13d were prepared via bromination of 11a and 13a at slightly elevated temperatures in comparable yields. To increase the formation of 13a, a sequential addition of monomethyl malonate, DCC, potassium t-butoxide gave a cyclized moiety in a slightly improved conversion (51%) compared to a malonyl chloride procedure (40%) (Jagtap et al., WO2016151464A1, 2016). A similar one-pot procedure using ethyl chloromalonate gave only low product amounts. However, a two-step protocol improved the yield to 70% overall, in which ethyl chloromalonate was coupled to the α-amino group, and the intermediate purified and reacted with potassium t-butoxide to form diazepine analogue 13a.

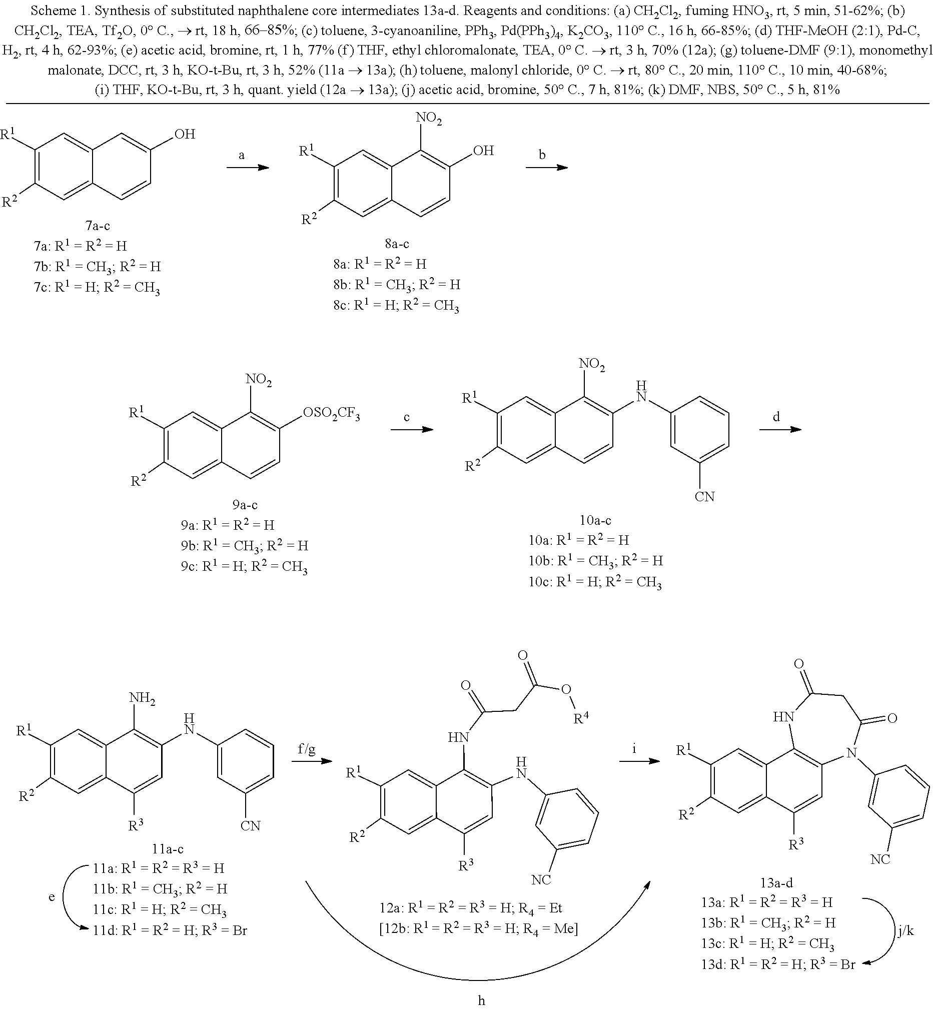

To add functionality to the naphthalene core, the position 6/7-methyl groups in 13b and 13c were modified as shown in Scheme 2. Among various benzylic bromination methods, NBS, (BzO)₂, CH₂Cl₂/benzene gave the best conversion and isolated yields ranging from 40-65%. However, this process also gave varying amounts of 4-bromo derivatives (13e, 14b and 16b), which were inseparable using normal silica gel chromatography. The benzyl bromides were reacted with nucleophiles to give the methylthio analogue 13f, and azide analogues 13g, h, j, and k. The azide group in 7-azidomethylene analogue 13g was converted to an amine by the Staudinger reduction, and the amine was acetylated to give 13i.

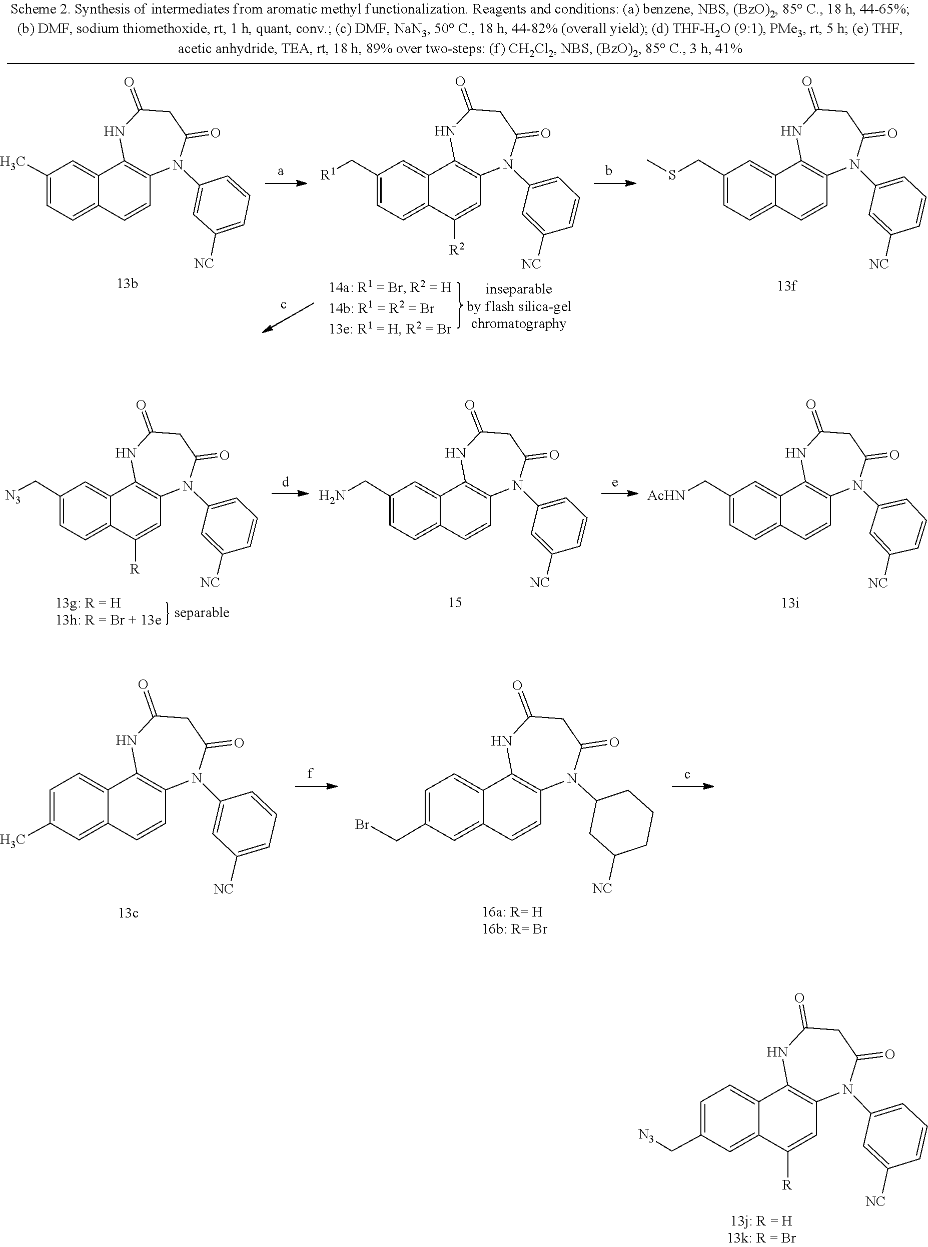

The bromine at the 4-position of naphthalene was used to add aryl, alkane, alkyne, and arylalkyne/heteroarylalkyne groups as shown in Scheme 3. Palladium-catalyzed Suzuki coupling gave 13l, while a Sonogashira coupling gave 13m-o and 17. A direct Pd—C hydrogenation of 17 gave trimethylsilylethylene derivative 18, while TBAF-mediated deprotection of TMS followed by catalytic saturation gave 4-ethylnaphthalene derivative 13p.

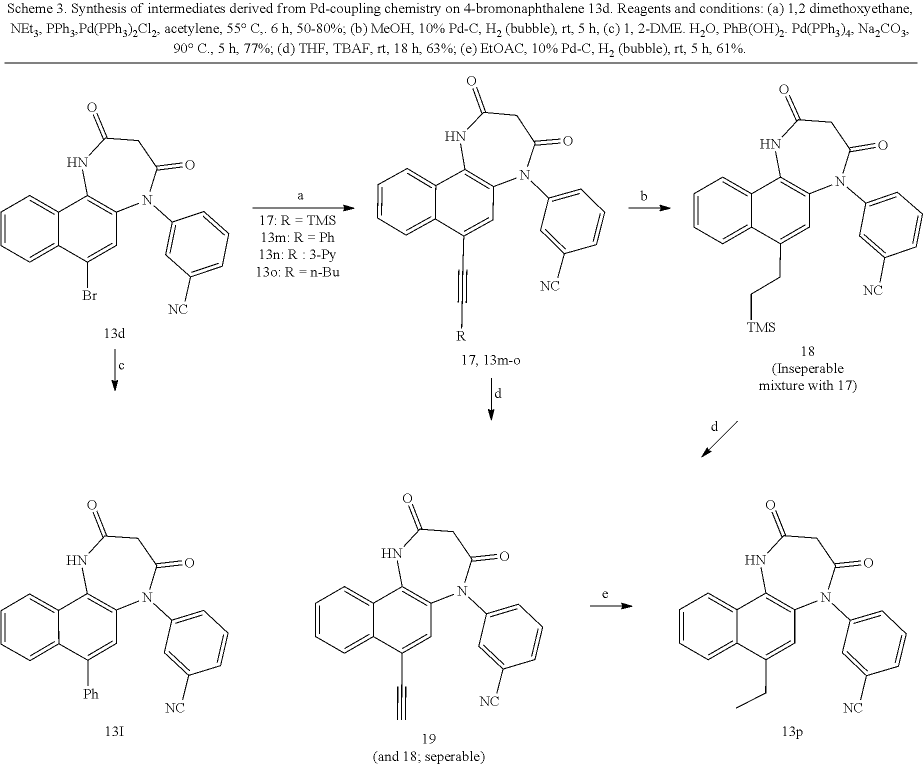

The nitrile groups in compounds 13a-p were converted to N-hydroxyimidamides by reaction with hydroxylamine hydrochloride in the presence of triethylamine as base (Scheme 4). Reacting these compounds with thiocarbonydiimidazole (thio-CDI) in presence of DBU gave compounds 21a-p. The amino-analogue 21q was made from the corresponding azido-analogue 21g by trimethylphosphine mediated Staudinger reduction. For better solubility, the compounds can be converted to it sodium salt (22a-c) by reacting with equimolar amount of dilute sodium hydroxide followed by lyophilization of the solvents.

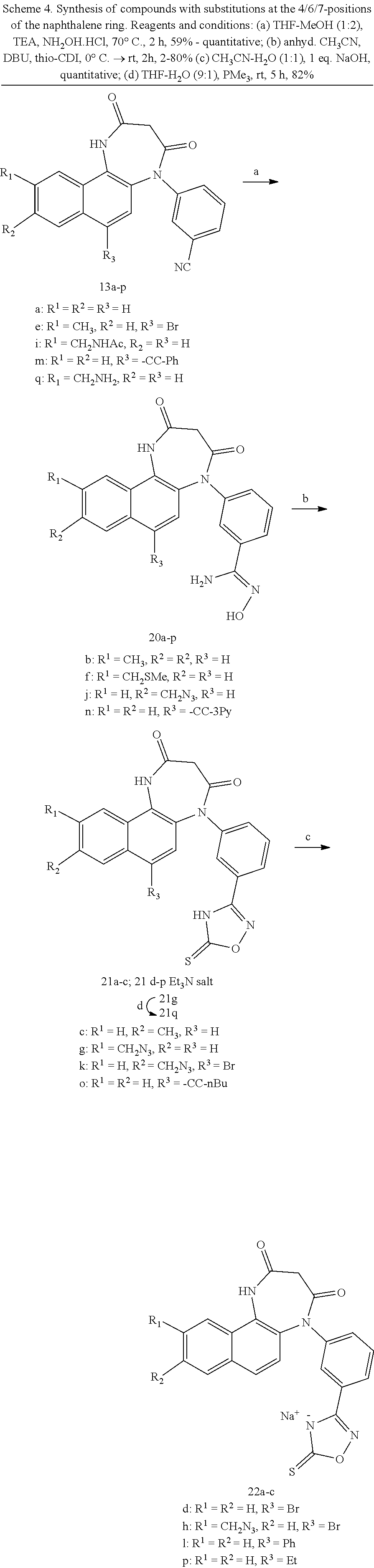

Synthesis of toluidine analogue 21r proceeded with ease (Scheme 5). However, synthesi13 ns of the aza-analogues was more difficult. Initial efforts to couple isonicotinonitrile with 13 nitronaphthol triflate 9a failed, which led to screening of various higher generation Pd-catalysts and ligands, bases, and solvents for the Buchwald-Hartwig reaction. The use of 1,4-dioxane as solvent dramatically increased yield. The method of choice for synthesis of 10s-v was 1,4-dioxane, hetAr-NH₂ (1.5 eq), Pd(PPh₃)₄ (0.1 eq), PPh₃ (1.0 eq), Cs₂CO₃ (1.5 eq), at 110° C., for 18 hours, although it gave a relatively slightly lower yield due to more challenging purification. After nitro reduction to an amine, malonyl chloride in various solvent systems were tried. Pyridine as solvent degraded the starting material, while 3:1 toluene-dioxane proceeded with slightly higher yield compared to 2:1 toluene-1,2-DCE. It was found that 13s treated with 2 eq. hydroxylamine at room temperature for 2 h yielded the desired 20s as the major product. This hydroxyimidamide was reacted with thio-CDI to produce 21s in moderate yields. The similar reaction sequence to generate 21s from 9a lead to the synthesis of other aza-analogues 21t-v in acceptable yields.

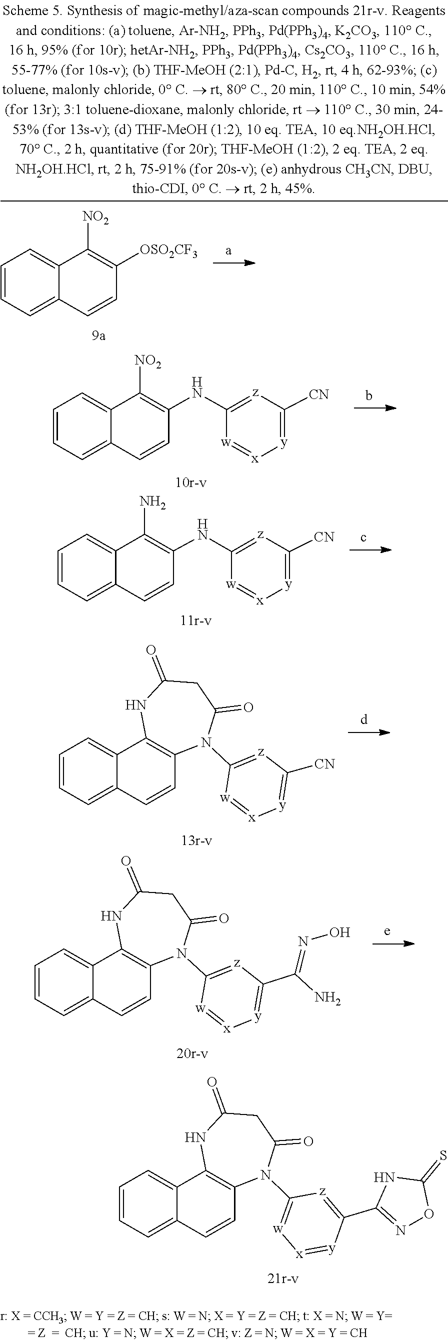

Twenty compounds were tested for inhibition of human (h) P2XRs (Table 3), including at homotrimeric P2X1R, P2X3R, and P2X4R and at heteromeric P2X2/3R. The assays used HEK-293 cells stably transfected with P2RX4R and CHO-K1 cells stably transfected with either P2X1R, P2X2/3R, and P2X3R in 96-well and 386-well plates. Detection of each well was by luminescence or fluorescence using an imaging plate reader. Full dose response curves were determined for both reference agonist and antagonist at each receptor. The IC50 values reported for the test compounds represent the inhibition of a ˜EC80 concentration of the reference agonist. A robust Z′ (RZ′) parameter was determined for each assay plate and ranged from 0.683 to 0.903, which indicated the robust quality of the assay. The antagonists were not evaluated at the mouse (m) P2X4R, but we note that the lead compound 5 was as potent in blocking mP2X4R effects in vitro as at hP2X4R and also showed considerable in vivo efficacy in the mouse.

In an aspect, a method for treatment of a human subject who has had a stroke comprises administering to the subject a pharmaceutical composition comprising a compound of Formula (I) and/or Formula (5), in particular Formulas (Ia), (5a), (Ib), or (Ic). In an aspect, the stroke is an ischemic stroke. In another aspect, the stroke is a hemorrhagic stroke or a transient ischemic attack (TIA).

The compounds of Formula (I) and/or Formula (5), in particular Formulas (Ia), (5a), (Ib), or (Ic), or their pharmaceutically acceptable salts and/or formulations thereof can be formulated with an adjuvant to provide the pharmaceutical composition. Suitable adjuvants depend on the delivery method and form, and are described in more detail below.

The pharmaceutical composition comprising a compound of Formula (I) and/or Formula (5), in particular Formulas (Ia), (5a), (Ib), or (Ic), or a pharmaceutically acceptable salt and/or formulation thereof, can be administered during the acute phase of stroke, between the time the stroke occurs and lasting for up to about 7 days after stroke. In an aspect, administration of the pharmaceutical composition comprising a compound Formula (I) and/or Formula (5), in particular Formulas Ia, 5a, Ib, or Ic, or a pharmaceutically acceptable salt and/or formulation thereof, is ceased after the acute phase of stroke, i.e., after 7 days post-stroke. In another aspect, the pharmaceutical composition comprising a compound Formula (I) and/or Formula (5), in particular Formulas (Ia), (5a), (Ib), or (Ic), or a pharmaceutically acceptable salt and/or formulation thereof, is administered in the acute phase, but ceased 1 day post-stroke, 2 days post-stroke, 3 days post-stroke, 4 days post-stroke, 5 days post-stroke, 6 days post-stroke, or 7 days post-stroke. In another aspect, the pharmaceutical composition comprising a compound Formula (I) and/or Formula (5), in particular Formulas (Ia), (5a), (Ib), or (Ic), or a pharmaceutically acceptable salt and/or formulation thereof, is administered in the acute, the subacute, the chronic phase of stroke, or a combination comprising thereof. Administration during the chronic phase of stroke is expected to be beneficial.

In an aspect, administering a pharmaceutical composition comprising a compound of Formula (I) and/or Formula (5), in particular Formulas Ia, (5a), Ib, or Ic, or a pharmaceutically acceptable salt and/or formulation thereof, can be by oral administration, for example, administration of a solid or liquid oral pharmaceutical formulation.

In another aspect, administering a pharmaceutical composition comprising a compound of Formula (I) and/or Formula (5), in particular Formulas Ia, 5a, Ib, or Ic, or a pharmaceutically acceptable salt and/or formulation thereof, can be by intravenous injection, such as injection into the general circulation or by targeted infusion whereby the agent is slowly supplied close to the site of the blockage that triggered the stroke. Infusion can be via an endovascular catheter such as a catheter ready to be used, being used, or having been used in providing a thrombolytic therapeutic to the subject; or a catheter having been used in conjunction with a procedure on the subject involving use of a clot-removal device.

A pharmaceutical composition comprising a compound of Formula (I) and/or Formula (5), in particular Formulas (Ia), (5a), (Ib), or (Ic), or a pharmaceutically acceptable salt and/or formulation thereof, can be administered one minute to up to 3 hours before administering a thrombolytic therapeutic or clot retrieval mechanically via an endovascular approach (also known as mechanical lysis) to the subject. A pharmaceutical composition comprising a compound of Formula (I) and/or Formula (5), in particular Formulas (Ia), (5a), (Ib), or (Ic), or a pharmaceutically acceptable salt and/or formulation thereof, can be administered concomitantly with a thrombolytic therapeutic or clot retrieval mechanically via an endovascular approach to the subject. Alternatively, a pharmaceutical composition comprising a compound of Formula (I) and/or Formula (5), in particular Formulas (Ia), (5a), (Ib), or (Ic), or a pharmaceutically acceptable salt and/or formulation thereof, can be administered after a thrombolytic therapeutic or clot retrieval mechanically via an endovascular approach to the subject. Thrombolytic therapeutics include compounds such as aspirin, clopidogrel, triclopidine, tissue plasminogen activator, urokinase, and streptokinase. A combination thereof can be used.

For oral administration, the pharmaceutical composition can be in liquid form, for example, solutions, syrups, or suspensions, or can be presented as a drug product for reconstitution with water or other suitable vehicle before use. Such liquid preparations can be prepared by conventional means with pharmaceutically acceptable additives (adjuvants) such as suspending agents (e.g., sorbitol syrup, cellulose derivatives, or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The pharmaceutical compositions can take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well-known in the art.

Pharmaceutical compositions for oral administration can be suitably formulated to give controlled release of the active compound.

For buccal administration, the pharmaceutical compositions can take the form of tablets or lozenges formulated in conventional manner.

For administration by inhalation, the pharmaceutical compositions can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The pharmaceutical compositions can be formulated for parenteral administration by injection, e.g., by bolus injection or continuous infusion via either intravenous, intraperitoneal, or subcutaneous injection. Many of the injectable formulations have their own specific co-solvents or excipients, which may or may not be in addition to the salts that conjugate with the drug substance. Pharmaceutical compositions for injection can be presented in unit dosage form, e.g., in ampoules or in multi-dose containers, with an added preservative. The compositions can take such forms as suspensions, solutions, or emulsions in oily or aqueous vehicles, and can contain formulatory agents such as suspending, stabilizing, or dispersing agents, or a combination thereof. Alternatively, the compound of Formula (I) and/or Formula (5), in particular Formulas (Ia,) (Ib), (5a), or (Ic), or a pharmaceutically acceptable salt thereof and/or formulation, can be in powder form for constitution with a suitable vehicle, e.g., sterile pyrogen-free water, before use.

In addition to the compositions described above, the pharmaceutical compositions can also be formulated as a depot preparation. Such long-acting formulations can be administered by implantation (e.g., subcutaneously or intramuscularly) or by intramuscular injection. Thus, for example, the pharmaceutical compositions can be formulated with suitable polymeric or hydrophobic materials (e.g., as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

The pharmaceutical compositions can, if desired, be presented in a pack or dispenser device, which can contain one or more unit dosage forms containing the active ingredient. The pack can for example comprise metal or plastic foil, such as a blister pack. The pack or dispenser device can be accompanied by instructions for administration.

The amount of a compound of Formula (I) and/or Formula (5), in particular Formulas (Ia), (Ib), (Ic), or (5c), or a pharmaceutically acceptable salt and/or formulation thereof that can be combined with pharmaceutically acceptable adjuvant to produce a single dosage form can vary depending upon the host treated and the particular mode of administration. The specific therapeutically effective amount for a particular patient will depend on a variety of factors, including the activity of the specific compound employed, the age, body weight, general health, sex, diet, time of administration, route of administration, rate of excretion, drug combination, and the severity of the particular disease undergoing therapy. In some instances, dosage levels below the lower limit of the aforesaid range can be more than adequate, while in other cases still larger doses can be used without causing any harmful side effects, provided that such higher dose levels are first divided into several small doses for administration throughout the day. The concentrations of the compounds described herein found in therapeutic compositions will vary depending upon a number of factors, including the dosage of the drug to be administered, the chemical characteristics (e.g., hydrophobicity) of the compounds employed, and the route of administration. In an aspect, a pharmaceutical composition including a compound of Formula (I) and/or Formula (5), in particular Formulas (Ia), (Ib), (5c), or (Ic), or a pharmaceutical salt thereof, is administered at a dosage of about 0.05 mg/kg to about 0.5 mg/kg to about 5 mg/kg of body weight of the subject.

The invention is further illustrated by the following non-limiting examples.

### Examples

### Chemical Synthesis

Reagents and instrumentation: 6/7-Methyl-naphthalene-2-ol derivatives and aminopyridinenitriles were purchased from Enamine (New Jersey, USA). All other chemicals and solvents were from Sigma-Aldrich (St. Louis, MO, USA). Anhydrous solvents were obtained directly from commercial sources. All reactions were carried out under argon using anhydrous solvents. Room temperature or rt refers to 25±2° C. NMR spectra were recorded on a Bruker 400 MHz spectrometer. Chemical shifts are given in ppm (δ), calibrated to the residual solvent or TMS signals for hydrogen, carbon, and internally calibrated by solvent frequency for other nuclei (MestReNova 10.0.2). Exact mass measurements were performed on a proteomics optimized Q-TOF-2 (Micromass-Waters) mass spectrometer equipped with a standard electrospray ionization (ESI) and modular LockSpray™ interface. The RP-HPLC was performed using a Phenomenex Luna 5 μm C18(2)100A, AXIA, 21.2×250 mm column. Purity was determined using Agilent C18-XDB, 5 μm, 4.6×250 mm column, and a 0 to 100% linear gradient of acetonitrile/10 mM triethylammonium acetate as mobile phase at 1 mL/min flow rate for 20 min. Purity of all the tested compounds were >95% at 254 nm and/or respective absorption wavelength in nm, unless noted otherwise.

1-Nitronaphthalen-2-ol (8a): To a solution of 2-naphthol (100 mg, 0.69 mmol) in dichloromethane (2 mL) at room temperature was added fuming nitric acid (>99.5% HNO₃, 32 μL, 0.08 mmol) and the mixture stirred for additional 5 min. Silica gel (0.5 g) was added and volatiles evaporated to adsorb on it. Purification by silica gel chromatography afforded 8a as yellow crystals (80 mg, 61%, R_{f}=0.3, TLC eluent=10% ethyl acetate in hexanes). ¹H NMR (400 MHZ, CDCl₃) δ 12.19 (s, 1H), 8.93 (d, J=8.8 Hz, 1H), 8.01 (d, J=9.0 Hz, 1H), 7.83 (dd, J=8.1, 1.3 Hz, 1H), 7.74 (ddd, J=8.6, 7.0, 1.4 Hz, 1H), 7.51 (ddd, J=8.0, 6.9, 1.0 Hz, 1H), 7.27 (d, J=9.0 Hz, 1H). HRMS m/z [M−H]⁻ for C₁₀H₇O₃N calculated 188.0348, found 188.0346.

7-Methyl-1-nitronaphthalen-2-ol (8b): Following the procedure described for the synthesis of 8a, compound 7b (100 mg, 0.63 mmol) gave 8b as yellow crystals (65 mg, 51%, R_{f}=0.3, TLC eluent=10% ethyl acetate in hexanes). ¹H NMR (400 MHZ, CDCl₃) δ 12.21 (s, 1H), 8.74 (s, 1H), 7.95 (d, J=9.0 Hz, 1H), 7.71 (d, J=8.1 Hz, 1H), 7.34 (dd, J=8.3, 1.5 Hz, 1H), 7.18 (d, J=9.0 Hz, 1H), 2.59 (s, 3H). HRMS m/z [M−H]⁻ for C₁₁H₉O₃N calculated 202.0504, found 202.0504.

6-Methyl-1-nitronaphthalen-2-ol (8c): Following the procedure described for the synthesis of 8a, compound 7c (150 mg, 0.95 mmol) gave 8c as yellow crystals (101 mg, 52%, R_{f}=0.3, TLC eluent=10% ethyl acetate in hexanes). ¹H NMR (400 MHZ, CDCl₃) δ 12.21 (s, 1H), 8.83 (d, J=8.9 Hz, 1H), 7.93 (d, J=9.1 Hz, 1H), 7.60 (s, 1H), 7.57 (d, J=9.2 Hz, 1H), 7.23 (d, J=9.0 Hz, 1H), 2.51 (s, 3H). HRMS m/z [M−H]⁻ for C₁₁H₉O₃N calculated 202.0504, found 202.0507.

1-Nitronaphthalen-2-yl trifluoromethanesulfonate (9a): To a solution of 8a (190 mg, 1.0 mmol) in dichloromethane (5 mL) at 0° C. was added triethylamine (0.35 mL, 2.5 mmol), followed by trifluoromethanesulfonic anhydride (0.2 mL, 1.2 mmol) and the mixture stirred at room temperature for 2 h. The reaction mixture was partitioned between water-dichloromethane. The organic layer was separated, dried over anhydrous Na₂SO₄, and evaporated. The residue was purified by silica gel chromatography to afford 9a as a light-yellow solid (275 mg, 85%, R_{f}=0.25, TLC eluent=10% ethyl acetate in hexanes; Note: if the reaction is incomplete, NaHCO₃ workup would leave starting material in aqueous medium, and 9a was extracted into the organic phase, while the starting material 8a was recovered after acidification, followed by extraction). ¹H NMR (400 MHZ, CDCl₃) δ 8.12 (d, J=9.2 Hz, 1H), 7.99 (d, J=8.2 Hz, 1H), 7.95-7.89 (m, 1H), 7.77 (ddd, J=8.5, 6.9, 1.3 Hz, 1H), 7.71 (ddd, J=8.1, 7.0, 1.2 Hz, 1H), 7.55 (d, J=9.2 Hz, 1H). ¹⁹F NMR (376 MHZ, CDCl₃) δ −73.04. HRMS m/z [M−H]⁻ for C₁₁H₅NO₅F₃S calculated 319.9841, found 319.9842.

7-Methyl-1-nitronaphthalen-2-yl trifluoromethanesulfonate (9b): Following the procedure described for the synthesis of 9a, compound 8b (65 mg, 0.32 mmol) gave 9b as yellow crystals (85 mg, 80%, R_{f}=0.3, TLC eluent=10% ethyl acetate in hexanes). ¹H NMR (400 MHZ, chloroform-d) δ 8.05 (d, J=9.1 Hz, 1H), 7.87 (d, J=8.4 Hz, 1H), 7.66 (s, 1H), 7.53 (d, J=8.5 Hz, 1H), 7.46 (d, J=9.1 Hz, 1H), 2.58 (s, 3H). ¹⁹F NMR (376 MHZ, chloroform-d) δ −73.09.

6-Methyl-1-nitronaphthalen-2-yl trifluoromethanesulfonate (9c): Following the procedure described for the synthesis of 9a, compound 8c (180 mg, 0.886 mmol) gave 9c as yellow crystals (195 mg, 66%, R_{f}=0.3, TLC eluent=10% ethyl acetate in hexanes). ¹H NMR (400 MHZ, chloroform-d) δ 8.01 (d, J=9.1 Hz, 1H), 7.82 (d, J=8.8 Hz, 1H), 7.75 (s, 1H), 7.59 (d, J=8.7 Hz, 1H), 7.50 (d, J=9.1 Hz, 1H), 2.58 (s, 3H). ¹⁹F NMR (376 MHZ, chloroform-d) δ −73.09.

3-((1-Nitronaphthalen-2-yl)amino)benzonitrile (10a): To an RB Flask with a stir bar was added 9a (275 mg, 0.856 mmol), triphenylphosphine (PPh₃, 225 mg, 0.856 mmol), tetrakis(triphenylphosphine)palladium (Pd(PPh₃)₄, 99 mg, 0.086 mmol), potassium carbonate (K₂CO₃, 119 mg, 0.856 mmol), and 3-cyanoaniline (152 mg, 1.284 mmol). The flask was evacuated and filled with nitrogen, followed by toluene (10 mL), and the mixture was heated to 110° C. for 16 h. The solvent was evaporated, and the residue partitioned between dichloromethane and water. Organic layer was separated, washed with 0.2M hydrochloric acid, brine, and dried over anhydrous Na₂SO₄. The solvent was evaporated, and the residue purified by silica gel chromatography to afford 10a as brown/orange crystals (210 mg, 85%, R_{f}=0.25, TLC eluent=50% CH₂Cl₂ in hexanes). ¹H NMR (400 MHZ, chloroform-d) δ 9.00 (s, 1H), 8.38 (d, J=8.7 Hz, 1H), 7.87 (d, J=9.2 Hz, 1H), 7.78 (d, J=8.1 Hz, 1H), 7.66 (ddd, J=8.6, 7.0, 1.4 Hz, 1H), 7.57-7.43 (m, 5H), 7.37 (d, J=9.1 Hz, 1H). HRMS m/z [M+H]⁺ for C₁₇H₁₂N₃O₂ calculated 290.0930, found 290.0929.

3-((7-Methyl-1-nitronaphthalen-2-yl)amino)benzonitrile (10b): Following the procedure described for the synthesis of 10a, compound 9b (80 mg, 0.24 mmol) gave 10b as a brown-orange crystal (45 mg, 62%, R_{f}=0.25, TLC eluent=50% CH₂Cl₂ in hexanes). ¹H NMR (400 MHZ, chloroform-d) δ 8.91 (s, 1H), 8.15 (s, 1H), 7.81 (d, J=9.1 Hz, 1H), 7.67 (d, J=8.2 Hz, 1H), 7.56-7.41 (m, 4H), 7.34-7.28 (m, 2H), 2.55 (s, 3H). HRMS m/z [M+H]⁺ for C₁₈H₁₃N₃O₂ calculated 304.1086, found 304.1083.

3-((6-Methyl-1-nitronaphthalen-2-yl)amino)benzonitrile (10c): Following the procedure described for the synthesis of 10a, compound 9c (195 mg, 0.582 mmol) gave 10c as a brown-orange crystal (120 mg, 68%, R_{f}=0.25, TLC eluent=50% CH₂Cl₂ in hexanes). ¹H NMR (400 MHZ, chloroform-d) δ 8.98 (s, 1H), 8.29 (d, J=8.8 Hz, 1H), 7.79 (d, J=9.1 Hz, 1H), 7.56 (s, 1H), 7.54-7.41 (m, 4H), 7.35 (d, J=9.1 Hz, 1H), 2.50 (s, 3H).

3-Methyl-5-((1-nitronaphthalen-2-yl)amino)benzonitrile (10r): Following the procedure described for the synthesis of 10a, compound 9a (100 mg, 0.311 mmol) gave 10r as an orange crystal (90 mg, 95%, R_{f}=0.25, TLC eluent=50% CH₂Cl₂ in hexanes. ¹H NMR (400 MHz, chloroform-d) δ 9.04 (s, 1H), 8.42 (d, J=8.8 Hz, 1H), 7.87 (d, J=9.1 Hz, 1H), 7.79 (d, J=8.0 Hz, 1H), 7.67 (dd, J=8.6, 7.0 Hz, 1H), 7.47 (t, J=7.5 Hz, 1H), 7.41-7.34 (m, 2H), 7.29 (s, 2H), 2.43 (s, 3H).

2-((1-Nitronaphthalen-2-yl)amino)isonicotinonitrile (10s): Compound 9a (50 mg, 0.156 mmol), 2-amino-4-cyanopyridine (28 mg, 0.233 mmol), Pd(PPh₃)₄ (18 mg, 0.016 mmol), triphenylphosphine (41 mg, 0.156 mmol) and Cs₂CO₃ (76 mg, 0.233 mmol) where charged to round-bottomed flask, and de-oxygenated with vacuum-argon cycles. Dioxane (2 mL) was added, and the mixture stirred at 110° C. for 18 h. The solvent was evaporated, and the residue partitioned between dichloromethane and water. Organic layer was separated, washed with brine, and dried over anhydrous Na₂SO₄. The solvent was evaporated, and the residue purified by silica gel chromatography to afford 10s as brown/orange crystals (30 mg, 66%, R_{f}=0.25, TLC eluent=CH₂Cl₂). ¹H NMR (400 MHz, chloroform-d) δ 8.88 (s, 1H), 8.49-8.42 (m, 1H), 8.17 (dd, J=9.0, 6.6 Hz, 2H), 8.01 (d, J=9.1 Hz, 1H), 7.87 (d, J=8.0 Hz, 1H), 7.68 (ddd, J=8.5, 7.0, 1.4 Hz, 1H), 7.54 (ddd, J=8.1, 6.9, 1.1 Hz, 1H), 7.17 (t, J=1.1 Hz, 1H), 7.14 (dd, J=5.1, 1.3 Hz, 1H). HRMS m/z [M+H]⁺ for C₁₆H₁₀N₄O₂ calculated 291.0882, found 291.0881.

5-((1-Nitronaphthalen-2-yl)amino) nicotinonitrile (10t): Following the procedure described for the synthesis of 10s, compound 9a (100 mg, 0.311 mmol) gave 10t as an orange crystal (50 mg, 55%, R_{f}=0.60, TLC eluent=2% MeOH in CH₂Cl₂). ¹H NMR (400 MHZ, chloroform-d) δ 8.76 (d, J=2.7 Hz, 1H), 8.72 (s, 1H), 8.64 (d, J=1.8 Hz, 1H), 8.32 (d, J=8.7 Hz, 1H), 7.96 (d, J=9.1 Hz, 1H), 7.89-7.77 (m, 2H), 7.71 (ddd, J=8.5, 6.9, 1.4 Hz, 1H), 7.58-7.49 (m, 1H), 7.40 (d, J=9.1 Hz, 1H).

4-((1-Nitronaphthalen-2-yl)amino) picolinonitrile (10u): Following the procedure described for the synthesis of 10s, compound 9a (200 mg, 0.63 mmol) gave 10u as a yellow solid (140 mg, 77%, R_{f}=0.30, TLC eluent=100% CH₂Cl₂). ¹H NMR (400 MHZ, chloroform-d) δ 8.51 (d, J=5.6 Hz, 1H), 8.18-8.10 (m, 2H), 8.06 (d, J=9.0 Hz, 1H), 7.92 (d, J=8.1 Hz, 1H), 7.73 (ddd, J=8.6, 7.0, 1.4 Hz, 1H), 7.64-7.56 (m, 2H), 7.40 (d, J=2.3 Hz, 1H), 7.16 (dd, J=5.7, 2.4 Hz, 1H). HRMS m/z [M+H]⁺ for C₁₆H₁₀N₄O₂ calculated 291.0882, found 291.0883.

4-((1-Nitronaphthalen-2-yl)amino) picolinonitrile (10v): Following the procedure described for the synthesis of 10s, compound 9a (200 mg, 0.63 mmol) gave 10v as a yellow solid (140 mg, 77%, R_{f}=0.20, TLC eluent=50% CH₂Cl₂ in hexanes). ¹H NMR (400 MHZ, chloroform-d) δ 9.04 (s, 1H), 8.40 (d, J=9.2 Hz, 1H), 8.17 (d, J=8.7 Hz, 1H), 8.02 (d, J=9.2 Hz, 1H), 7.88 (d, J=8.1 Hz, 1H), 7.74 (dd, J=8.5, 7.4 Hz, 1H), 7.67 (ddd, J=8.6, 6.9, 1.3 Hz, 1H), 7.53 (ddd, J=8.1, 6.9, 1.1 Hz, 1H), 7.35 (d, J=7.4 Hz, 1H), 7.11 (d, J=8.4 Hz, 1H). HRMS m/z [M+H]⁺ for C₁₆H₁₀N₄O₂ calculated 291.0882, found 291.0880.

3-((1-Aminonaphthalen-2-yl)amino)benzonitrile (11a): To a solution of compound 10a (210 mg, 0.725 mmol) in 1:2 methanol-THF (12 mL) was added 10% Pd—C(100 mg) and a stream of hydrogen was bubbled through for 4 h at room temperature. The catalyst was filtered off, and the filtrate was concentrated under vacuum. The residue was purified by silica gel chromatography to afford 5a as yellowish solid (166 mg, 88%, R_{f}=0.50, TLC eluent=100% CH₂Cl₂). ¹H NMR (400 MHZ, chloroform-d) δ 8.33-8.12 (m, 3H), 7.98-7.80 (m, 2H), 7.72 (d, J=8.6 Hz, 1H), 7.67-7.57 (m, 3H), 7.45 (d, J=7.5 Hz, 1H), 7.27 (d, J=6.7 Hz, 1H), 5.87 (s, 2H). HRMS m/z [M+H]⁺ for C₁₇H₁₄N₃ calculated 260.1188, found 260.1184.

3-((1-Amino-7-methylnaphthalen-2-yl)amino)benzonitrile (11b): Following the procedure described for the synthesis of 11a, compound 10b (45 mg, 0.148 mmol) gave 11b as a yellowish solid (25 mg, 62%, R_{f}=0.5, TLC eluent=100% CH₂Cl₂). ¹H NMR (400 MHZ, chloroform-d) δ 8.13 (dt, J=8.4, 2.1 Hz, 1H), 8.03 (s, 1H), 7.78-7.58 (m, 3H), 7.54 (dt, J=8.5, 2.2 Hz, 1H), 7.49-7.40 (m, 1H), 7.27 (d, J=9.2 Hz, 2H), 5.86 (s, 1H), 4.73 (s, 2H), 2.97 (d, J=2.4 Hz, 3H). HRMS m/z [M]⁺ for C₁₈H₁₅N₃ calculated 273.1266, found 273.1266.

3-((1-Amino-6-methylnaphthalen-2-yl)amino)benzonitrile (11c): Following the procedure described for the synthesis of 11a, compound 10c (120 mg, 0.396 mmol) gave 11c as a yellowish solid (100 mg, 93%, R_{f}=0.5, TLC eluent=100% CH₂Cl₂). ¹H NMR (400 MHZ, chloroform-d) δ 7.74 (d, J=8.6 Hz, 1H), 7.59 (s, 1H), 7.38-7.32 (m, 1H), 7.23 (d, J=8.4 Hz, 3H), 7.17 (d, J=8.6 Hz, 1H), 7.05 (d, J=7.6 Hz, 1H), 6.87 (d, J=6.9 Hz, 2H), 5.43 (s, 1H), 4.36 (s, 2H), 2.52 (s, 3H). MS m/z [M+H]⁺ for C₁₈H₁₅N₃ calculated 274.1339, found 274.1.

3-((1-Amino-4-bromonaphthalen-2-yl)amino)benzonitrile (11d): Compound 11a (13 mg, 0.05 mmol) was dissolved in glacial acetic acid (0.3 mL) and was added bromine (3 μL, 0.06 mmol). The reaction mixture was stirred for 0.5-1 h at room temperature and solvent was evaporated under reduced pressure. The residue was partitioned between sat. NaHCO₃ and ethyl acetate. Organic layer separated, dried over anhydr. Na₂SO₄, evaporated. The residue was purified by silica gel column chromatography to afford 11d as a dark green solid (13 mg, 77%, R_{f}=0.5, TLC eluent=100% CH₂Cl₂). ¹H NMR (400 MHZ, DMSO-d₆) δ 8.21 (d, J=8.4 Hz, 1H), 8.00 (d, J=8.0 Hz, 1H), 7.92 (s, 1H), 7.59 (t, J=7.4 Hz, 1H), 7.56-7.45 (m, 2H), 7.31 (t, J=8.0 Hz, 1H), 7.07 (d, J=7.5 Hz, 1H), 6.90 (d, J=8.2 Hz, 1H), 6.85 (s, 1H). HRMS m/z [M+H]⁺ for C₁₇H₁₂BrN₃ calculated 338.0199, found 338.0202.

3-((1-Aminonaphthalen-2-yl)amino)-5-methylbenzonitrile (11r): Following the procedure described for the synthesis of 11a, compound 10r (90 mg, 0.297 mmol) gave 11r as a greenish solid (51 mg, 63%, R_{f}=0.3, TLC eluent=100% CH₂Cl₂). ¹H NMR (400 MHZ, chloroform-d) δ 7.92-7.80 (m, 2H), 7.52 (tt, J=7.4, 3.5 Hz, 2H), 7.34 (d, J=8.6 Hz, 1H), 7.22 (d, J=8.6 Hz, 1H), 6.90 (s, 1H), 6.70 (dt, J=10.0, 1.8 Hz, 2H), 5.40 (s, 1H), 4.40 (s, 2H), 2.28 (s, 3H). HRMS m/z [M+H]⁺ for C₁₈H₁₆N₃ calculated 274.1344, found 274.1342.

2-((1-Aminonaphthalen-2-yl)amino)isonicotinonitrile (11s): Following the procedure described for the synthesis of 11a, compound 10s (90 mg, 0.310 mmol) gave 11s as a greenish-yellow solid (50 mg, 62%, R_{f}=0.4, TLC eluent=5% methanol in CH₂Cl₂). ¹H NMR (400 MHZ, chloroform-d) δ 8.32 (dd, J=5.0, 0.9 Hz, 1H), 7.86 (tt, J=8.2, 3.3 Hz, 3H), 7.60-7.50 (m, 2H), 7.36 (d, J=8.6 Hz, 1H), 7.26 (d, J=8.6 Hz, 1H), 6.90 (dd, J=5.1, 1.4 Hz, 1H), 6.55 (s, 1H), 6.51 (d, J=1.1 Hz, 1H), 4.45 (s, 2H). HRMS m/z [M+H]⁺ for C₁₆H₁₂N₄ calculated 261.1140, found 261.1140.

5-((1-Aminonaphthalen-2-yl)amino) nicotinonitrile (11t): Following the procedure described for the synthesis of 11a, compound 10t (100 mg, 0.344 mmol) gave 11t as a greenish-yellow solid (50 mg, 56%, R_{f}=0.6, TLC eluent=5% methanol in CH₂Cl₂). ¹H NMR (400 MHZ, chloroform-d) § 8.38 (d, J=2.9 Hz, 1H), 8.31 (d, J=1.8 Hz, 1H), 7.91-7.80 (m, 2H), 7.60-7.50 (m, 2H), 7.36 (d, J=8.6 Hz, 1H), 7.21 (d, J=8.6 Hz, 1H), 7.02 (dd, J=2.8, 1.8 Hz, 1H), 5.62 (s, 1H), 4.40 (s, 2H). HRMS m/z [M+H]⁺ for C₁₆H₁₂N₄ calculated 261.1140, found 261.1143.

4-((1-Aminonaphthalen-2-yl)amino) picolinonitrile (11u): Following the procedure described for the synthesis of 11a, compound 10u (150 mg, 0.52 mmol) gave 11u as a yellowish solid (100 mg, 74%, R_{f}=0.3, TLC eluent=5% methanol in CH₂Cl₂). ¹H NMR (400 MHz, chloroform-d) δ 8.31 (d, J=5.7 Hz, 1H), 7.86 (dt, J=6.6, 3.6 Hz, 3H), 7.56 (dt, J=6.3, 3.3 Hz, 2H), 7.36 (d, J=8.6 Hz, 1H), 7.20 (d, J=8.6 Hz, 1H), 6.92 (d, J=2.4 Hz, 1H), 6.70 (dd, J=5.8, 2.4 Hz, 1H), 5.97 (s, 1H), 4.38 (s, 2H). HRMS m/z [M+H]⁺ for C₁₆H₁₂N₄ calculated 261.1140, found 261.1135.

6-((1-Aminonaphthalen-2-yl)amino) picolinonitrile (11v): Following the procedure described for the synthesis of 11a, compound 10v (140 mg, 0.48 mmol) gave 11v as a yellowish solid (60 mg, 48%, R_{f}=0.6, TLC eluent=100% CH₂Cl₂). ¹H NMR (400 MHZ, chloroform-d) δ 7.85 (ddt, J=7.9, 5.0, 3.1 Hz, 3H), 7.58-7.42 (m, 4H), 7.34 (d, J=8.6 Hz, 1H), 7.26 (d, J=9.4 Hz, 1H), 7.12 (d, J=7.2 Hz, 1H), 6.52 (d, J=8.6 Hz, 1H), 6.44 (s, 1H), 4.45 (s, 2H). HRMS m/z [M+H]⁺ for C₁₆H₁₂N₄ calculated 261.1140, found 261.1140.

Ethyl 3-((2-((3-cyanophenyl)amino) naphthalen-1-yl)amino)-3-oxopropanoate (12a): To an ice-cold solution of compound 11a (100 mg, 0.386 mmol) in THF (2 mL) was added triethylamine (81 μL, 0.578 mmol), followed by ethyl malonyl chloride (55 μL, 0.424 mmol) and the mixture stirred at room temperature for 3 h. the reaction was quenched by adding sat. NaHCO₃, and the product extracted in ethyl acetate. Organic layer dried over anhydrous sodium sulfate, evaporated, and the residue purified by silica gel column chromatography to afford 12a as a white solid (100 mg, 70%, R_{f}=0.6, TLC eluent=50% ethyl acetate in hexanes). ¹H NMR (400 MHZ, chloroform-d) δ 9.61 (s, 1H), 7.95 (d, J=8.5 Hz, 1H), 7.85 (d, J=8.1 Hz, 1H), 7.79 (d, J=8.9 Hz, 1H), 7.63-7.50 (m, 2H), 7.46 (ddd, J=8.1, 6.8, 1.1 Hz, 1H), 7.30 (t, J=7.9 Hz, 1H), 7.21 (t, J=1.9 Hz, 1H), 7.18-7.09 (m, 1H), 6.94 (s, 1H), 4.34 (q, J=7.1 Hz, 2H), 3.66 (s, 2H), 1.37 (t, J=7.1 Hz, 2H).

Ethyl 3-((2-((4-cyanopyridin-2-yl)amino) naphthalen-1-yl)amino)-3-oxopropanoate (12sa): Following the procedure described for the synthesis of 12a, compound 10s (50 mg, 0.192 mmol) gave 12sa as a white solid (55 mg, 76%, R_{f}=0.4, TLC eluent=50% ethyl acetate in hexanes). ¹H NMR (400 MHZ, chloroform-d) δ 9.54 (s, 1H), 8.29 (d, J=5.1 Hz, 1H), 7.96 (d, J=8.4 Hz, 1H), 7.90-7.74 (m, 4H), 7.58 (t, J=7.7 Hz, 1H), 7.49 (t, J=7.5 Hz, 1H), 6.91 (s, 1H), 6.88 (d, J=5.3 Hz, 1H), 4.35 (q, J=7.1 Hz, 2H), 3.68 (s, 2H), 1.38 (t, J=7.1 Hz, 3H). HRMS m/z [M+H]⁺ for C₂₁H₁₈N₄O₃ calculated 375.1457, found 375.1451.

3-(2,4-Dioxo-1,2,3,4-tetrahydro-5H-naphtho[1,2-b][1,4]diazepin-5-yl)benzonitrile (13a):
Method A: To a suspension of compound 11a (35 mg, 0.135 mmol) in anhydrous toluene (2 mL) at 0° C. was added malonyl chloride dropwise (15 μL, 0.162 mmol) with vigorous stirring. The reaction mixture was heated to 80° C. for 20 min and then at 110° C. for 10 min. The reaction mixture was cooled, added saturated NaHCO₃ solution and 5% iPrOH-CH₂Cl₂. Organic layer separated, dried over anhydrous Na₂SO₄, evaporated under reduced pressure. The residue was purified by silica gel chromatography to afford 13a as a white solid (18 mg, 40%, R_{f}=0.3, TLC eluent=50% ethyl acetate in hexanes). ¹H NMR (400 MHZ, chloroform-d) δ 8.61 (s, 1H), 8.10 (d, J=8.5 Hz, 1H), 7.89 (d, J=8.2 Hz, 1H), 7.73 (t, J=7.8 Hz, 1H), 7.68-7.57 (m, 4H), 7.53 (d, J=5.1 Hz, 2H), 6.93 (dd, J=8.9, 1.7 Hz, 1H), 3.63 (s, 2H). HRMS m/z [M+H]⁺ for C₂₀H₁₄N₃O₂ calculated 328.1086, found 328.1081.
Method B: To a solution of 12a (100 mg, 0.268 mmol) in THF (3 mL) was added a solution of potassium t-butoxide (1 M in THF, 0.3 mL, 0.295 mmol) and the mixture stirred at rt for 3 h. 1M aq. HCl was added, and the product was extracted in ethyl acetate. Organic layer separated, dried over anhydrous sodium sulfate, and the solvent was evaporated. The residue was purified by silica gel chromatography to afford 13a as light-yellow solid (90 mg, quantitative yield).
Method C: Compound 11a (100 mg, 0.386 mmol) was dissolved in a mixture of toluene (2 mL) and DMF (0.2 mL). Monomethyl malonate (45 μL, 0.424 mmol) and DCC (88 mg, 0.424 mmol) was added sequentially, and the mixture stirred at room temperature for 3 h (conversion to 12b was confirmed by TLC, R_{f}=0.6, eluent=50% ethyl acetate in hexanes). Potassium t-butoxide (1M in THF, 0.46 mL, 0.46 mmol) was added a stirred for additional 3 h at room temperature. 1M aq. HCl was added, and the product was extracted in ethyl acetate. Organic layer separated, dried over anhydrous sodium sulfate, and the solvent was evaporated. The residue was purified by silica gel chromatography to afford 13a (65 mg, 52%).

3-(10-Methyl-2,4-dioxo-1,2,3,4-tetrahydro-5H-naphtho[1,2-b][1,4]diazepin-5-yl)benzonitrile (13b): Following the procedure described for the synthesis of 13a (Method-A), compound 11b (420 mg, 1.53 mmol) gave 13b as a white solid (270 mg, 51%, R_{f}=0.3, TLC eluent=50% ethyl acetate in hexanes). ¹H NMR (400 MHz, chloroform-d) δ 8.60 (s, 1H), 7.88 (s, 1H), 7.78 (d, J=8.0 Hz, 1H), 7.59 (d, J=9.4 Hz, 3H), 7.53 (d, J=5.1 Hz, 2H), 7.47 (d, J=8.3 Hz, 1H), 6.85 (d, J=8.9 Hz, 1H), 3.62 (s, 2H), 2.61 (s, 3H). HRMS m/z [M+H]⁺ for C₂₁H₁₅N₃O₂ calculated 342.1243, found 342.1247.

3-(9-Methyl-2,4-dioxo-1,2,3,4-tetrahydro-5H-naphtho[1,2-b][1,4]diazepin-5-yl)benzonitrile (13c): Following the procedure described for the synthesis of 13a (Method-A), compound 11c (100 mg, 0.366 mmol) gave 13c as a white solid (85 mg, 68%, R_{f}=0.3, TLC eluent=50% ethyl acetate in hexanes). ¹H NMR (400 MHZ, chloroform-d) δ 9.34 (s, 1H), 8.06 (d, J=8.6 Hz, 1H), 7.69-7.45 (m, 6H), 6.88 (d, J=8.9 Hz, 1H), 3.63 (d, J=2.6 Hz, 2H), 2.55 (s, 3H). HRMS m/z [M+H]⁺ for C₂₁H₁₅N₃O₂ calculated 342.1243, found 342.1241.

3-(7-Bromo-2,4-dioxo-1,2,3,4-tetrahydro-5H-naphtho[1,2-b][1,4]diazepin-5-yl)benzonitrile (13d):
Method A: To a solution of 13a (10 mg, 0.031 mmol) in acetic acid (0.3 mL) was added bromine (8.0 μL, 0.155 mol) and the mixture stirred at 50° C. for 7 h. Volatile materials were evaporated under reduced pressure and the residue was treated with saturated NaHCO₃ and dichloromethane. Organic layer separated, dried over anhydrous Na₂SO₄. Solvent was evaporated and the residue was purified by silica gel column chromatography to afford 13d as a white solid (10 mg, 81%, R_{f}=0.3, TLC eluent=50% ethyl acetate in hexanes). ¹H NMR (400 MHz, chloroform-d) δ 9.72 (s, 1H), 8.26 (dd, J=15.8, 8.0 Hz, 2H), 7.77 (p, J=6.9 Hz, 2H), 7.71-7.61 (m, 2H), 7.57 (dd, J=13.8, 6.3 Hz, 2H), 7.28 (s, 1H), 3.65 (s, 2H). HRMS m/z [M+H]⁺ for C₂₀H₁₂N₃O₂Br calculated 406.0191, found 406.0186.
Method B: a solution of compound 7a (8 mg, 0.025 mmol) and NBS (22 mg, 0.125 mmol) in DMF was heated to 50° C. for 5 h. DMF was removed by rotary evaporation under high vacuum and the residue was portioned between saturated NaHCO₃ and dichloromethane. Organic layer separated, dried over anhydrous Na₂SO₄. Solvent was evaporated and the residue was purified by silica gel column chromatography to afford 13d as a white solid (8 mg, 81%).

3-(10-((Methylthio)methyl)-2,4-dioxo-1,2,3,4-tetrahydro-5H-naphtho[1,2-b][1,4]diazepin-5-yl)benzonitrile (13f): compound 14a (8.0 mg, 0.019 mmol) was dissolved in DMF (0.5 mL) and to this was added sodium thiomethoxide (4.0 mg, 0.057 mmol). After stirring the mixture at room temperature for 1 h, 1 mL each 1M HCl and water was added, and the product extracted in ethyl acetate. Organic layer was separated, dried over sodium sulfate, evaporated, and the residue dried under high vacuum. MS analysis showed only the desired product (13f) mass and carried forward to next step without purification (8.0 mg, quantitative, R_{f}=0.3, TLC eluent=50% ethyl acetate in hexanes). HRMS m/z [M+H]⁺ for C₂₂H₁₇N₃O₂S calculated 388.1120 found 388.1120.

Compounds 13e,g,h: 3-(10-(Azidomethyl)-2,4-dioxo-1,2,3,4-tetrahydro-5H-naphtho[1,2-b][1,4]diazepin-5-yl)benzonitrile (13g): Compound 14a (along with 14b and 13e, 40 mg, 0.095 mmol) was dissolved in DMF and added NaN₃ (62 mg, 0.95 mmol). The reaction mixture was heated to 50° C. for 18 h. The solvent was rotary evaporated under high vacuum and the residue was partitioned between water and ethyl acetate. Organic layer was separated, dried, and evaporated. The residue was purified by silica gel flash chromatography to afford 13g (30 mg, 82%, R_{f}=0.25, TLC eluent=50% ethyl acetate in hexanes). ¹H NMR (400 MHZ, chloroform-d) δ 8.95 (s, 1H), 8.09 (s, 1H), 7.91 (d, J=8.4 Hz, 1H), 7.70-7.48 (m, 5H), 6.95 (d, J=9.0 Hz, 1H), 4.62 (s, 2H), 3.64 (s, 2H). MS m/z [M+H]⁺ for C₂₁H₁₄N₆O₂ calculated 383.1251 found 383.1. Compound 13e+13h could be separated in this step (R_{f}=0.30, TLC eluent=50% ethyl acetate in hexanes). ¹H NMR (400 MHZ, chloroform-d) δ 9.17 (s, 1H), 8.70 (s, 1H), 8.29 (d, J=8.6 Hz, OH), 8.21-8.11 (m, 1H), 7.91 (s, 1H), 7.73-7.37 (m, 7H), 7.31-7.27 (m, 1H), 7.16 (s, 1H), 4.64 (s, 1H, naphthalene-7-CH₂N₃ of 13h), 3.64 (s, 1H, 1,4-diazepine-CH₂ of 13h), 3.62 (s, 2H, 1,4-diazepine-CH₂ of 13e), 2.63 (s, 3H, naphthalene-7-Me of 13e). MS for 13e; m/z [M+H]⁺ for C₂₁H₁₄N₃O₂Br calculated 420.0342; 422.0322 found 420.0; 422.0. MS for 13h; m/z [M+H]⁺ for C₂₁H₁₃N₆O₂Br calculated 461.0356; 463.0336 found 461.0; 463.0.

N-((5-(3-Cyanophenyl)-2,4-dioxo-2,3,4,5-tetrahydro-1H-naphtho[1,2-b][1,4]diazepin-10-yl)methyl) acetamide (13i): The azido intermediate 13g (25 mg, 0.065 mmol) was dissolved in 9:1 THF-H₂O (1 mL) and to this was added trimethyl phosphine (PMe₃, 20 μL, 0.196 mmol). After stirring the reaction mixture at room temperature for 5 h, volatiles were rotary evaporated. The residue 15 was dried under high vacuum (25 mg, R_{f}=0.35, TLC eluent=20% methanol in dichloromethane). To the intermediate 15 (0.065 mmol) in THF (2 mL) was added triethylamine (18 μL, 0.130 mmol), acetic anhydride (13 μL, 0.130 mmol) and the mixture stirred at room temperature for 18 h. Water was added to the reaction mixture and the product was extracted several times in ethyl acetate. The combined organic layers were dried over sodium sulfate, filtered, filtrate evaporated to afford 13i and used as such in next step without purification/characterization (25 mg, 89%, R_{f}=0.35, TLC eluent=5% methanol in dichloromethane).

Compounds 13j and 13k: 3-(9-(Azidomethyl)-2,4-dioxo-1,2,3,4-tetrahydro-5H-naphtho[1,2-b][1,4]diazepin-5-yl)benzonitrile (13j): Following the method described for the synthesis of 13g, compound 16a,b (25 mg, 0.060 mmol) gave 13j as a major product (10 mg, 44%, R_{f}=0.3, TLC eluent=50% ethyl acetate in hexanes). ¹H NMR (400 MHZ, chloroform-d) δ 9.22 (s, 1H), 8.19 (d, J=8.6 Hz, 1H), 7.82 (s, 1H), 7.73-7.40 (m, 5H), 6.96 (d, J=8.9 Hz, 1H), 4.57 (s, 2H), 4.62 (s, 0.3H, naphthalene-6-CH₂N₃ of 13k), 3.64 (s, 2H). HRMS m/z [M+H]⁺ for C₂₁H₁₄N₆O₂ calculated 383.1256 found 383.1255.

3-(2,4-Dioxo-7-phenyl-1,2,3,4-tetrahydro-5H-naphtho[1,2-b][1,4]diazepin-5-yl)benzonitrile (13l): To a round bottom flask equipped with stir bar was added 13d (30 mg, 0.074 mmol), phenylboronic acid (14 mg, 0.111 mmol), Pd(PPh₃)₄ (9 mg, 0.008 mmol), sodium carbonate (23 mg, 0.222 mmol) and subjected to few vacuum-Argon degassing cycles. To the above mixture was added 1,2-dimethoxyethane (2 mL), water (0.2 mL) and heated to 90° C. for 5 h. The solvent was evaporated, and the residue was partitioned between water and dichloromethane. Organic layer was separated, dried over anhydrous sodium sulfate, filtered, filtrate evaporated. The residue was purified by silica gel flash chromatography to obtain 13l as a white solid (23 mg, 77%, R_{f}=0.3, TLC eluent=50% ethyl acetate in hexanes). ¹H NMR (400 MHz, chloroform-d) δ 8.97 (s, 1H), 8.22 (d, J=8.4 Hz, 1H), 7.95-7.88 (m, 1H), 7.76 (ddd, J=8.4, 6.9, 1.3 Hz, 1H), 7.67-7.40 (m, 4H), 7.31 (dd, J=7.2, 2.2 Hz, 2H), 6.88 (s, 1H), 3.82-3.56 (m, 2H). HRMS m/z [M+H]⁺ for C₂₆H₁₇N₃O₂ calculated 404.1399, found 404.1399.

3-(2,4-Dioxo-7-(phenylethynyl)-1,2,3,4-tetrahydro-5H-naphtho[1,2-b][1,4]diazepin-5-yl)benzonitrile (13m): Following the protocol described for the synthesis of 17, 13d (25 mg, 0.062 mmol) gave 13m as a light brown solid (17 mg, 65%, R_{f}=0.3, TLC eluent=50% ethyl acetate in hexanes). ¹H NMR (400 MHZ, chloroform-d) δ 9.35 (d, J=4.3 Hz, 1H), 8.29 (dd, J=8.3, 1.5 Hz, 1H), 8.21 (dd, J=8.0, 1.6 Hz, 2H), 7.79 (dddd, J=16.5, 12.2, 6.7, 2.3 Hz, 3H), 7.71-7.50 (m, 4H), 7.45-7.35 (m, 2H), 7.26 (s, 1H), 3.65 (s, 2H). HRMS m/z [M+H]⁺ for C₂₈H₁₇N₃O₂ calculated 428.1399 found 428.1405.

3-(2,4-Dioxo-7-(pyridin-3-ylethynyl)-1,2,3,4-tetrahydro-5H-naphtho[1,2-b][1,4]diazepin-5-yl)benzonitrile (13n): Following the protocol described for the synthesis of 17, 13d (20 mg, 0.049 mmol) gave 13n as a light brown solid (17 mg, 80%, R_{f}=0.5, TLC eluent=ethyl acetate). ¹H NMR (400 MHZ, chloroform-d) δ 9.43 (s, 1H), 8.88-8.80 (m, 1H), 8.61 (dd, J=5.0, 1.7 Hz, 1H), 8.47-8.39 (m, 1H), 8.30-8.20 (m, 1H), 7.89 (dt, J=7.9, 1.9 Hz, 1H), 7.82-7.73 (m, 2H), 7.60 (d, J=1.9 Hz, 1H), 7.35 (dd, J=7.9, 4.9 Hz, 1H), 7.21 (s, 1H), 3.65 (s, 2H). HRMS m/z [M+H]⁺ for C₂₇H₁₆N₄O₂ calculated 429.1372 found 429.1351.

3-(7-(Hex-1-yn-1-yl)-2,4-dioxo-1,2,3,4-tetrahydro-5H-naphtho[1,2-b][1,4]diazepin-5-yl)benzonitrile (13o): Following the protocol described for the synthesis of 17, 13d (20 mg, 0.05 mmol) gave 13o as a light brown solid (10 mg, 50%, R_{f}=0.4, TLC eluent=50% ethyl acetate in hexanes). ¹H NMR (400 MHZ, chloroform-d) δ 9.55 (s, 1H), 8.37 (dd, J=8.0, 1.5 Hz, 1H), 8.24-8.15 (m, 1H), 7.73 (dddd, J=19.1, 8.1, 6.9, 1.4 Hz, 2H), 7.64 (dq, J=4.5, 1.6 Hz, 1H), 7.61-7.48 (m, 2H), 7.05 (s, 1H), 3.64 (s, 2H), 2.52 (td, J=7.2, 2.5 Hz, 2H), 1.73-1.60 (m, 2H), 1.59-1.42 (m, 2H), 0.97 (t, J=7.3 Hz, 3H). HRMS m/z [M+H]⁺ for C₂₆H₂₁N₃O₂ calculated 408.1712 found 408.1707.

3-(2,4-Dioxo-7-((trimethylsilyl) ethynyl)-1,2,3,4-tetrahydro-5H-naphtho[1,2-b][1,4]diazepin-5-yl)benzonitrile (17): In a round bottom flask equipped with stir bar was charged with 13d (30 mg, 0.074 mmol), triphenylphosphine (20 mg, 0.074 mmol), Pd(PPh₃)₂Cl₂ (10 mg, 0.016 mmol), copper (1) iodide (CuI, 6 mg, 0.030 mmol) and subjected to few vacuum-Argon degassing cycles. To the above mixture was added 1,2-dimethoxyethane (1 mL), triethylamine (0.25 mL) and trimethylsilylacetylene (21 μL, 0.148 mmol) sequentially, and heated to 55° C. for 6 h [Note: 5-6 equivalents of volatile alkynes and 6-18 h heating may be needed]. To the reaction mixture was added silica gel, and the solvent was evaporated to load on it, and purified by silica gel flash chromatography to obtain 17 as a white solid (25 mg, 80%, R_{f}=0.4, TLC eluent=50% ethyl acetate in hexanes). ¹H NMR (400 MHz, chloroform-d) δ 9.80 (s, 1H), 8.42-8.30 (m, 1H), 8.22 (dd, J=7.8, 1.5 Hz, 1H), 7.74 (dddd, J=16.1, 8.2, 7.0, 1.4 Hz, 2H), 7.64 (dt, J=8.7, 1.5 Hz, 2H), 7.59-7.48 (m, 2H), 7.12 (s, 1H), 3.62 (d, J=1.4 Hz, 2H), 0.30 (s, 9H). HRMS m/z [M+H]⁺ for C₂₅H₂₁N₃O₂Si calculated 424.1481 found 424.1479.

Compounds 18, 19:3-(7-Ethynyl-2,4-dioxo-1,2,3,4-tetrahydro-5H-naphtho[1,2-b][1,4]diazepin-5-yl)benzonitrile (19): To a solution of compound 17 (with traces of 18, 25 mg, 0.059 mmol) was added TBAF (1M in THF, 90 μL, 0.089 mmol) and the mixture stirred at room temperature for 18 h. Silica gel was added, subjected to rotary evaporation to load on it and purified by flash column chromatography to afford 18 and 19 as a white solid. Data for 19 (13 mg, 63%, R_{f}=0.3, TLC eluent=50% ethyl acetate in hexanes). ¹H NMR (400 MHZ, chloroform-d) δ 9.20 (s, 1H), 8.46-8.37 (m, 1H), 8.19 (dd, J=7.8, 1.4 Hz, 1H), 7.77 (dddd, J=16.6, 8.2, 6.9, 1.4 Hz, 2H), 7.70-7.49 (m, 3H), 7.19 (s, 1H), 3.65 (d, J=1.4 Hz, 2H), 3.49 (s, 1H). HRMS m/z [M+H]⁺ for C₂₂H₁₃N₃O₂ calculated 352.1086 found 352.1087. Data for 18 (3 mg, 12%, R_{f}=0.4, TLC eluent=50% ethyl acetate in hexanes, kst4059a): ¹H NMR (400 MHZ, chloroform-d) δ 8.80 (s, 1H), 8.23-8.11 (m, 1H), 8.08-7.94 (m, 1H), 7.84-7.47 (m, 4H), 6.76 (s, 1H), 3.63 (t, J=2.4 Hz, 2H), 3.03-2.82 (m, 2H), 0.94-0.70 (m, 2H), 0.02 (s, 9H). HRMS m/z [M+H]⁺ for C₂₅H₂₅N₃O₂Si calculated 428.1794 found 428.1792.

3-(7-Ethyl-2,4-dioxo-1,2,3,4-tetrahydro-5H-naphtho[1,2-b][1,4]diazepin-5-yl)benzonitrile (13p): Compound 19 (13 mg, 0.037 mmol) was dissolved ethyl acetate (1.5 mL) and to this was added 10% Pd—C(15 mg). A stream of hydrogen gas was bubbled through the reaction mixture for 5 h. the palladium catalyst was filtered, filtrate concentrated, and the residue was purified by silica gel flash column chromatography to afford 13p as white solid (8 mg, 61%, R_{f}=0.3, TLC eluent=50% ethyl acetate in hexanes). ¹H NMR (400 MHZ, chloroform-d) δ 9.31 (s, 1H), 8.26-8.15 (m, 1H), 8.08 (dd, J=8.3, 1.4 Hz, 1H), 7.77-7.58 (m, 3H), 7.58-7.49 (m, 2H), 6.75 (s, 1H), 3.72-3.56 (m, 2H), 2.98 (dp, J=22.4, 7.4 Hz, 2H), 1.38-1.13 (m, 3H). HRMS m/z [M+H]⁺ for C₂₂H₁₇N₃O₂ calculated 356.1399 found 356.1396.

3-(2,4-Dioxo-1,2,3,4-tetrahydro-5H-naphtho[1,2-b][1,4]diazepin-5-yl)-5-methylbenzonitrile (13r): Following the procedure described for the synthesis of 13a (Method-A), compound 11r (51 mg, 0.187 mmol) gave 13r as a yellowish solid (34 mg, 54%, R_{f}=0.3, TLC eluent=50% ethyl acetate in hexanes). ¹H NMR (400 MHZ, chloroform-d) δ 8.71 (s, 1H), 8.13 (d, J=8.5 Hz, 1H), 7.90 (d, J=8.0 Hz, 1H), 7.74 (ddd, J=8.5, 7.0, 1.4 Hz, 1H), 7.71-7.60 (m, 2H), 7.46-7.39 (m, 1H), 7.35 (s, 1H), 6.96 (d, J=9.0 Hz, 1H), 3.64 (s, 2H), 2.40 (s, 3H). HRMS m/z [M+H]⁺ for C₂₁H₁₆N₃O₂ calculated 342.1243, found 342.1246.

2-(2,4-Dioxo-1,2,3,4-tetrahydro-5H-naphtho[1,2-b][1,4]diazepin-5-yl)isonicotinonitrile (13s): Following the procedure described for the synthesis of 13a (Method-A), compound 11s (30 mg, 0.103 mmol) gave 13s as a light brown solid (12 mg, 35%, R_{f}=0.3, TLC eluent=50% ethyl acetate in hexanes). ¹H NMR (400 MHZ, chloroform-d) δ 8.77 (s, 1H), 8.64-8.56 (m, 1H), 8.17-8.08 (m, 2H), 7.95-7.86 (m, 1H), 7.77-7.61 (m, 3H), 7.47 (dd, J=5.0, 1.4 Hz, 1H), 6.90 (d, J=9.0 Hz, 1H), 3.72 (d, J=12.0 Hz, 1H), 3.64 (dd, J=11.9, 1.7 Hz, 1H). HRMS m/z [M+H]⁺ for C₁₉H₁₃N₄O₂ calculated 329.1039, found 329.1035.

5-(2,4-Dioxo-1,2,3,4-tetrahydro-5H-naphtho[1,2-b][1,4]diazepin-5-yl) nicotinonitrile (13t): Compound 11t (50 mg, 0.172 mmol) was dissolved in a mixture of anhydrous toluene-dioxane (2:1, 5 mL) and at rt was added malonyl chloride (25 μL, 0.26 mmol). The reaction mixture was heated to 110° C. for 30 min under reflux condenser with vigorous stirring. The solvents were evaporated under reduced pressure, and the residue was partitioned between dilute aq. NaHCO₃ solution and ethyl acetate. The product was extracted with ethyl acetate repeatedly until organic layer showed no product in TLC. Organic layers were combined, dried, evaporated to give 13t as a light brown solid (30 mg, 53%, R_{f}=0.1, TLC eluent=50% ethyl acetate in hexanes). ¹H NMR (400 MHZ, chloroform-d) δ 8.81 (d, J=1.9 Hz, OH), 8.69 (d, J=2.5 Hz, OH), 8.34 (s, 1H), 8.10 (d, J=8.4 Hz, 1H), 8.03 (t, J=2.1 Hz, OH), 7.94 (d, J=8.0 Hz, 1H), 7.77 (ddd, J=8.4, 6.9, 1.4 Hz, 1H), 7.74-7.65 (m, 2H), 6.93 (d, J=8.9 Hz, 1H), 3.68 (s, 2H). HRMS m/z [M+H]⁺ for C₁₉H₁₃N₄O₂ calculated 329.1039, found 329.1040.

4-(2,4-Dioxo-1,2,3,4-tetrahydro-5H-naphtho[1,2-b][1,4]diazepin-5-yl) picolinonitrile (13u): Following the procedure described for the synthesis of 13t, compound 11u (100 mg, 0.38 mmol) gave 13u as a light-yellow solid (30 mg, 24%, R_{f}=0.3, TLC eluent=50% ethyl acetate in hexanes). ¹H NMR (400 MHZ, chloroform-d) δ 8.94 (s, 1H), 8.72 (d, J=5.4 Hz, 1H), 8.16 (d, J=8.4 Hz, 1H), 7.95 (dd, J=8.1, 1.3 Hz, 1H), 7.83-7.63 (m, 4H), 7.52 (dd, J=5.5, 2.2 Hz, 1H), 6.97 (d, J=8.9 Hz, 1H), 3.76-3.60 (m, 2H). HRMS m/z [M+H]⁺ for C₁₉H₁₃N₄O₂ calculated 329.1039, found 329.1038.

6-(2,4-Dioxo-1,2,3,4-tetrahydro-5H-naphtho[1,2-b][1,4]diazepin-5-yl) picolinonitrile (13v): Following the procedure described for the synthesis of 13t, compound 11v (60 mg, 0.23 mmol) gave 13v as a light-yellow solid (25 mg, 33%, R_{f}=0.1, TLC eluent=50% ethyl acetate in hexanes). ¹H NMR (400 MHZ, chloroform-d) δ 8.28 (s, 1H), 8.04 (ddd, J=21.6, 15.4, 8.3 Hz, 3H), 7.92 (d, J=8.1 Hz, 1H), 7.76-7.62 (m, 4H), 6.91 (d, J=8.9 Hz, 1H), 3.71 (d, J=12.0 Hz, 1H), 3.62 (dd, J=11.9, 1.8 Hz, 1H). HRMS m/z [M+H]⁺ for C₁₉H₁₃N₄O₂ calculated 329.1039, found 329.1034.

3-(10-(Bromomethyl)-2,4-dioxo-1,2,3,4-tetrahydro-5H-naphtho[1,2-b][1,4]diazepin-5-yl)benzonitrile (14a): A mixture of compound 13b (50 mg, 0.146 mmol), N-bromosuccinimide (NBS, 32 mg, 0.176 mmol) and benzoyl peroxide (36 mg, 0.148 mmol) in benzene (2 mL) was heated to 85° C. for 18 h. to the reaction mixture was added dichloromethane, water and a pinch of sodium bisulfite. Organic layer was separated, aqueous layer was extracted several times with dichloromethane. Combined organic layer was dried over anhydrous sodium sulfate and rotary evaporated. The residue was purified by silica gel column chromatography to afford mixture of products 14a,b, and 7e (40 mg, 65%, R_{f}=0.3, TLC eluent=50% ethyl acetate in hexanes). NMR analysis showed 14a as a major product. ¹H NMR (400 MHz, chloroform-d) δ 9.08 (s, 1H), 8.18 (s, 1H), 7.89 (d, J=8.4 Hz, 1H), 7.71-7.51 (m, 6H), 6.95 (d, J=9.0 Hz, 1H), 4.73 (d, J=3.1 Hz, 2H), 3.64 (d, J=5.3 Hz, 3H). MS m/z [M+H]⁺ for C₂₁H₁₄N₃O₂Br calculated 420.0342, 422.0322, found 420.0, 422.0. Mass analysis of the mixture showed presence of 14b. MS m/z [M+H]⁺ for C₂₁H₁₃N₃O₂Br2 calculated 499.9427, found 499.9.

3-(9-(Bromomethyl)-2,4-dioxo-1,2,3,4-tetrahydro-5H-naphtho[1,2-b][1,4]diazepin-5-yl)benzonitrile (16a): following the procedure described for the synthesis of 14a, using 13c (50 mg, 0.146 mmol) and dichloroethane as solvent, gave the desired product mixtures 16a,b as a light brown solid (25 mg, 41%, R_{f}=0.3, TLC eluent=50% ethyl acetate in hexanes). NMR and mass analysis showed 16a as a major product. ¹H NMR (400 MHZ, chloroform-d) δ 10.01 (s, 1H), 8.23 (d, J=8.6 Hz, 1H), 7.85 (s, 1H), 7.75-7.43 (m, 6H), 6.94 (d, J=9.0 Hz, 1H), 4.65 (s, 2H), 3.61 (s, 2H). HRMS m/z [M+H]⁺ for C₂₁H₁₄N₃O₂Br calculated 420.0348, found 420.0349.

(Z)-3-(2,4-Dioxo-1,2,3,4-tetrahydro-5H-naphtho[1,2-b][1,4]diazepin-5-yl)-N′-hydroxybenzimidamide (20a): To compound 13a (75 mg, 0.229 mmol) in THF-MeOH (1:2) was added hydroxylamine hydrochloride (160 mg, 2.29 mmol) followed by triethylamine (0.32 mL, 2.29 mmol) and heated to 70° C. for 2 h under reflux condenser. Solvents were evaporated and residue triturated with water. The precipitates were collected by filtration and dried to afford 20a as a yellowish solid and used as such to next step without further purification (75 mg, 91%, R_{f}=0.3, TLC eluent=100% ethyl acetate). HRMS m/z [M+H]⁺ for C₂₀H₁₆N₄O₃ calculated 361.1301, found 361.1296.

(Z)—N′-Hydroxy-3-(10-methyl-2,4-dioxo-1,2,3,4-tetrahydro-5H-naphtho[1,2-b][1,4]diazepin-5-yl)benzimidamide (20b): Following the procedure described for the synthesis of 20a, compound 13b (14 mg, 0.041 mmol) gave 20b as a whitish solid (14 mg, 91%, R_{f}=0.3, TLC eluent=100% ethyl acetate). HRMS m/z [M+H]⁺ for C₂₁H₁₈N₄O₃ calculated 375.1457, found 375.1451.

(Z)—N′-Hydroxy-3-(9-methyl-2,4-dioxo-1,2,3,4-tetrahydro-5H-naphtho[1,2-b][1,4]diazepin-5-yl)benzimidamide (20c): Following the procedure described for the synthesis of 20a, compound 13c (21 mg, 0.062 mmol) gave 20c as a whitish solid (17 mg, 74%, R_{f}=0.3, TLC eluent=100% ethyl acetate). HRMS m/z [M+H]⁺ for C₂₁H₁₈N₄O₃ calculated 375.1457, found 375.1463.

(Z)-3-(7-bromo-2,4-dioxo-1,2,3,4-tetrahydro-5H-naphtho[1,2-b][1,4]diazepin-5-yl)-N′-hydroxybenzimidamide (20d-kst4042): Following the procedure described for the synthesis of 20a, compound 13d (28 mg, 0.069 mmol) gave 20d as a whitish solid (18 mg, 59%, R_{f}=0.3, TLC eluent=100% ethyl acetate). HRMS m/z [M+H]⁺ for C₂₀H₁₅N₄O₃Br calculated 439.0406, found 439.0399.

(Z)-3-(7-Bromo-10-methyl-2,4-dioxo-1,2,3,4-tetrahydro-5H-naphtho[1,2-b][1,4]diazepin-5-yl)-N′-hydroxybenzimidamide (20e): See 20g.

(Z)—N′-Hydroxy-3-(10-((methylthio)methyl)-2,4-dioxo-1,2,3,4-tetrahydro-5H-naphtho[1,2-b][1,4]diazepin-5-yl)benzimidamide (20f): To compound 13f (8 mg, 0.021 mmol) in THF-MeOH (1:1) was added hydroxylamine hydrochloride (15 mg, 0.21 mmol) followed by triethylamine (30 μL, 0.21 mmol) and heated to 70° C. for 2 h under a reflux condenser. Water was added, product extracted in ethyl acetate several times (till UV spots were absent on TLC). The combined organic layers were dried over anhydrous sodium sulfate, filtered, filtrate was concentrated. The residue was dried under high vacuum to afford 20f as a brownish solid and used as such to next step without further purification/characterization (7 mg, 81%, R_{f}=0.3, TLC eluent=100% ethyl acetate).

(Z)-3-(10-(Azidomethyl)-2,4-dioxo-1,2,3,4-tetrahydro-5H-naphtho[1,2-b][1,4]diazepin-5-yl)-N′-hydroxybenzimidamide (20g): Following the procedure described for the synthesis of 20f, compound 13g (as mixture of 13e and 13 h, 30 mg, 0.078 mmol) gave 20g as a brown solid (35 mg, quantitative, R_{f}=0.3, TLC eluent=100% ethyl acetate). Data for 20g: HRMS m/z [M+H]⁺ for C₂₁H₁₇N₇O₃ calculated 416.1471, found 416.1474.

(Z)—N-((5-(3-(N′-Hydroxycarbamimidoyl)phenyl)-2,4-dioxo-2,3,4,5-tetrahydro-1H-naphtho[1,2-b][1,4]diazepin-10-yl)methyl) acetamide (20i): Following the procedure described for the synthesis of 20f, compound 13i (25 mg, 0.065 mmol) gave 20i as a brown solid (25 mg, quantitative, R_{f}=0.3, TLC eluent=10% methanol in dichloromethane). HRMS m/z [M+H]⁺ for C₂₃H₂₁N₅O₄ calculated 432.1672, found 432.1679.

(Z)-3-(9-(Azidomethyl)-2,4-dioxo-1,2,3,4-tetrahydro-5H-naphtho[1,2-b][1,4]diazepin-5-yl)-N′-hydroxybenzimidamide (20j): Following the procedure described for the synthesis of 20f, compound 13j (as a mixture with 13k, 13 mg, 0.034 mmol) gave 20j as a brown solid (15 mg, quantitative, R_{f}=0.4, TLC eluent=100% ethyl acetate). HRMS m/z [M+H]⁺ for C₂₁H₁₇N₇O₃ calculated 416.1471, found 416.1468. Data for 20k: MS m/z [M+H]⁺ for C₂₁H₁₆N₇O₃Br calculated 494.0571, found 494.1, 496.1.

(Z)-3-(2,4-Dioxo-7-phenyl-1,2,3,4-tetrahydro-5H-naphtho[1,2-b][1,4]diazepin-5-yl)-N′-hydroxybenzimidamide (20l): Following the procedure described for the synthesis of 20f, compound 13l (23 mg, 0.057 mmol) gave 20l as a brown solid (17 mg, 68%, R_{f}=0.3, TLC eluent=100% ethyl acetate). HRMS m/z [M+H]⁺ for C₂₆H₂₀N₄O₃ calculated 437.161, found 437.1615.

(Z)-3-(2,4-Dioxo-7-(phenylethynyl)-1,2,3,4-tetrahydro-5H-naphtho[1,2-b][1,4]diazepin-5-yl)-N′-hydroxybenzimidamide (20m): Following the procedure described for the synthesis of 20f, compound 13m (18 mg, 0.04 mmol) gave 20m as a brown solid (17 mg, 93%, R_{f}=0.3, TLC eluent=ethyl acetate). HRMS m/z [M+H]⁺ for C₂₈H₂₀N₄O₃ calculated 461.1614, found 461.1614.

(Z)-3-(2,4-Dioxo-7-(pyridin-3-ylethynyl)-1,2,3,4-tetrahydro-5H-naphtho[1,2-b][1,4]diazepin-5-yl)-N′-hydroxybenzimidamide (20n): Following the procedure described for the synthesis of 20f, compound 13n (16 mg, 0.037 mmol) gave 20n as a brown solid (15 mg, 93%, R_{f}=0.1, TLC eluent=ethyl acetate). HRMS m/z [M+H]⁺ for C₂₇H₁₉N₅O₃ calculated 462.1566, found 462.1560.

(Z)-3-(7-(Hex-1-yn-1-yl)-2,4-dioxo-1,2,3,4-tetrahydro-5H-naphtho[1,2-b][1,4]diazepin-5-yl)-N′-hydroxybenzimidamide (20o): Following the procedure described for the synthesis of 20f, compound 13o (10 mg, 0.023 mmol) gave 20o as a brown solid (12 mg, quantitative, R_{f}=0.2, TLC eluent=ethyl acetate). HRMS m/z [M+H]⁺ for C₂₆H₂₄N₄O₃ calculated 441.1927, found 441.1923.

(Z)-3-(7-Ethyl-2,4-dioxo-1,2,3,4-tetrahydro-5H-naphtho[1,2-b][1,4]diazepin-5-yl)-N′-hydroxybenzimidamide (20p): Following the procedure described for the synthesis of 20f, compound 13p (8 mg, 0.023 mmol) gave 20p as a brown solid (10 mg, quantitative, R_{f}=0.3, TLC eluent=100% ethyl acetate). HRMS m/z [M+H]⁺ for C₂₂H₂₀N₄O₃ calculated 389.1614, found 389.1617.

(Z)-3-(2,4-Dioxo-1,2,3,4-tetrahydro-5H-naphtho[1,2-b][1,4]diazepin-5-yl)-N′-hydroxy-5-methylbenzimidamide (20r): Following the procedure described for the synthesis of 20a, compound 13r (30 mg, 0.088 mmol) gave 20r as a light brown solid (34 mg, quantitative, R_{f}=0.3, TLC eluent=ethyl acetate). HRMS m/z [M+H]⁺ for C₂₁H₁₈N₄O₃ calculated 375.1457, found 375.1459.

(Z)-2-(2,4-Dioxo-1,2,3,4-tetrahydro-5H-naphtho[1,2-b][1,4]diazepin-5-yl)-N′-hydroxyisonicotinimidamide (20s): Compound 13s (30 mg, 0.092 mmol) was dissolved in THF-MeOH (1:1 v/v, 3 mL) and was added hydroxylamine hydrochloride (13 mg, 0.184 mmol) followed by triethylamine (26 μL, 0.184 mmol), and the mixture was stirred at room temperature for 1-2 h (or reaction completion as indicated by TLC). The volatiles were evaporated under reduced pressure and the residue was partitioned between water-ethyl acetate. Product was extracted repeatedly with ethyl acetate (4-5 times, or as indicated by TLC), combined, and the solvent was evaporated and dried under high vacuum to afford 20s as light brown solid (25 mg, 75%, R_{f}=0.2, TLC eluent=ethyl acetate). HRMS m/z [M+H]⁺ for C₁₉H₁₅N₅O₃ calculated 362.1253, found 362.1255.

(Z)-5-(2,4-Dioxo-1,2,3,4-tetrahydro-5H-naphtho[1,2-b][1,4]diazepin-5-yl)-N′-hydroxynicotinimidamide (20t): Following the procedure described for the synthesis of 20s, compound 13t (30 mg, 0.092 mmol) gave 20t as a light brown solid (25 mg, 75%, R_{f}=0.3, TLC eluent=ethyl acetate).

(Z)-4-(2,4-Dioxo-1,2,3,4-tetrahydro-5H-naphtho[1,2-b][1,4]diazepin-5-yl)-N′-hydroxypicolinimidamide (20u): Following the procedure described for the synthesis of 20s, compound 13u (30 mg, 0.092 mmol) gave 20u as a light brown solid (25 mg, 75%, R_{f}=0.3, TLC eluent=ethyl acetate). HRMS m/z [M+H]⁺ for C₁₉H₁₅N₅O₃ calculated 362.1253, found 362.1255.

(Z)-6-(2,4-Dioxo-1,2,3,4-tetrahydro-5H-naphtho[1,2-b][1,4]diazepin-5-yl)-N′-hydroxypicolinimidamide (20v): Following the procedure described for the synthesis of 20s, compound 13v (25 mg, 0.076 mmol) gave 20v as a light brown solid (25 mg, 91%, R_{f}=0.5, TLC eluent=ethyl acetate). HRMS m/z [M+H]⁺ for C₁₉H₁₅N₅O₃ calculated 362.1253, found 362.1256.

5-(3-(5-Thioxo-4,5-dihydro-1,2,4-oxadiazol-3-yl)phenyl)-1,5-dihydro-2H-naphtho[1,2-b][1,4]diazepine-2,4(3H)-dione (NP-1815-PX; 21a): Compound 20a (75 mg, 0.21 mmol) was suspended in anhydrous acetonitrile (3 mL) and to this at 0° C. was added DBU (125 μL, 0.84 mmol) and the mixture stirred for 15 min. Thiocarbonyldiimidazole (56 mg, 0.32 mmol) was added and the mixture stirred at room temperature for 1.5 h. Dichloromethane and 1M HCl was added, layer separated and the aqueous layer was extracted several times with 5% isopropanol in dichloromethane. The combined organic layer was dried over anhydrous sodium sulfate, filtered, filtrate concentrated, and the residue was purified by silica gel flash column chromatography to afford 21a as white/yellowish solid. The compound may be purified further by preparative-TLC (35 mg, 42%, R_{f}=0.35, TLC eluent=15% methanol in dichloromethane). ¹H NMR (400 MHZ, DMSO-d₆) δ 10.90 (s, 1H), 8.24 (d, J=8.5 Hz, 1H), 7.92 (d, J=8.0 Hz, 1H), 7.83 (d, J=7.7 Hz, 1H), 7.74-7.64 (m, 2H), 7.64-7.57 (m, 1H), 7.54 (t, J=7.9 Hz, 1H), 7.41-7.33 (m, 1H), 7.02 (d, J=9.0 Hz, 1H), 3.75 (d, J=11.9 Hz, 1H), 3.17 (d, J=11.9 Hz, 1H). HRMS m/z [M+H]⁺ for C₂₁H₁₄N₄O₃S calculated 403.0865, found 403.0866. Purity—98.74% at 254 nm.

10-Methyl-5-(3-(5-thioxo-4,5-dihydro-1,2,4-oxadiazol-3-yl)phenyl)-1,5-dihydro-2H-naphtho[1,2-b][1,4]diazepine-2,4(3H)-dione (21b; MRS4595): Following the procedure described for the synthesis of 21a, compound 20b (14 mg, 0.037 mmol) gave 21b as a white/yellowish solid (7 mg, 45%, R_{f}=0.35, TLC eluent=15% methanol in dichloromethane). ¹H NMR (400 MHZ, DMSO-d₆) δ 10.81 (s, 1H), 8.05 (s, 1H), 7.83 (dd, J=8.0, 5.6 Hz, 2H), 7.65 (d, J=8.7 Hz, 2H), 7.58 (t, J=7.9 Hz, 1H), 7.43 (dd, J=14.3, 7.6 Hz, 2H), 6.93 (d, J=9.0 Hz, 1H), 3.73 (d, J=11.8 Hz, 1H), 3.16 (d, J=11.8 Hz, 1H), 2.54 (s, 3H). HRMS m/z [M+H]⁺ for C₂₂H₁₆N₄O₃S calculated 417.1021, found 417.1025. Purity—96.68% at 254 nm.

9-Methyl-5-(3-(5-thioxo-4,5-dihydro-1,2,4-oxadiazol-3-yl)phenyl)-1,5-dihydro-2H-naphtho[1,2-b][1,4]diazepine-2,4(3H)-dione (21c; MRS4596): Following the procedure described for the synthesis of 21a, compound 20c (17 mg, 0.045 mmol) gave 21c as a white/yellowish solid (10 mg, 53%, R_{f}=0.35, TLC eluent=15% methanol in dichloromethane). ¹H NMR (400 MHZ, DMSO-d₆) δ 10.90 (s, 1H), 8.17 (d, J=8.7 Hz, 1H), 7.85 (d, J=7.8 Hz, 1H), 7.69 (d, J=11.3 Hz, 2H), 7.65-7.58 (m, 2H), 7.50 (dd, J=16.0, 8.4 Hz, 2H), 6.96 (d, J=9.0 Hz, 1H), 3.74 (d, J=11.8 Hz, 1H), 3.17 (d, J=11.8 Hz, 1H), 2.48 (s, 3H). HRMS m/z [M+H]⁺ for C₂₂H₁₆N₄O₃S calculated 417.1021, found 417.1025. Purity—97.12% at 254 nm.

7-Bromo-5-(3-(5-thioxo-4,5-dihydro-1,2,4-oxadiazol-3-yl)phenyl)-1,5-dihydro-2H-naphtho[1,2-b][1,4]diazepine-2,4(3H)-dione triethylamine salt (21d; MRS4631): Following the procedure described for the synthesis of 21a, compound 20d (18 mg, 0.041 mmol) gave 21d as a white/yellowish solid (12 mg, 50%; R_{f}=0.3, TLC eluent=15% methanol in dichloromethane; purified by RP-HPLC, linear gradient of CH₃CN-10 mM TEAA in H₂O 30/70→50/50 for 40 min, flow rate=5.0 mL/min, Rₜ=26.8 min). ¹H NMR (400 MHZ, DMSO-d₆) δ 11.01 (s, 1H), 8.36-8.28 (m, 1H), 8.16-8.08 (m, 1H), 7.86 (d, J=7.8 Hz, 1H), 7.79 (qd, J=7.1, 3.5 Hz, 2H), 7.67 (d, J=2.2 Hz, 1H), 7.57 (t, J=7.9 Hz, 1H), 7.39 (dd, J=7.9, 2.1 Hz, 1H), 7.32 (s, 1H), 3.83 (d, J=11.9 Hz, 1H), 3.17 (d, J=11.9 Hz, 1H). HRMS m/z [M+H]⁺ for C₂₁H₁₃N₄O₃SBr calculated 480.9970, found 480.9976. Purity—95.83% at 254 nm.

10-((Methylthio)methyl)-5-(3-(5-thioxo-4,5-dihydro-1,2,4-oxadiazol-3-yl)phenyl)-1,5-dihydro-2H-naphtho[1,2-b][1,4]diazepine-2,4(3H)-dione triethylamine salt (21f; MRS4676): Following the procedure described for the synthesis of 21a, compound 20f (7.0 mg, 0.017 mmol) gave 21f as a white/yellowish solid (2.07 mg, 22%, purified by RP-HPLC, linear gradient of CH₃CN-10 mM TEAA in H₂O 35/65→50/50 for 40 min, flow rate=5.0 mL/min, Rₜ=22.8 min). ¹H NMR (400 MHZ, DMSO-d₆) δ 10.82 (s, 1H), 8.11 (s, 1H), 7.89 (d, J=8.4 Hz, 1H), 7.83 (d, J=7.7 Hz, 1H), 7.68 (d, J=9.0 Hz, 1H), 7.64-7.51 (m, 2H), 7.42-7.32 (m, 1H), 6.98 (d, J=8.9 Hz, 1H), 3.90 (s, 2H), 3.76 (d, J=11.8 Hz, 1H), 3.16 (d, J=11.8 Hz, 1H), 2.01 (s, 2H). HRMS m/z [M+H]⁺ for C₂₃H₁₈N₄O₃S₂ calculated 463.0899, found 463.0901. Purity—95.73% at 254 nm.

7-Bromo-10-methyl-5-(3-(5-thioxo-4,5-dihydro-1,2,4-oxadiazol-3-yl)phenyl)-1,5-dihydro-2H-naphtho[1,2-b][1,4]diazepine-2,4(3H)-dione triethylamine salt (21e; MRS4670): Following the procedure described for the synthesis of 21a, mixture of compounds 21a, e, g and h (35 mg, 0.084 mmol) gave 21a, e, g and h as a white/yellowish solid (R_{f}=0.5, TLC eluent=15% methanol in dichloromethane and 0.1% acetic acid). The products were separated by RP-HPLC using linear gradient of CH₃CN-10 mM TEAA in H₂O 30/70→45/55 for 40 min, flow rate=5.0 mL/min. This reaction also gave triethylamine salt of 21a (2.26 mg, 6%, Rₜ=24.5 min). Data for 21e: 1.79 mg, 4.5%, Rₜ=33.6 min. ¹H NMR (400 MHZ, DMSO-d₆) δ 10.88 (s, 1H), 8.12 (s, 1H), 8.01 (d, J=8.5 Hz, 1H), 7.86 (d, J=7.8 Hz, 1H), 7.66 (d, J=2.0 Hz, 1H), 7.62 (dd, J=8.6, 1.5 Hz, 1H), 7.56 (t, J=7.9 Hz, 1H), 7.37 (dd, J=7.9, 2.2 Hz, 1H), 7.23 (s, 1H), 6.54 (s, 1H), 3.80 (d, J=11.9 Hz, 1H), 3.17 (d, J=11.9 Hz, 1H), 2.58 (s, 3H). HRMS m/z [M+H]⁺ for C₂₂H₁₅N₄O₃SBr calculated 495.0126, found 495.0126. Purity—96.25% at 254 nm. Data for 10-(azidomethyl)-5-(3-(5-thioxo-4,5-dihydro-1,2,4-oxadiazol-3-yl)phenyl)-1,5-dihydro-2H-naphtho[1,2-b][1,4]diazepine-2,4(3H)-dione triethylamine salt (21g; MRS4633): 15 mg, 32%, Rₜ=26.3 min. ¹H NMR (400 MHZ, DMSO-d₆) δ 10.92 (s, 1H), 8.25 (s, 1H), 7.96 (d, J=8.3 Hz, 1H), 7.83 (d, J=7.7 Hz, 1H), 7.72 (d, J=9.0 Hz, 1H), 7.64 (d, J=1.9 Hz, 1H), 7.59 (dd, J=8.4, 1.6 Hz, 1H), 7.55 (t, J=7.9 Hz, 1H), 7.37 (dd, J=7.8, 2.2 Hz, 1H), 7.04 (d, J=9.0 Hz, 1H), 6.54 (s, 1H), 4.71 (s, 2H), 3.77 (d, J=11.8 Hz, 1H), 3.17 (d, J=11.8 Hz, 1H). MS m/z [M+H]⁺ for C₂₂H₁₅N₇O₃S calculated 458.1030, found 458.1. Purity—98.58% at 254 nm. Data for 10-(azidomethyl)-7-bromo-5-(3-(5-thioxo-4,5-dihydro-1,2,4-oxadiazol-3-yl)phenyl)-1,5-dihydro-2H-naphtho[1,2-b][1,4]diazepine-2,4(3H)-dione triethylamine salt (21h; MRS4634): 1.04 mg, 2%, Rₜ=35.7 min. ¹H NMR (400 MHZ, DMSO-d₆) δ 11.02 (s, 1H), 8.33 (s, 1H), 8.14 (d, J=8.6 Hz, 1H), 7.86 (d, J=7.8 Hz, 1H), 7.76 (d, J=8.6 Hz, 1H), 7.68 (s, 1H), 7.57 (t, J=7.8 Hz, 1H), 7.38 (d, J=8.0 Hz, 1H), 7.32 (s, 1H), 6.54 (s, 1H), 4.78 (s, 2H), 3.85 (d, J=12.0 Hz, 1H), 3.18 (d, J=11.9 Hz, 1H). MS m/z [M+H]⁺ for C₂₂H₁₄N₇O₃SBr calculated 536.0135, found 536.1. Purity—87.99% at 254 nm.

N-((2,4-Dioxo-5-(3-(5-thioxo-4,5-dihydro-1,2,4-oxadiazol-3-yl)phenyl)-2,3,4,5-tetrahydro-1H-naphtho[1,2-b][1,4]diazepin-10-yl)methyl) acetamide triethylamine salt (21i; MRS4673): Following the procedure described for the synthesis of 21a, compound 20i (25.0 mg, 0.058 mmol) gave 21i as a white/yellowish solid (7.07 mg, 19%, purified by RP-HPLC, linear gradient of CH₃CN-10 mM TEAA in H₂O 20/80→40/60 for 40 min, flow rate=5.0 mL/min, Rₜ=24.4 min). ¹H NMR (400 MHZ, DMSO-d₆) δ 10.90 (s, 1H), 8.45 (t, J=5.9 Hz, 1H), 8.13 (s, 1H), 7.88 (d, J=8.4 Hz, 1H), 7.82 (dt, J=7.8, 1.4 Hz, 1H), 7.68 (d, J=9.0 Hz, 1H), 7.60-7.48 (m, 3H), 7.38 (dd, J=8.1, 2.1 Hz, 1H), 6.98 (d, J=9.0 Hz, 1H), 6.54 (s, 1H), 4.57-4.35 (m, 2H), 3.74 (d, J=11.8 Hz, 1H), 3.16 (d, J=11.8 Hz, 1H), 1.92 (s, 3H). HRMS m/z [M+Na]⁺ for C₂₄H₁₉N₅O₄S calculated 496.1055, found 496.1062. Purity—95.62% at 254 nm.

9-(Azidomethyl)-5-(3-(5-thioxo-4,5-dihydro-1,2,4-oxadiazol-3-yl)phenyl)-1,5-dihydro-2H-naphtho[1,2-b][1,4]diazepine-2,4(3H)-dione triethylamine salt (21j; MRS4629): Following the procedure described for the synthesis of 21a, compound 20j,k (15.0 mg, 0.036 mmol) gave 21j,k as a white/yellowish solid (6.0 mg, 30%; R_{f}=0.2, TLC eluent=20% methanol in dichloromethane; purified by RP-HPLC, linear gradient of CH₃CN-10 mM TEAA in H₂O 30/70→50/50 for 40 min, flow rate=5.0 mL/min, Rₜ=26.8 min). ¹H NMR (400 MHZ, DMSO-d₆) δ 10.93 (s, 1H), 8.28 (d, J=8.8 Hz, 1H), 7.92 (d, J=1.7 Hz, 1H), 7.83 (d, J=7.8 Hz, 1H), 7.73 (d, J=9.0 Hz, 1H), 7.70-7.60 (m, 1H), 7.55 (t, J=7.9 Hz, 1H), 7.37 (dd, J=7.9, 2.2 Hz, 1H), 7.05 (d, J=9.0 Hz, 1H), 6.54 (s, 1H), 4.65 (s, 2H), 3.77 (d, J=11.8 Hz, 1H), 3.17 (d, J=11.8 Hz, 1H). HRMS m/z [M+H]⁺ for C₂₂H₁₅N₇O₃S calculated 458.1035, found 458.1034. Purity—94.85% at 254 nm. Data for 9-(azidomethyl)-7-bromo-5-(3-(5-thioxo-4,5-dihydro-1,2,4-oxadiazol-3-yl)phenyl)-1,5-dihydro-2H-naphtho[1,2-b][1,4]diazepine-2,4(3H)-dione triethylamine salt (21k; MRS4630): 2.0 mg, 9%; R_{f}=0.2, TLC eluent=20% methanol in dichloromethane; purified by RP-HPLC, linear gradient of CH₃CN-10 mM TEAA in H₂O 30/70→50/50 for 40 min, flow rate=5.0 mL/min, Rₜ=33.4 min. ¹H NMR (400 MHZ, DMSO-d₆) δ 11.02 (s, 1H), 8.35 (d, J=8.7 Hz, 1H), 8.10 (s, 1H), 7.86 (d, J=7.7 Hz, 1H), 7.77 (dd, J=8.8, 1.7 Hz, 1H), 7.69 (s, 1H), 7.57 (t, J=7.9 Hz, 1H), 7.41-7.36 (m, 1H), 7.35 (s, 1H), 6.54 (s, 1H), 4.76 (s, 2H), 3.84 (d, J=11.9 Hz, 1H), 3.18 (d, J=11.9 Hz, 1H). MS m/z [M+H]⁺ for C₂₂H₁₄N₇O₃SBr calculated 536.0135, 538.0115, found 536.0, 538.0. Purity—93.09% at 254 nm

7-Phenyl-5-(3-(5-thioxo-4,5-dihydro-1,2,4-oxadiazol-3-yl)phenyl)-1,5-dihydro-2H-naphtho[1,2-b][1,4]diazepine-2,4(3H)-dione triethylamine salt (211; MRS4672): Following the procedure described for the synthesis of 21a, compound 20l (17.0 mg, 0.039 mmol) gave 21l as a white/yellowish solid (18 mg, 80%, purified by RP-HPLC, linear gradient of CH₃CN-10 mM TEAA in H₂O 30/70→45/55 for 40 min, flow rate=5.0 mL/min, Rₜ=33.8 min). ¹H NMR (400 MHZ, DMSO-d₆) δ 11.01 (s, 1H), 8.34 (d, J=8.5 Hz, 1H), 7.85-7.65 (m, 3H), 7.59 (ddd, J=8.2, 6.9, 1.1 Hz, 1H), 7.53 (t, J=7.8 Hz, 1H), 7.49-7.36 (m, 4H), 7.34-7.25 (m, 2H), 6.87 (s, 1H), 3.85 (d, J=11.8 Hz, 1H), 3.21 (d, J=11.9 Hz, 1H). HRMS m/z [M+H]⁺ for C₂₇H₁₈N₄O₃S calculated 479.1178, found 479.1172. Purity—98.73% at 254 nm.

7-(Phenylethynyl)-5-(3-(5-thioxo-4,5-dihydro-1,2,4-oxadiazol-3-yl)phenyl)-1,5-dihydro-2H-naphtho[1,2-b][1,4]diazepine-2,4(3H)-dione (21m; MRS 4703): Following the procedure described for the synthesis of 21a, compound 20m (17 mg, 0.037 mmol) gave 21m as a white/yellowish solid (10 mg, 45%, R_{f}=0.30, TLC eluent=10% methanol in dichloromethane). ¹H NMR (400 MHZ, DMSO-d₆) δ 11.08 (s, 1H), 8.43-8.28 (m, 2H), 7.86 (d, J=7.8 Hz, 1H), 7.83-7.74 (m, 2H), 7.70 (d, J=2.1 Hz, 1H), 7.65 (dd, J=6.6, 3.0 Hz, 1H), 7.57 (t, J=7.9 Hz, 1H), 7.46-7.37 (m, 3H), 7.27 (s, 1H), 6.54 (s, 1H), 3.84 (d, J=11.9 Hz, 1H), 3.20 (d, J=11.9 Hz, 1H). HRMS m/z [M+H]⁺ for C₂₉H₁₈N₄O₃S calculated 503.1178, found 503.1170. Purity—95.85% at 254 nm.

7-(Pyridin-3-ylethynyl)-5-(3-(5-thioxo-4,5-dihydro-1,2,4-oxadiazol-3-yl)phenyl)-1,5-dihydro-2H-naphtho[1,2-b][1,4]diazepine-2,4(3H)-dione (21n; MRS4704): Following the procedure described for the synthesis of 21a, compound 20n (15 mg, 0.033 mmol) gave 21n as a white/yellowish solid (9 mg, 40%, R_{f}=0.15, TLC eluent=10% methanol in dichloromethane). ¹H NMR (400 MHZ, DMSO-d₆) δ 11.11 (s, 1H), 8.85 (d, J=2.2 Hz, 1H), 8.58 (dd, J=4.9, 1.7 Hz, 1H), 8.38 (ddd, J=17.8, 6.7, 4.1 Hz, 2H), 8.07 (dt, J=7.9, 2.0 Hz, 1H), 7.86 (d, J=7.8 Hz, 1H), 7.80 (qd, J=7.0, 3.6 Hz, 2H), 7.69 (d, J=2.4 Hz, OH), 7.58 (t, J=7.9 Hz, 1H), 7.48-7.37 (m, 1H), 7.32 (s, 2H), 6.54 (s, 1H), 3.85 (d, J=11.9 Hz, 1H), 3.20 (d, J=11.9 Hz, 1H). HRMS m/z [M+H]⁺ for C₂₉H₁₈N₄O₃S calculated 504.1130, found 504.1137. Purity—97.98% at 254 nm.

7-(Hex-1-yn-1-yl)-5-(3-(5-thioxo-4,5-dihydro-1,2,4-oxadiazol-3-yl)phenyl)-1,5-dihydro-2H-naphtho[1,2-b][1,4]diazepine-2,4(3H)-dione (21o; MRS4716): Following the procedure described for the synthesis of 21a, compound 20o (12 mg, 0.027 mmol) gave 21o as a white/yellowish solid (6 mg, 46%, R_{f}=0.10, TLC eluent=10% methanol in dichloromethane). ¹H NMR (400 MHZ, DMSO-d₆) δ 10.97 (s, 1H), 8.34-8.24 (m, 1H), 8.24-8.15 (m, 1H), 7.85 (d, J=7.8 Hz, 1H), 7.73 (qd, J=7.5, 3.6 Hz, 2H), 7.66 (t, J=1.9 Hz, 1H), 7.56 (t, J=7.9 Hz, 1H), 7.37 (d, J=8.1 Hz, 1H), 7.05 (s, 1H), 5.94 (s, 1H), 3.79 (d, J=11.9 Hz, 1H), 3.17 (d, J=11.8 Hz, 1H), 1.60-1.48 (m, 2H), 1.48-1.35 (m, 2H), 0.88 (t, J=7.4 Hz, 3H). HRMS m/z [M+H]⁺ for C₂₇H₂₂N₄O₃S calculated 483.1491, found 483.1496. Purity—98.47% at 254 nm.

7-Ethyl-5-(3-(5-thioxo-4,5-dihydro-1,2,4-oxadiazol-3-yl)phenyl)-1,5-dihydro-2H-naphtho[1,2-b][1,4]diazepine-2,4(3H)-dione triethylamine salt (21p; MRS4675): Following the procedure described for the synthesis of 21a, compound 20p (10.0 mg, 0.026 mmol) gave 21p as a white/yellowish solid (6.5 mg, 47%, purified by RP-HPLC, linear gradient of CH₃CN-10 mM TEAA in H₂O 30/70→50/50 for 40 min, flow rate=5.0 mL/min, Rₜ=27.7 min). 1H NMR (400 MHZ, DMSO-d₆) § 10.83 (s, 1H), 8.25 (d, J=8.2 Hz, 1H), 8.06 (d, J=8.0 Hz, 1H), 7.83 (d, J=7.7 Hz, 1H), 7.73-7.58 (m, 3H), 7.54 (t, J=7.9 Hz, 1H), 7.37 (dd, J=7.9, 2.2 Hz, 1H), 6.85 (s, 1H), 3.74 (d, J=11.7 Hz, 1H), 3.15 (d, J=11.8 Hz, 1H), 2.91 (ddq, J=22.5, 14.9, 7.4 Hz, 2H), 1.12-1.06 (m, 3H). HRMS m/z [M+H]⁺ for C₂₃H₁₈N₄O₃S calculated 431.1178, found 431,1178. Purity—98.48% at 254 nm.

10-(Aminomethyl)-5-(3-(5-thioxo-4,5-dihydro-1,2,4-oxadiazol-3-yl)phenyl)-1,5-dihydro-2H-naphtho[1,2-b][1,4]diazepine-2,4(3H)-dione triethylamine salt (21q; MRS4671): The azido compound 21g (15 mg, 0.027 mmol) was dissolved in THF-H₂O (9:1) and was added trimethylphosphine (PMe₃, 7.0 μL, 0.068 mmol). After stirring the reaction mixture for 5 h at room temperature, solvent was rotary evaporated and the residue was purified by RP-HPLC to afford 21q as light-yellow solid (11.7 mg, 82%, R_{f}=0.1, TLC eluent=20% methanol in dichloromethane; purified by RP-HPLC, linear gradient of CH₃CN-10 mM TEAA in H₂O 20/80→30/70 for 40 min, flow rate=5.0 mL/min, Rₜ=23.2 min). ¹H NMR (400 MHZ, DMSO-d₆) δ 8.28 (s, 1H), 7.92 (d, J=8.4 Hz, 1H), 7.83 (d, J=7.8 Hz, 1H), 7.70 (d, J=9.0 Hz, 1H), 7.63 (d, J=8.4 Hz, 1H), 7.59-7.51 (m, 2H), 7.42 (d, J=8.1 Hz, 1H), 7.01 (d, J=9.0 Hz, 1H), 4.09 (d, J=2.8 Hz, 2H), 3.74 (d, J=11.8 Hz, 1H), 3.19 (d, J=11.8 Hz, 1H). HRMS m/z [M+Na]⁺ for C₂₂H₁₇N₅O₃S calculated 454.0950, found 454.0950. Purity—97.36% at 254 nm.

5-(3-Methyl-5-(5-thioxo-4,5-dihydro-1,2,4-oxadiazol-3-yl)phenyl)-1,5-dihydro-2H-naphtho[1,2-b][1,4]diazepine-2,4(3H)-dione (21r; MRS4708): Following the procedure described for the synthesis of 21a, compound 20r (34 mg, 0.091 mmol) gave 21r as a white solid (20 mg, 53%, R_{f}=0.3, TLC eluent=20% methanol in dichloromethane). ¹H NMR (400 MHz, DMSO-d₆) δ 10.95 (s, 1H), 8.28 (d, J=8.5 Hz, 1H), 7.93 (d, J=8.0 Hz, 1H), 7.76-7.65 (m, 3H), 7.61 (t, J=7.5 Hz, 1H), 7.54 (s, 1H), 7.37 (s, 1H), 7.02 (d, J=9.0 Hz, 1H), 3.76 (d, J=11.8 Hz, 1H), 3.18 (dd, J=11.8, 1.6 Hz, 1H), 2.40 (s, 3H). HRMS m/z [M+H]⁺ for C₂₂H₁₇N₄O₃S calculated 417.1021, found 417.1029. Purity—99.82% at 254 nm.

5-(4-(5-Thioxo-4,5-dihydro-1,2,4-oxadiazol-3-yl) pyridin-2-yl)-1,5-dihydro-2H-naphtho[1,2-b][1,4]diazepine-2,4(3H)-dione triethylamine salt (21s; MRS4717): Following the procedure described for the synthesis of 21a, compound 20s (25 mg, 0.069 mmol) gave 21s as a white solid (10 mg, 29%, R_{f}=0.3, TLC eluent=20% methanol in dichloromethane; purified by RP-HPLC, linear gradient of CH₃CN-10 mM TEAA in H₂O 10/90→45/55 for 40 min, flow rate=5.0 mL/min, Rₜ=28.2 min). ¹H NMR (400 MHZ, DMSO-d₆) δ 10.93 (s, 1H), 8.49 (d, J=5.1 Hz, 1H), 8.25 (d, J=8.5 Hz, 1H), 8.13 (s, 1H), 7.93 (d, J=8.0 Hz, 1H), 7.77 (d, J=5.1 Hz, 1H), 7.68 (dd, J=12.8, 8.5 Hz, 2H), 7.60 (t, J=7.5 Hz, 1H), 6.96 (d, J=8.9 Hz, 1H), 3.80 (d, J=11.8 Hz, 1H), 3.19 (d, J=11.8 Hz, 1H). HRMS m/z [M+H]⁺ for C₂₀H₁₃N₅O₃S calculated 404.0817, found 404.0816. Purity—97.61% at 254 nm.

5-(5-(5-Thioxo-4,5-dihydro-1,2,4-oxadiazol-3-yl) pyridin-3-yl)-1,5-dihydro-2H-naphtho[1,2-b][1,4]diazepine-2,4(3H)-dione triethylamine salt (21t; MRS4718): Following the procedure described for the synthesis of 21a, compound 20t (25 mg, 0.069 mmol) gave 21t as a white solid (19 mg, 54%, R_{f}=0.3, TLC eluent=10% methanol in dichloromethane; purified by RP-HPLC, linear gradient of CH₃CN-10 mM TEAA in H₂O 10/90→45/55 for 40 min, flow rate=5.0 mL/min, Rₜ=32.3 min). ¹H NMR (400 MHZ, DMSO-d₆) δ 10.93 (s, 1H), 8.96 (d, J=1.9 Hz, OH), 8.56 (d, J=2.5 Hz, 1H), 8.28 (d, J=8.5 Hz, 1H), 8.02 (t, J=2.2 Hz, 1H), 7.95 (d, J=7.9 Hz, 1H), 7.74 (d, J=9.0 Hz, 1H), 7.69 (dd, J=8.6, 6.4 Hz, 1H), 7.62 (t, J=7.4 Hz, 1H), 7.06 (d, J=9.0 Hz, 1H), 3.79 (d, J=11.9 Hz, 1H), 3.21 (d, J=11.9 Hz, 1H). HRMS m/z [M+H]⁺ for C₂₀H₁₃N₅O₃S calculated 404.0817, found 404.0816. Purity—99.21% at 254 nm.

5-(2-(5-Thioxo-4,5-dihydro-1,2,4-oxadiazol-3-yl) pyridin-4-yl)-1,5-dihydro-2H-naphtho[1,2-b][1,4]diazepine-2,4(3H)-dione triethylamine salt (21u; MRS 4719): Following the procedure described for the synthesis of 21a, compound 20u (25 mg, 0.069 mmol) gave 21u as a white solid (20 mg, 57%, R_{f}=0.3, TLC eluent=10% methanol in dichloromethane; purified by RP-HPLC, linear gradient of CH₃CN-10 mM TEAA in H₂O 20/80→40/60 for 40 min, flow rate=5.0 mL/min, Rₜ=32.3 min). ¹H NMR (400 MHZ, DMSO-d₆) δ 10.95 (s, 1H), 8.92 (s, 1H), 8.69 (d, J=5.3 Hz, 1H), 8.28 (d, J=8.4 Hz, 1H), 7.97 (d, J=8.0 Hz, 1H), 7.76 (d, J=9.0 Hz, 1H), 7.73-7.60 (m, 2H), 7.41 (dd, J=5.3, 2.2 Hz, 1H), 7.06 (d, J=8.9 Hz, 1H), 6.52 (s, 1H), 3.82 (d, J=11.9 Hz, 1H), 3.19 (d, J=11.9 Hz, 1H). HRMS m/z [M+H]⁺ for C₂₀H₁₃N₅O₃S calculated 404.0817, found 404.0818. Purity—99.20% at 254 nm.

5-(6-(5-Thioxo-4,5-dihydro-1,2,4-oxadiazol-3-yl) pyridin-2-yl)-1,5-dihydro-2H-naphtho[1,2-b][1,4]diazepine-2,4(3H)-dione triethylamine salt (21v; MRS4720): Following the procedure described for the synthesis of 21a, compound 20v (25 mg, 0.069 mmol) gave 21v as a white solid (19 mg, 54%, R_{f}=0.3, TLC eluent=10% methanol in dichloromethane; purified by RP-HPLC, linear gradient of CH₃CN-10 mM TEAA in H₂O 20/80→40/60 for 40 min, flow rate=5.0 mL/min, Rₜ=35.9 min). ¹H NMR (400 MHZ, DMSO-d₆) δ 10.94 (s, 1H), 8.24 (d, J=8.4 Hz, 1H), 8.12 (t, J=7.8 Hz, 1H), 7.92 (d, J=7.7 Hz, 2H), 7.76-7.64 (m, 3H), 7.60 (t, J=7.4 Hz, 1H), 6.95 (d, J=9.0 Hz, 1H), 6.52 (s, 1H), 3.78 (d, J=11.8 Hz, 1H), 3.22-3.13 (m, 1H). HRMS m/z [M+H]⁺ for C₂₀H₁₃N₅O₃S calculated 404.0817, found 404.0818. Purity—98.90% at 254 nm.

5-(3-(5-Thioxo-4,5-dihydro-1,2,4-oxadiazol-3-yl)phenyl)-1,5-dihydro-2H-naphtho[1,2-b][1,4]diazepine-2,4(3H)-dione sodium salt (NP-1815-PX—Na salt; 22a): Compound 21a (10.0 mg, 25 μmol) was suspended in 1:1 acetonitrile-water and was added 0.1M NaOH solution (250 μL, the solution turned clear after shaking, particles/turbidity if any, was filtered off using Nylon Acrodisc® filters). The clear solution was frozen and lyophilized to get 22a as pale-yellow solid (10.7 mg, quantitative yield). ¹H NMR (400 MHZ, Deuterium Oxide) δ 8.10 (d, J=8.5 Hz, 1H), 7.75-7.58 (m, 4H), 7.48 (dt, J=18.4, 7.7 Hz, 2H), 7.39 (d, J=9.1 Hz, 1H), 7.28 (d, J=8.0 Hz, 1H), 6.82 (d, J=9.0 Hz, 1H), 3.67 (d, J=12.2 Hz, 1H), 3.36 (d, J=11.9 Hz, 1H). 22b and 22c were prepared in same way and used as such without further analysis. Both sodium and triethylamine salts were water-soluble.

### Biological Assays

The antagonism at hP2XRs by various synthesized analogues of 5 using a fluorescent Ca²⁺ sensitive dye (Fluo-8 NW) or a luminescence Ca²⁺ sensitive photoprotein as readout, using a FLIPR^{TETRA} to measure the emitted light. The assays of the test compounds were performed in a 384 format. The assays hP2RX2/P2X3 and hP2RX4 used a Ca²⁺ sensitive dye (Fluo-8 NW no wash calcium assay kit, AAT Bioquest, Sunnyvale, California, cat. #36316) with fluorescent measurement, and the hP2RX1 and hP2RX3 assays used coelenterazine (preloaded in the cells during an incubation for 3 h at 37° C., Biosynth AG, Staad, Switzerland, cat. #C-7001) and a Ca²⁺-responsive photoprotein with a signal of luminescence. For hP2RX1 and hP2RX3 assays, the cells were seeded at 10,000 cells/well in 384 well plates in complete medium (25 μL/well). Following the removal of medium and pre-loading with coelenterazine in fresh medium (10 μM, 30 μL per well), the test compound was added to each well (10 μL per well) and the emitted light recorded. After 30 min incubation, the reference agonist (at ˜EC₈₀) was added, and the luminescence again recorded. For hP2RX2/P2X3 and hP2RX4 assays, the cells were seeded at 10,000 cells/well in 384 well plates in complete medium (25 μL/well). After 24 h the medium was removed and replaced with calcium sensitive dye (Fluo-8 NW, 20 μL/well) in assay buffer. After a 1 h incubation at room temperature in the dark, the test compound was added to each well (10 μL per well) and the fluorescence recorded. After 30 min, the reference agonist (at ˜EC₈₀) was added, and the fluorescence again recorded.

The following standard P2XR ligands were used (prepared at 10 mM in water and stored in aliquots at −20° C.): agonist α,β-Me-ATP: (Sigma-Aldrich, cat. #M6517); P2X4 agonist CTP (Sigma-Aldrich, cat. #C1506); antagonist TNP-ATP: (Tocris Bio-Techne SRL, Milan, Italy, cat. #2464). The assay buffer consisted of Standard Tyrode's Buffer: (in house solution), 130 mM NaCl, 5 mM KCl, 2 mM CaCl₂, 5 mM NaHCO₃, 1 mM MgCl₂, 20 mM HEPES, pH 7.4. For hP2X1, hP2RX2/P2X3 and hP2X3, α,β-Me-ATP was used at 100, 31.6, 10, 3.16, 1, 0.316, 0.1 and 0.0316 μM. For hP2X4, CTP was used at 100, 31.6, 10, 3.16, 1, 0.316, 0.1 and 0.0316 μM. The reference agonist (at ˜EC₈₀) was 1 μM α,β-Me-ATP for hP2X1 and hP2X3, 1 μM α,β-Me-ATP for hP2X/P2X3 and 10 μM CTP for hP2X4.

### Results of Compound Testing Biological Assay

The compounds as shown in Table 4 were tested for inhibition of human (h) P2XRs, including at homotrimeric P2X1R, P2X3R, and P2X4R and at heteromeric P2X2/3R. The assays used HEK-293 cells stably transfected with P2RX4R and CHO-K1 cells stably transfected with either P2X1R, P2X2/3R, and P2X3R in 96-well and 386-well plates. Detection of each well was by luminescence or fluorescence using an imaging plate reader. Full dose response curves were determined for both reference agonist (TNP-ATP) and a known antagonist (Compound 22a) at each receptor. The IC₅₀ values (μM) reported for the test compounds represent the inhibition of an approximately EC₈₀ concentration of the reference agonist. A robust Z′ (RZ′) parameter was determined for each assay plate and ranged from 0.683 to 0.903, which indicated the robust quality of the assay. The antagonists were not evaluated at the mouse (m) P2X4R.

TABLE 4
P2X channel assay results.^{a}

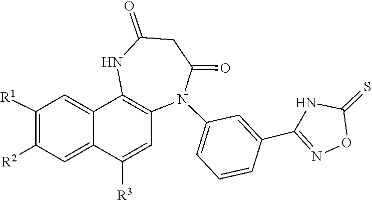

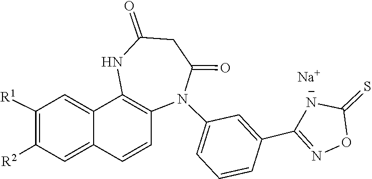

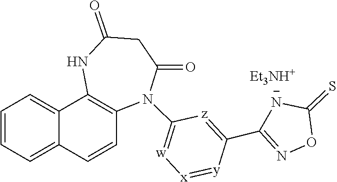

| | | hP2X1R | hP2X2/3R | hP2X3R | hP2X4R |
|---|---|---|---|---|---|
| | | (IC₅₀ or % | (IC₅₀ or % | (IC₅₀ or % | (IC₅₀ or % |
| Compound | Structure | inhib.)^{b} | inhib.)^{b} | inhib.)^{b} | inhib.)^{b} |
| TNP-ATP | — | 0.120 | 0.064 | 0.018 | 2.77 |
| 22a^{c} | R¹ = R² = R³ = H | −6% | 10% | 3% | 1.35 |
| 22b | R¹ = CH₃, R² = R³ = H | 6% | 9% | 13% | 17.9 |
| 22c^{d} | R¹ = H, R² = CH₃, R³ = H | −4% | 11% | 6% | 1.38 |
| 21d^{d} | R¹ = R² = H, R³ = Br | 5% | 11% | 7% | 9.51 |
| 21e | R¹ = CH₃, R² = H, R³ = Br | −2% | −5% | 17% | 11.8 |
| 21f | R¹ = CH₂SCH₃, R² = R³ = H | ND | ND | ND | ND |
| 21g | R¹ = CH₂N₃, R² = R³ = H | 8% | 4% | 13% | 6% |
| 21h | R¹ = CH₂N₃, R² = H, R³ = Br | −13% | −4% | 15% | 8% |
| 21i | R¹ = CH₂NHAc, R² = R³ = H | 26% | −5% | 9% | 16% |
| 21j | R¹ = H, R² = CH₂N₃, R³ = H | −5% | 2% | 0% | 21.3 |
| 21k | R¹ = H, R² = CH₂N₃, R³ = Br | ND | ND | ND | ND |
| 211 | R¹ = R² = H, R³ = Ph | 14% | 10% | 11% | 20.7 |
| 21m | R¹ = R² = H, R³ = C≡C—Ph | 14% | 5% | 7% | −4% |
| 21n | R¹ = R² = H, R³ = C≡C—(3-Py) | 9% | 13% | 9% | 34% |
| 21o | R¹ = R² = H, R³ = C≡C—n-Bu | 16% | 1% | 18% | 15% |
| 21p | R¹ = R² = H, R³ = Et | 6% | 10% | 6% | 5.13 |
| 21q | R¹ = CH2NH2, R² = R³ = H | 8% | 0% | 9% | 9% |
| 21r | X = CCH3, W = Y = Z = CH | 16% | 5% | 12% | 14.0 |
| 21s | W = N, X = Y = Z = CH | 18% | −6% | 18% | 13.5 |
| 21t | X= N, W = Y = Z = CH | 15% | 25% | 6% | 14.6 |
| 21u^{d} | Y=N, W = X =Z = CH | 15% | 0% | 11% | 0.503 |
| 21v | Z = N, W = X = Y = CH | 31% | 15% | 17% | 6.48 |

ªMeasured at hP2X4R (fluorescence read-out assay), at hP2X1R and hP2X3R (luminescence), and at hP2X2/3R (fluorescence). Fixed agonist concentration roughly corresponds to the predetermined EC₈₀ value of each: CTP, 10 μM at P2X4R; α,β-MeATP: 1.0 μM at P2X1R, 5.0 μM at P2X2/3R, 1.0 μM at P2X3R.
^{b}IC₅₀ in μM, or percent inhibition at 30 μM concentration in italics. Each data point was determined in quadruplicate.
^{c}Compound 5.
^{d}Compound correspondence: 21c, MRS 4596; 21d, MRS 4631; 21u, MRS 4719.

The antagonists were not evaluated at the mouse (m) P2X4R, but the comparative (lead) compound 5 (compound 22a) was as potent in blocking mP2X4R effects in vitro as at hP2X4R and also showed considerable in vivo efficacy in the mouse.

The 6-methyl substitution of the naphthalene core (22c) did not lower the inhibitory activity of known antagonist 22a, However, a 7-methyl substitution alone in 22b decreased activity by 13-fold, and larger/polar substitution with azidomethyl 22h or acetamidomethyl 22i at this position rendered the molecule inactive. A large azidomethyl group at the 6-position (21j) was destabilizing, although not to the same extent as 7-substituted analogues. However, a 4-bromo substitution in the absence (21d) or presence of 6-methyl (21e) only moderately reduced activity.

Off-target binding activity of 5, 21c, 21d and 21u was determined at 45 receptors, channels and transporters by the NIMH Psychoactive Drug Screening program (Besnard, J.; Ruda, G. F.; Setola, V.; Abecassis, K.; Rodriguiz, R. M.; Huang, X. P.; Norval, S.; Sassano, M. F.; Shin, A. I.; Webster, L. A.; Simeons, F. R.; Stojanovski, L.; Prat, A.; Seidah, N. G.; Constam, D. B.; Bickerton, G. R.; Read, K. D.; Wetsel, W. C.; Gilbert, I. H.; Roth, B. L.; Hopkins, A. L. Automated design of ligands to polypharmacological profiles. Nature 2012, 492, 215-220). Significant binding of 21u (MRS 4719)(mean±SD inhibition at 10 μM) occurred only at D₃ dopamine (74±10%) and H₂ histamine (89±4%) receptors. There were no significant interactions at 10 μM for 5, 21c and 21d.

### In Vivo Experiments

### 1.1 Experimental Design and Animals

Age- and weight-matched, equal numbers of male and female C57B/6 (wild-type) WT mice from a breeding colony at UConn Health animal facility were used. Mice were fed standard chow diet and water ad libitum. Standard housing conditions were maintained at a controlled temperature with a 12-h light/dark cycle. All experiments were approved by the Institutional Animal Care and Use Committee of University of Connecticut Health and conducted in accordance with the U.S. National Institutes of Health Guidelines for the Care and Use of Laboratory Animals.

A total of 34 (eight- to twelve-week-old) mice were randomly divided into vehicle (alzet minipump containing 1×PBS), MRS 4719 (Alzet minipump containing 0.5-3 mg/kg/day for 3 days) and MRS-4596 (Alzet minipump containing 5.0 mg/kg/day for 3 days) groups and subjected Alzet minipump implantation immediately after initiation of reperfusion. Alzet minipump starts releasing experimental compounds within 3-4 hours after implantation. After 3 days, all the mice were sacrificed to isolate brain and kept in deep freezer until further use. Three mice were excluded from the study due to death during reperfusion (2 Veh and 1 MRS 4596 treatment)

### 1.2 Middle Cerebral Artery Occlusion (MCAo)

Transient focal cerebral ischemia was induced by a 60 min right MCAo under isoflurane anesthesia followed by reperfusion for either 3 days as described previously (Verma R., et. al. 2017 doi: 10.1016/j.bbi.2017.07.155). Briefly, we proceeded with midline ventral neck incision and unilateral right MCAo using a 6.0 silicone rubber-coated monofilament (size 602145/602245; Doccol Corporation, Sharon, MA) placed 10-11 mm away from the bifurcation point of the internal carotid artery through an external carotid artery stump. Rectal temperatures were monitored and maintained at 37±0.5° C. with the help of a heating pad. We used laser Doppler flowmetry to measure cerebral blood flow and to confirm occlusion and reperfusion. All animals were fed wet mash for one week after surgery to ensure adequate nutrition for chronic endpoints, as animals have rearing deficits after stroke.

### 1.3 Measurement of the Cerebral Infarct Volume

The isolated brains were cut into 5 equal coronal sections and stained with TTC (1.5% solution in PBS) for 20 mins and then fixed in 10% buffered formaldehyde solution. The stained brain slices were digitally photographed and the infarct area, of each brain was measured in a blinded manner, using an image analysis software, Sigmascan Pro 5. The infarct volume was calculated by Swanson's method (Swanson et al., 1990) to correct for edema. The total volumes of both contralateral and ipsilateral hemisphere, and the volumes of the striatum, cortex in both hemispheres were measured and the infarct percentage was calculated as % contralateral structure to avoid mis-measurement secondary to edema.

1.4 Statistics: Data In Vivo Experiment were Represented as Mean±SD.

Significance was determined using Student's t-test (for two groups) or one-way analysis of variance (ANOVA) (for more the two groups followed by Tukey' post hoc test) for comparing the experimental groups between vehicle and drug treatment after stroke (GraphPad Prism Software Inc., San Diego, CA)

### In Vivo Experimental Results

The results of the aza-scan showed the substantial effect of a ring nitrogen, either to enhance (4-pyridyl, 21u) or to reduce (21s, 21t, 21v) inhibitory activity with respect to reference antagonist 22a. The active compounds were selective for the P2X4R; the most inhibition observed at any other receptor was at the P2X1R, which was ≤31% at 30 μM for compounds 21v and 21i.

Based on IC₅₀ values, the two most potent P2X4R antagonists were chosen for evaluation of in the in vivo activity assay. It was previously shown that commercially available P2X4R inhibitor 5-BDBD ([5-(3-bromophenyl)-1,3-dihydro-2H-benzofuro[3,2-e]-1,4-diazepin-2-one], compound 3) (1 mg/kg/day for 3 days) reduced stroke infarct size when administered at onset of acute stroke for a total of 3 days. Using the same pharmacological paradigm, MRS 4719 was infused at 0.5-3 mg/kg/day for 3 days.

### Neuroprotective Activity of MRS 4719 and MRS 4596

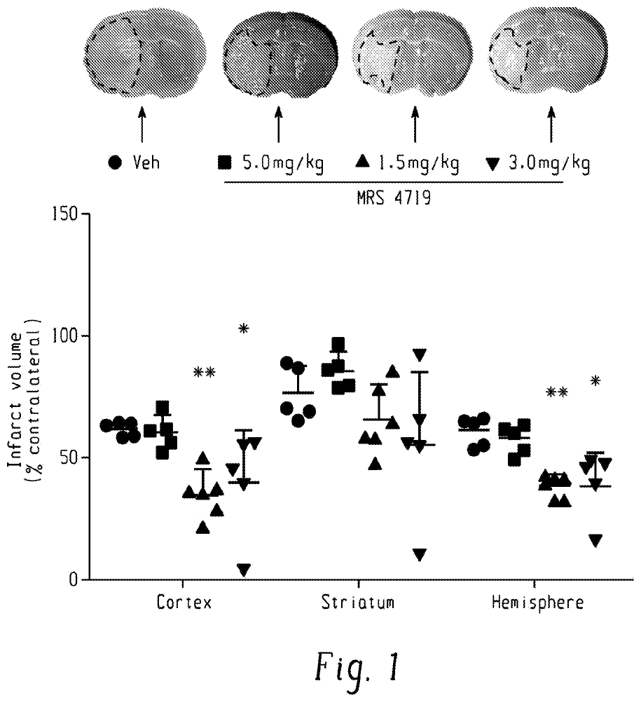

FIG. 1 shows the dose-dependent effect of MRS 4719 (0.5-3 mg/kg/day×3 days continuous infusion with alzet minipump) on infarct volume after 3 days of ischemic stroke. The upper panel of FIG. 1 shows representative TTC stained sections depicting infarct area (dotted line. The lower panel FIG. 1 shows quantification of infarct volume (% contralateral) in various regions of brain. MRS 4719 treatment significantly reduced total hemispheric and cortical infarct volume (n=5-6/dose). **p<0.01; vs. Veh *p<0.05; vs. Veh in respective brain regions (One-way ANOVA followed Tuckey's post hoc test). Data are presented as Mean±S.D.

Thus, it was found that both doses 1.5 and 3.0 mg/kg caused significant neuroprotection based on total hemispheric infarct volume size as the outcome. These data showed that ceiling effect was observed at a dose of 1.5 mg/kg, which showed greater level of reduction in both cortical and total (hemispheric) infarct volume as compared to high dose (FIG. 1). There was no statistically significant decrease in the striatal infarct volume after MRS 4719 treatment. Notably, the striatal area of the brain is often refractory to treatment due to it being a core area in stroke damage.

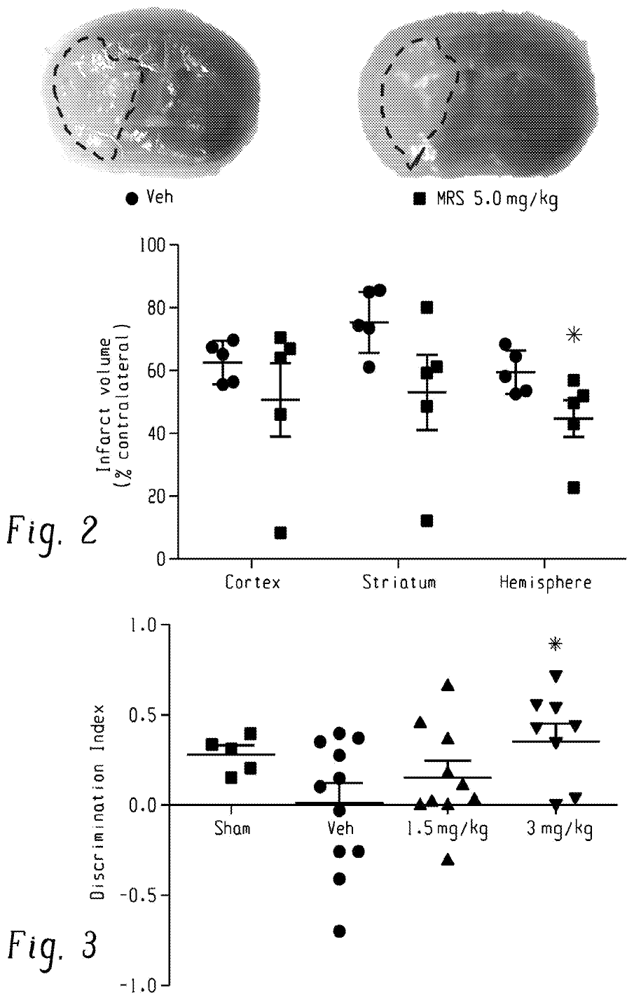

In studying the less potent MRS 4596, which has a higher IC50 value at inhibiting P2X4 receptors, the higher dose of 5 mg/kg/day was tested. FIG. 2 shows the effect of MRS 4596 post-treatment (5.0 mg/kg/day for 3 days continuous infusion with alzet minipump) on infarct volume at 3 days after stroke. A representative TTC stained section showing infarct area (dotted line) is shown the in upper portion of FIG. B and graph showing quantification of infarct volume (% contralateral) is shown in the lower panel of FIG. B. For these data, (*p<0.05; vs Veh) reduced total hemispheric infarct volume was observed (n=5-7/treatment group). Data are presented as Mean±S.D.

A moderate but significant decrease in total hemispheric infarct volume was found. The effects were not statistically significant on cortical or striatal infarct volume alone. These data are consistent with a prior finding that short-term blockage of P2X4R confers acute neuroprotection after stroke (Srivastava, P., Cronin, C. G., Scranton, V. L., Jacobson, K. A., Liang, B. T., Verma, R. Neuroprotective and neuro-rehabilitative effects of acute purinergic receptor P2X4 (P2X4R) blockade after ischemic stroke. Experimental Neurology, Exp. Neurol., 2020, 329:113308. https://doi.org/10.1016/j.expneurol.2020.113308).

### 1.4 Effect of MRS 4719 Treatment on Sensorimotor as Well as Learning and Memory Deficit.

To optimize optimum duration of treatment with MRS-4719 in middle aged mice, mice were subjected to sensorimotor and learning and memory task for 4 weeks after stroke at different time interval. Both Rota-rod and NORT respectively, were used.

Rotarod test: Motor coordination in rodents was examined by rotarod test. Mice were placed on a rotating cylindrical rod that accelerated from 4-40 rotations per min, for 5 min duration. There were two trials per subject including a 30 min break in between. The latency of falling from the rotating rod per trial (in sec) was recorded, and mean latency was used for comparing the two groups

NORT: The novel recognition task (NORT) is used to evaluate cognition, particularly recognition memory, in rodent models of CNS (central nervous system) disorders. This test measures the time spent by a mouse exploring a novel object compared a familiar one. This preference assesses intact recognition memory as detailed in Verma, et al. (Verma R, Friedler B D, Harris N M, Mccullough L D. Pair housing reverses post-stroke depressive behavior in mice. Behav Brain Res. 2014 Aug. 1; 269:155-63. doi: 10.1016/j.bbr.2014.04.044. Epub 2014 May 2. PMID: 24793492; PMCID: PMC4114771) Briefly, Mice were placed in the behavioral room for acclimatization for 1 hour. During habituation animals were allowed to explore an empty arena for at least 10 minutes. After habituation, animals were exposed to the familiar arena with 2 identical objects placed at an equal distance for 10 minutes (trial phase). If the total time of exploration of these objects was greater than 20 seconds, these mice qualified for the experimental test, which was conducted 24 hours after the trial. One of the objects from the trial was replaced with a novel object. Mice were then again allowed to explore the test arena for 10 minutes. The experiment was recorded and analyzed using Any maze software (Any maze software Inc.) by a trained observer. A discrimination index (DI) was calculated by using the formula DI=(TN−TF)/(TN+TF), where TN=time spent exploring the novel object and TF=time spent in exploring of familiar objects. The NORT was performed on days 28 after stroke and analyzed by an experimenter blinded to treatment.

Both MRS 4719- and Vehicle-treated, middle-aged, mice were assessed via NORT recognition task four weeks after MCAo. As illustrated in FIG. 3, the mice showed dose dependent improvement in learning and memory function after stroke and a dose of 3 mg/kg showed significant retention of memory (p<0.05 vs. Veh.) after ischemic stroke, suggesting neurorehabilitatory effects after short-term treatment with MRS 4719. (n=5-11/treatment group=32 mice total; 19 males and 13 females; data are Mean±S.D.)

In the Rotarod test mice were placed on a rotating cylindrical rod that accelerated from 4-40 rotations per min, for 5 min duration. There were two trials per subject including a 30 min break in between. The latency of falling from the rotating rod per trial (in sec) was recorded, and mean latency was used for comparing the between the groups. (PMID 36150180. Short term treatment for two days improved sensorimotor during progressive recovery as shown by increase in latency to fall time (*p<0.05 vs Veh, One-way anova followed by Tukey's multiple comparison test) measured by Novel Object Recognition Test. (n=5-15/treatment group=50 mice total; 28 males and 22 females). Data are Mean±standard deviation (S.D.)

A dose of 1.5 mg/kg for 3 days did not show any significant effect on memory and learning (0157), but reducing MRS 4719 treatment duration to 2 days in place of 3 or 7 days, significantly increased memory retention as measured by NORT. This data suggest that a short term rather than long term treatment is beneficial using P2X4R inhibition therapy, which is consistent with our previous observation (PMID 32289314 and 28751018).

### Myocardial Ischemia/Reperfusion Experiments

### 1. Methods

### 1.1 Myocardial Ischemia/Reperfusion

Ten-12 week old male and female mice (approx. 25 g bw) were anesthetized with Ketamine/Xylazine (100/5 mg/Kg) mix and were ventilated with air via a mouse ventilator prior surgery. A thoracotomy was performed at the third or the fourth intercostal space to visualize the epicardial coronary artery, LAD. The LAD was ligated for 30 or 60 minutes with an 7-0 microsurgery nylon suture (Ethilon) with a piece of polyethylene-10 tubing placed over the left coronary artery. Ischemia was confirmed by bleaching of the myocardium and ventricular tachyarrhythmia. After 30-min (for determining infarct size) or 60-min (for determining cardiac function) occlusion, the ligature is released and the polyethylene tubing is removed to initiate the reperfusion for predetermined time. A 48 hour reperfusion was used to quantify the effect of the drug/veh on infarct size). And a reperfusion for a duration of either two weeks or 4 weeks was used for determining of cardiac function by echocardiography. Sham (control) operation involved an identical procedure except that the suture was passed through the myocardium without being tied and the suture was then removed. The chest wall and skin were closed in two layers and the animals allowed to recover.

### 2.2 Drug Administration and Determination of Cardiac Function

Vehicle or MRS4719 (2 mg/kg/day; continuous infusion) was given subcutaneously using a micro-osmotic alzet minipump (designed to continuously release the drug up to 3 days) immediately after reopening of the occluded LAD.

For the group of mice in which effect of MRS 4719 on infarct size was studied, the vehicle and MRS4719 treated mice were euthanized at end of the 48 hours after occlusion. Freshly isolated hearts were used for infarct size determination. For the groups of mice in which effects on cardiac function were studied, MRS 4719 infusion with alzet minipump was given for a total 3 days after a 60 min LAD occlusion. In this group mice were reperfused either for 2 or 4 weeks. At end of reperfusion, echocardiography was performed to determine cardiac function and hearts were collected for further future analysis. Data were analyzed as changes in cardiac echocardiography function parameters at baseline before LAD ligation vs. same parameters after either 2 weeks or 4 weeks of reperfusion for each mouse. Statistical analysis on changes in the parameters was by unpaired t test.

### 2. Results

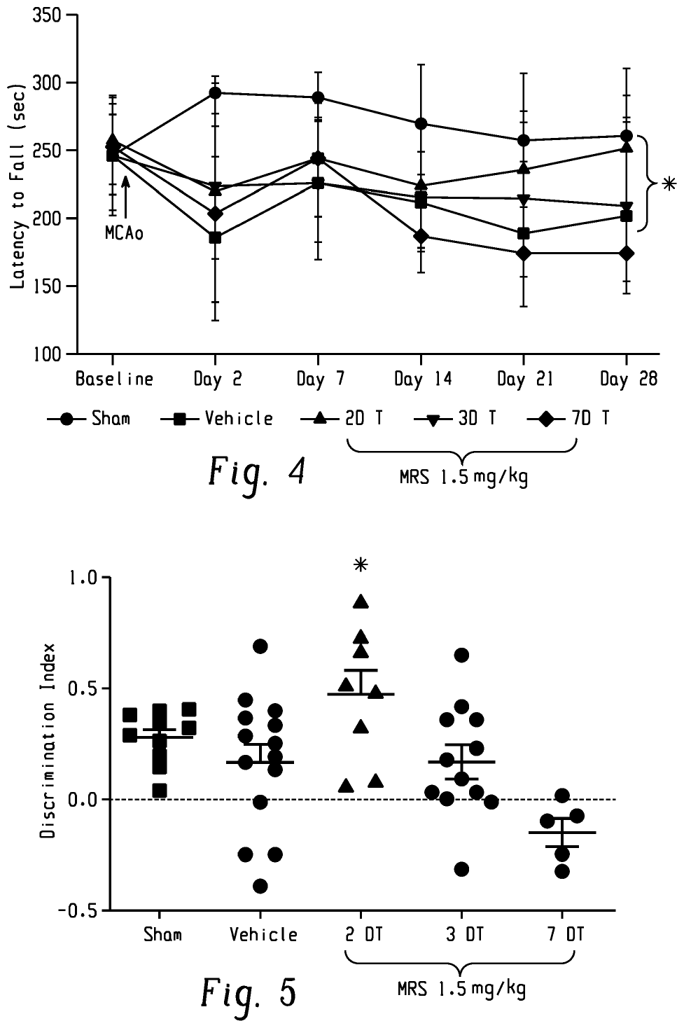

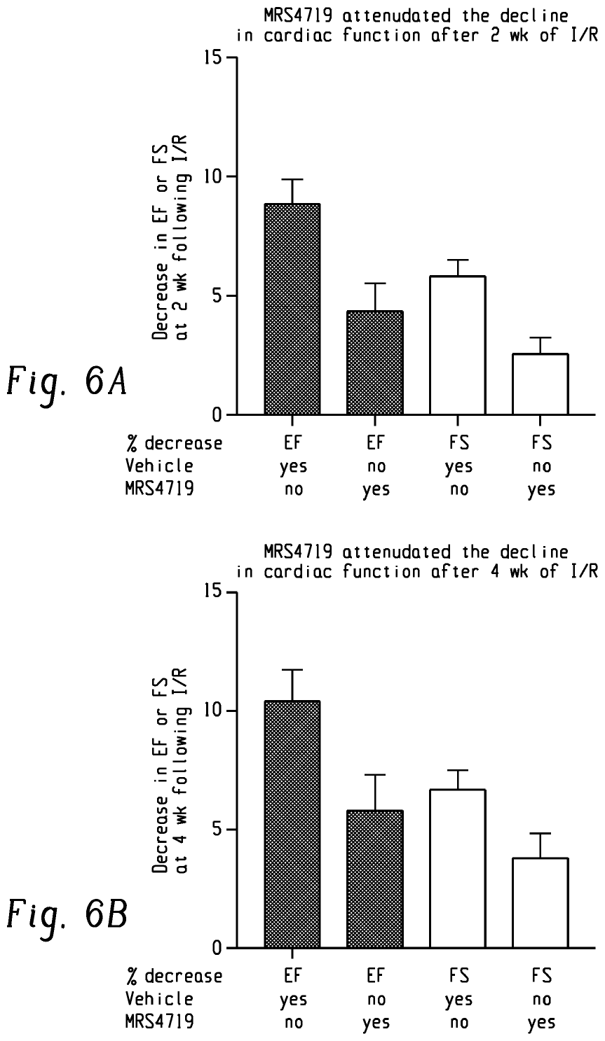

FIGS. 4A and 4B illustrate that acute or short term systemic infusion of MRS4719 at reperfusion blocked the subsequent development of cardiac dysfunction. In particular, at 2 weeks (FIG. 4A) or 4 weeks (FIG. 4B) following reperfusion, echocardiography-derived left ventricular ejection fraction (EF) and fractional shortening (FS) were obtained. MRS4719-treated animals showed a lesser degree of decline in EF and FS than vehicle-treated animals. Data were mean and SEM. Decreases in EF and FS in MRS4719 treated mice were less than those in vehicle-treated mice at 2 weeks or 4 weeks post-I/R (P<0.05, t test).

In summary, the inventors have synthesized analogues and analyzed the P2X4 receptor structure activity relationship of a series of substituted 1,5-dihydro-2H-naphtho[1,2-b][1,4]diazepine-2,4(3H)-diones. Extensive modification of the reported synthetic route was required for the desired aromatic functionalization and aza-scan. 4-Pyridyl compound 21u and 6-methyl compound 22c analogues were the most potent analogues (human (h) P2X4R IC₅₀ of 0.503 and 1.38 μM, respectively) and were selective versus hP2X1R, hP2X2/3R and hP2X3R. These compounds also showed neuroprotective activity when tested in the middle cerebral artery model of ischemic stroke in mice. The 4-pyridyl 21u analogue was found to be most potent. Thus, the 6 position, but not 7 position of the naphthalene ring is amenable to substitution, and an aza-scan of the N-phenyl ring indicated a strong preference for the 4-pyridyl group compared to other positions of N incorporation in the ring. In addition, systemic administration of MRS4719 at time of reperfusion improved subsequent cardiac function in an established murine model of cardiac ischemia/reperfusion I/R.

The following abbreviations (abbr.) are used herein.

| Abbr. | Term | Abbr. | Term |
|---|---|---|---|
| Bz | Benzyl | min | Minute(s) |
| DBU | 1,8- | NBS | N-bromosuccinimide |
| | Diazabicyclo[5.4.0]undec- | | |
| | 7-ene | | |
| DCC | N,N′- | TNP- | 2′,3′-O-Trinitrophenyl- |
| | Dicyclohexylcarbodiimide | ATP | adenosine-5′-triphosphate |
| DCE | Dichloroethane | Ph | Phenyl |
| DME | Dimethoxyethane | Rt | Room temperature |
| Et | Ethyl | RZ | Robust Z prime factor |
| EtOAc | Ethyl acetate | S.D. | Standard deviation |
| Eq | equivalents | TBAF | Tetra-n-butylammonium |
| | | | fluoride |
| h | Hour | t-Bu | Tert-butyl |
| hetAr | Heteroaryl | TEA | Triethylamine |
| Kg | Kilogram | Tf₂O | Triflic anhydride |
| MCAo | Middle cerebral artery | THF | Tetrahydrofuran |
| | occlusion | | |
| Me | Methyl | thio- | thiocarbonydiimidazole |
| | | CDI | |
| MeOH | Methanol | TMS | Trimethylsilyl |
| Mg | milligram | | |

The following terms are used herein.

“Acute phase” as used herein means the time period starting at the time a subject has a stroke and lasting from the time of stroke to day 7 after stroke. In humans, the acute phase is somewhat variable, but generally, human subjects are hospitalized during the acute phase of stroke.

“Subacute phase” as used herein means the time period from 7 days to about 3 months after a subject has a stroke. This is the phase in which human subjects experience the most recovery.

“Chronic phase” as used herein means the time period comprising about 3 months after stroke to end of life. In humans, substantial progress can be made during the chronic phase of stroke.

A dash (“-”) that is not between two letters or symbols is used to indicate a point of attachment for a substituent. For example, —COOH is attached through the carbon atom.

“Alkyl” as used herein means branched and straight-chain saturated aliphatic hydrocarbon groups having the specified number of carbon atoms. Thus, the term C₁-C₆ alkyl as used herein includes alkyl groups having from 1 to about 6 carbon atoms. When C₀-Cₙ alkyl is used herein in conjunction with another group, for example, phenylC₀-C₄ alkyl, the indicated group, in this case phenyl, is either directly bound by a single covalent bond (C₀), or attached by an alkyl chain having the specified number of carbon atoms, in this case from 1 to 4 carbon atoms. Examples of alkyl groups include, but are not limited to, methyl, ethyl, n-propyl, isopropyl, n-butyl, t-butyl, n-pentyl, and sec-pentyl.

“Alkoxy” as used herein means an alkyl group as defined above with the indicated number of carbon atoms attached through an oxygen bridge. Examples of alkoxy groups include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy.

“Alkanoyl” as used herein means an alkyl group as defined above, attached through a keto (—(C═O)—) bridge. Alkanoyl groups have the indicated number of carbon atoms, with the carbon of the keto group being included in the numbered carbon atoms. For example, a C₂alkanoyl group is an acetyl group having the formula CH₃(C═O)—.

“Aryl” as used herein means an aromatic group containing only carbon in the aromatic ring or rings. Such aromatic groups can be further substituted with carbon or non-carbon atoms or groups. Aryl groups can have 1 to 3 separate, fused, or pendant rings without heteroatoms as ring members. Substitution can include fusion to a 5 to 7-membered saturated cyclic group that optionally contains 1 or 2 heteroatoms independently chosen from N, O, and S, to form, for example, a 3,4-methylenedioxy-phenyl group. Examples of aryl groups include, but are not limited to, phenyl, naphthyl (including 1-naphthyl and 2-naphthyl), and bi-phenyl.

“(Aryl)alkyl” as used herein means a group including an aryl group and an alkyl group as defined above, where the point of attachment of the group is via the alkyl moiety. Examples of (aryl)alkyl group include, but are not limited to, benzyl, phenylethyl, and piperonyl.

“Cycloalkyl” as used herein means a saturated hydrocarbon ring group having the specified number of carbon atoms. Examples of cycloalkyl groups include, but are not limited to, cyclopropyl, cyclobutyl, cyclopentyl, and cyclohexyl as well as bridged or caged saturated ring groups such as norbornane or adamantane.

“Haloalkyl” as used herein means branched and straight-chain saturated aliphatic alkyl group as defined above having the specified number of carbon atoms and substituted with 1 or more halogen atoms, for example up to the maximum allowable number of halogen atoms. Examples of haloalkyl groups include, but are not limited to, trifluoromethyl, difluoromethyl, 2-fluoroethyl, and penta-fluoroethyl.

“Haloalkoxy” as used herein means a haloalkyl group as defined above attached through an oxygen bridge.

“Halo” or “halogen” as used herein means fluoro, chloro, bromo, or iodo. A combination of different halogen groups can be present, for example a chlorofluoromethyl group.

“Heteroaryl” as used herein means an aromatic ring group having the specified number of carbon atoms and at least 1, preferably 1 to 4 heteroatoms in the ring, where the heteroatoms can each independently be N, O, S, Si, or P. In an aspect, a heteroaryl group is a stable 5- to 7-membered monocyclic or 7- to 10-membered bicyclic heterocyclic ring group where at least 1 aromatic ring contains from 1 to 4, or from 1 to 3, heteroatoms that can each independently be N, O, or S, with the remaining ring atoms being carbon. When the total number of S and O atoms in the heteroaryl group exceeds 1, these heteroatoms are not adjacent to one another. Preferably, the total number of S and O atoms in the heteroaryl group is 1 or 2. Examples of heteroaryl groups include, but are not limited to, pyridyl, indolyl, pyrimidinyl, pyridizinyl, pyrazinyl, imidazolyl, oxazolyl, furanyl, thiophenyl, thiazolyl, triazolyl, tetrazolyl, isoxazolyl, quinolinyl, pyrrolyl, pyrazolyl, and 5,6,7,8-tetrahydroisoquinoline.

“Heteroarylalkyl” as used herein means a group having the indicated number of carbon atoms and including a heteroaryl group and an alkyl group as defined above where the point of attachment of the group is via the alkyl moiety. This term includes, but is not limited to, pyridylmethyl, thiophenylmethyl, and pyrrolyl(1-ethyl).

“Heterocycloalkyl” as used herein means a saturated cyclic ring group having the indicated number of carbon atoms and from 1 to 3 heteroatoms in the ring, wherein the heteroatoms can be N, O, or S. In an aspect, heterocycloalkyl groups have from 3 to 8 ring atoms or 5 to 7 ring atoms and 1, 2, or 3 heteroatoms that can each independently be N, O, or S. Examples of heterocycloalkyl groups include, but are not limited to, morpholinyl, piperazinyl, piperidinyl, pyrrolidinyl, 1,2,4-oxadiazol-3-yl-5(4H)-thione, and 1,2,4-oxadiazol-3-yl-5(4H)-one groups.

“Pharmaceutically acceptable salt” as used herein means a derivative of a compound wherein the parent compound is modified by making an acid or base salt thereof, and further includes pharmaceutically acceptable solvates of such compounds and such salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional salts and the quaternary ammonium salts of the parent compound formed, for example, from inorganic or organic acids. For example, conventional acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Pharmaceutically acceptable salts can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable.

The term “substituted” as used herein means that any one or more hydrogens on the designated atom or group is replaced with a selection from the indicated group, provided that the designated atom's normal valence is not exceeded. When a substituent is oxo (i.e., ═O), then 2 hydrogens on the atom are replaced. When aromatic moieties re substituted by an oxo group, the aromatic ring is replaced by the corresponding partially unsaturated ring. For example, a pyridyl group substituted by oxo is a pyridone. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent. A hydrogen substituent is a hydrogen atom. The number of carbon atoms in a given group does not include any substituents. For example, a 3-cyanophenyl group is a C₆ aryl group.

A phenyl, pyridyl, imidazolyl, oxazolyl, or thiazolyl group can be optionally substituted with a substituent that is a C₁₋₈ alkyl, C₂₋₈ alkenyl, C₁₋₈ alkoxy, C₁₋₈ alkyl group substituted with 1 to 3 halogen atoms, C₁₋₈ alkoxy substituted with 1 to 3 halogen atoms, a halogen atom, hydroxyl, nitro, cyano, amino, C₁₋₈ alkylamino, C₂₋₈ dialkylamino, or an aralkyl group, as a substituent.

The terms “a” and “an” and “the” and similar referents (especially in the context of the following claims) are to be construed to cover both the singular and the plural, unless otherwise indicated herein or clearly contradicted by context. “Or” means “and/or”. The terms first, second, third, etc. as used herein do denote any particular ordering, but simply for convenience to denote a plurality of, for example, steps. The terms “comprising”, “having”, “including”, “containing”, and various thereof are to be construed as open-ended terms (i.e., meaning “including, but not limited to”) unless otherwise noted. Recitation of ranges of values are serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to exemplary aspects, it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular aspect disclosed as the best mode contemplated for carrying out this invention, but that the invention will include all aspects falling within the scope of the appended claims. Any combination of the above-described elements in all possible variations thereof is encompassed by the invention unless otherwise indicated herein or otherwise clearly contradicted by context.

1. A method for treatment of a human subject who has had a stroke or myocardial ischemia reperfusion injury, the method comprising:
administering to the subject a pharmaceutical composition comprising a compound of Formula (I) 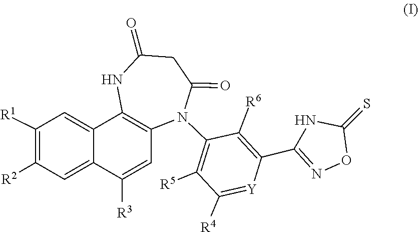
or a pharmaceutically acceptable salt thereof, wherein in Formula (I),
R¹ is hydrogen, cyano, halo, nitro, C₁-C₃ alkyl, or C₁-C₃ haloalkyl,
R² is hydrogen, cyano, halo, nitro, C₁-C₆ alkyl, C₁-C₆ haloalkyl, C₁-C₆ alkoxy, C₁-C₆ haloalkoxy, C₃—C cycloalkyl, C₃-C₈ cycloalkoxy, C₂-C₇ heterocycloalkyl, C₆-C₁₂ aryl, C₂-C₁₁ heteroaryl, C₂-C₆ alkanoyl, —COOH, —NR^{a}R^{b}, —C(O)—OR^{c}, —C(O)—NR^{a}R^{b}, —SO₂—OR^{c} or —SO₂—NR^{a}R^{b}, wherein R^{a}, R^{b}, and R^{c} are independently hydrogen, C₁-C₆ alkyl, or C₁-C₆ haloalkyl,
R³ is hydrogen, cyano, halo, nitro, C₁-C₆ alkyl, C₁-C₆ haloalkyl, C₁-C₆ alkoxy, C₁-C₆ haloalkoxy, C₃-C₈ cycloalkyl, C₃-C₈ cycloalkoxy, C₂-C₇ heterocycloalkyl, C₆-C₁₂ aryl, C₂-C₁₁ heteroaryl, C₂-C₆ alkanoyl, —COOH, —NR^{a}R^{b}, —C(O)—OR^{a}, —C(O)—NR^{a}R^{b}, —SO₂—OR^{a} or —SO₂—NR^{a}R^{b}, wherein R^{a} and R^{b}, and R⁶ are each independently hydrogen, C₁-C₆ alkyl, or C₁-C₆ haloalkyl,
R⁴, R⁵, and R⁶ are each independently hydrogen, cyano, halo, nitro, C₁-C₆ alkyl, C₁-C₆ haloalkyl, C₁-C₆ alkoxy, C₁-C₆ haloalkoxy, C₃-C₈ cycloalkyl, C₃-C₈ cycloalkoxy, C₂-C₇ heterocycloalkyl, C₆-C₁₂ aryl, C₂-C₁₁ heteroaryl, C₂-C₆ alkanoyl, —COOH, —NR^{a}R^{b}, —C(O)—OR^{c}, —C(O)—NR^{a}R^{b}, —SO₂—OR^{c} or —SO₂—NR^{a}R^{c},
with the proviso that not all of R¹, R², and R³ are hydrogen; and
Y is CH, CR⁴, or N.

2. The method of claim 1, wherein
R¹ is hydrogen, cyano, halo, methyl, or halomethyl,
R² is hydrogen, cyano, halo, C₁-C₆ alkyl, C₁-C₆ haloalkyl, C₁-C₆ alkoxy, C₁-C₆ haloalkoxy, C₃-C₆ cycloalkyl, C₃-C₆ cycloalkoxy, C₂-C₅ heterocycloalkyl, C₆ aryl, C₂-C₆ heteroaryl, C₂-C₆ alkanoyl, —COOH, —NR^{a}R^{b}, —C(O)—OR^{a}, —C(O)—NR^{b}R⁶, —SO₂—OR⁷ or —SO₂—NR^{b}R⁶, wherein R^{a}, R^{b}, and R⁶ are each independently hydrogen, C₁-C₃ alkyl, or C₁-C₃ haloalkyl,
R³ is hydrogen, C₁-C₆ alkyl, C₁-C₆ haloalkyl, C₁-C₆ alkoxy, C₁-C₆ haloalkoxy, C₃-C₆ cycloalkyl, C₃-C₆ cycloalkoxy, C₂-C₅ heterocycloalkyl, C₆ aryl, C₂-C₆ heteroaryl, C₂-C₆ alkanoyl, —COOH, —NR^{a}R^{b}, —C(O)—OR^{c}, —C(O)—NR^{a}R^{b}, —SO₂—OR^{c} or —SO₂—NR^{a}R^{b}, wherein R^{a}, R^{b}, and R^{c} are each independently hydrogen, C₁-C₃ alkyl, or C₁-C₃ haloalkyl,
R⁴, R⁵, and R⁶ are each independently hydrogen, C₁-C₆ alkyl, C₁-C₆ haloalkyl, C₁-C₆ alkoxy, C₁-C₆ haloalkoxy, C₃-C₆ cycloalkyl, C₃-C₆ cycloalkoxy, C₂-C₅ heterocycloalkyl, C₆ aryl, C₂-C₆ heteroaryl, C₂-C₆ alkanoyl, —COOH, —NR^{a}R^{b}, —C(O)—OR^{c}, —C(O)—NR^{a}R^{b}, —SO₂—OR^{c} or —SO₂—NR^{a}R^{b},
with the proviso that not all of R¹, R², and R³ are hydrogen; and
Y is CH, CR⁴, or N.

3. The method of claim 1, wherein
R¹ is hydrogen,
R² is hydrogen, halo, or C₁-C₃ alkyl,
R³ is hydrogen, halo, or C₁-C₃ alkyl,
R⁴ is hydrogen, halo, or C₁-C₃ alkyl,
R⁵ and R⁶ are each hydrogen,
with the proviso that not all of R1, R2, and R3 are hydrogen; and
Y is CH, CR4, or N.

4. The method of claim 1, wherein the compound of Formula (I) or the pharmaceutically acceptable salt and/or formulation thereof is a compound of the following formulas, a pharmaceutically acceptable salt thereof, or the indicated salt form thereof.

| Compound | Structure | Salt form |
|---|---|---|
| | 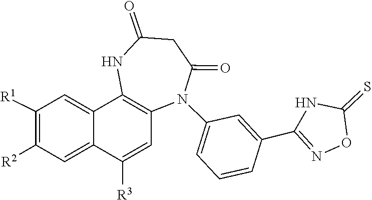 | |
| 22b | R¹ = CH₃, R² = R³ = H | Sodium salt |
| 22c^{d} | R¹ = H, R² = CH3, R³ = H | Sodium salt |
| 21d^{d} | R¹ = R² = H, R³ = Br | Triethylamine salt |
| 21e | R¹ = CH₃, R² = H, R³ = Br | Triethylamine salt |
| 21j | R¹ = H, R² = CH₂N₃, R³ = H | Triethylamine salt |
| 211 | R¹ = R² = H, R³ = Ph | Triethylamine salt |
| 21p | R¹ = R² = H, R³ = Et | Triethylamine salt |
| | 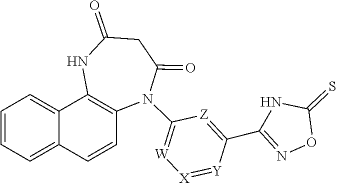 | |
| 21r | X = CCH₃, W = Y = Z = CH | Triethylamine salt |
| 21s | W = N, X = Y = Z = CH | Triethylamine salt |
| 21t | X = N, W = Y = Z = CH | Triethylamine salt |
| 21u^{d} | Y = N, W = X = Z = CH | Triethylamine salt |
| 21v | Z = N, W = X = Y = CH | Triethylamine salt |

5. The method of claim 1, wherein in the compound of Formula (I) or the pharmaceutically acceptable salt and/or formulation thereof, is a compound of the formulas (Ia), (Ib), or (Ic)

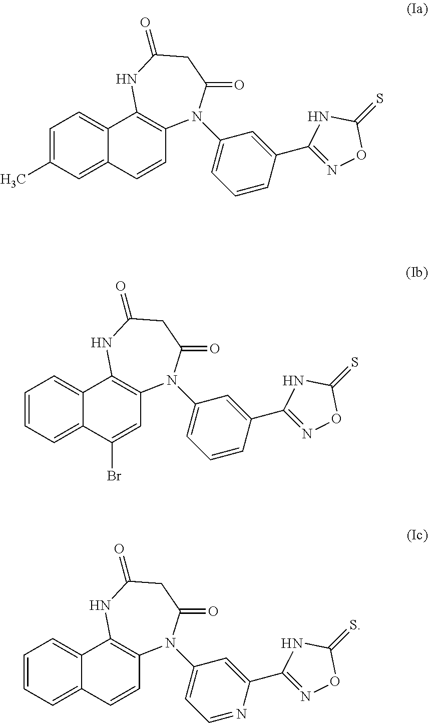 [SMILES: Cc1ccc2c3c(ccc2c1)N(c1cccc(-c2noc(=S)[nH]2)c1)C(=O)CC(=O)N3 ; O=C1CC(=O)N(c2cccc(-c3noc(=S)[nH]3)c2)c2cc(Br)c3ccccc3c2N1 ; O=C1CC(=O)N(c2ccnc(-c3noc(=S)[nH]3)c2)c2ccc3ccccc3c2N1]

6. The method of claim 1, wherein the compound of Formula (I) is an antagonist of the P2X4 receptor.

7. The method of claim 1, wherein compound of Formula (I) is administered at a dosage of about 0.05 mg/kg to about 0.5 mg/kg to about 5 mg/kg of body weight of the subject.

8. The method of claim 1, wherein the administering pharmaceutical composition is by oral administration or intravenous injection.

9. The method of claim 1, wherein the human subject has had a stroke, and the stroke is an ischemic stroke.

10. The method of claim 1, wherein the human subject has had a stroke, and the pharmaceutical composition is administered during the acute phase of the stroke, wherein the acute phase of stroke starts at the time the stroke occurs and lasting for 7 days.

11. The method of claim 9, wherein the administration of the pharmaceutical composition is ceased after the day 7.

12. The method of claim 9, wherein the administration of the pharmaceutical composition is continued through the subacute and/or the chronic phase of stroke.

13. The method of claim 1, wherein the human subject has had a stroke, and intravenous injection is injection into the general circulation, or targeted infusion whereby pharmaceutical composition is supplied close to the site of the blockage that triggered the stroke.

14. The method of claim 13, wherein the infusion is provided by an endovascular catheter, wherein the endovascular catheter has been previously used to provide a thrombolytic therapeutic agent to the subject, or the endovascular catheter has been previously used in conjunction with a procedure on the subject involving a clot-removal device.

15. (canceled)

16. (canceled)

17. The method of claim 1, wherein the human subject has had a stroke, and
the pharmaceutical composition is administered from one minute to up to 3 hours before to administering a thrombolytic therapeutic or applying a clot-removal device to the subject; or
the pharmaceutical composition is administered concomitantly with a thrombolytic therapeutic or applying a clot-removal device to the subject; or
the pharmaceutical composition is administered after a thrombolytic therapeutic or clot-removal device is administered to the subject.

18. The method of claim 1, wherein the human subject has had a myocardial ischemia reperfusion injury as a result of myocardial infarction.

19. The method of claim 18 wherein the compound is of the formula:

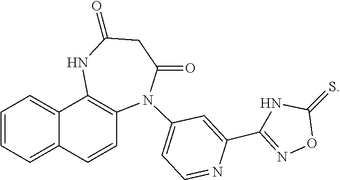 [SMILES: O=C1CC(=O)N(c2ccnc(-c3noc(=S)[nH]3)c2)c2ccc3ccccc3c2N1]

20. The method of claim 18 wherein the development of cardiac dysfunction is treated.

21. A method for treatment of a human subject who has had a stroke or myocardial ischemia reperfusion injury, the method comprising:
administering to the subject a pharmaceutical composition comprising a compound of Formula (5) 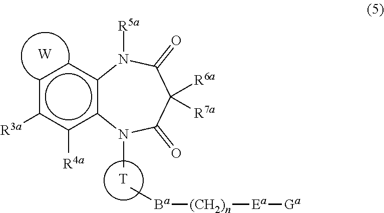
or a pharmaceutically acceptable salt thereof, wherein, in Formula (5),
the moiety 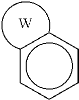
is naphthalene ring, quinoline ring, isoquinoline ring, tetrahydronaphthalene ring, indane ring, tetrahydroquinoline ring, or tetrahydroisoquinoline ring, each ring optionally substituted with 1 to 4 substituents that are the same or different and are C₁₋₈ alkyl, C₂₋₈ alkenyl, C₁₋₈ alkoxy, C₁₋₈ alkyl substituted with 1 to 3 halogen atoms, C₁₋₈ alkoxy substituted with 1 to 3 halogen atoms, a halogen atom, hydroxyl, nitro, cyano, amino, C₁₋₈ alkylamino, C₂₋₈ dialkylamino, C₂₋₈ acylamino, carboxyl, C₂₋₈ acyl, an alkoxycarbonyl (wherein the alkoxy moiety has 1 to 8 carbon atoms), or an aralkyl (wherein the aryl moiety has 6 to 10 carbon atoms, and the alkylene moiety has 1 to 8 carbon atoms),
R^{3a} and R^{4b} are the same or different, and are a hydrogen atom, C₁₋₈ alkyl, C₂₋₈ alkenyl, C₁₋₈ alkoxy, C₁₋₈ alkyl substituted with 1 to 3 halogen atoms, C₁₋₈ alkoxy substituted with 1 to 3 halogen atoms, a halogen atom, hydroxyl, nitro, cyano, amino, C₁₋₈ alkylamino, C₂₋₈ dialkylamino, C₂₋₈ acylamino, carboxyl, C₂₋₈ acyl, an alkoxycarbonyl group (wherein the alkoxy moiety has 1 to 8 carbon atoms), or an aralkyl group (wherein the aryl moiety has 6 to 10 carbon atoms, and the alkylene moiety has 1 to 8 carbon atoms),
R^{5a} is a hydrogen atom, C₁₋₈ alkyl, C₂₋₈ alkenyl, C₁₋₈ alkyl substituted with 1 to 3 halogen atoms, C₁₋₈ alkyl substituted with a hydroxyl, or an aralkyl (wherein the aryl moiety has 6 to 10 carbon atoms, and the alkylene moiety has 1 to 8 carbon atoms),
R^{6a} and R^{7a} are the same or different, and represent a hydrogen atom, C₁₋₈ alkyl, C₂₋₈ alkenyl, C₁₋₈ alkoxy, C₁₋₈ alkyl substituted with 1 to 3 halogen atoms, C₁₋₈ alkoxy substituted with 1 to 3 halogen atoms, a halogen atom, hydroxyl, or amino,
the moiety 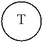
is a benzene ring, pyridine ring, thiophene ring, pyrimidine ring, naphthalene ring, quinoline ring, or indole ring, which can optionally have 1 to 4 of the same or different substituents that can be C₁₋₈ alkyl, C₂₋₈ alkenyl, C₁₋₈ alkoxy, C₁₋₈ alkyl substituted with 1 to 3 halogen atoms, C₁₋₈ alkoxy substituted with 1 to 3 halogen atoms, a halogen atom, hydroxyl, nitro, cyano, amino, C₁₋₈ alkylamino, C₂₋₈ dialkylamino, an aralkyl group (wherein the aryl moiety has 6 to 10 carbon atoms, and the alkylene moiety has 1 to 8 carbon atoms), phenyl, and pyridyl, as a substituent,
B^{a} is N(R^{8a})C(═O), NHCONH, CON(R^{9a}), NHC(═S)NH, N(R^{10a})SO₂, SO₂N(R^{11a}), or OSO₂, wherein R^{8a}, R^{9a}, R^{10a} and R¹¹ are hydrogen, C₁₋₈ alkyl, C₁₋₈ alkyl substituted with 1 to 3 halogen atoms, C₁₋₈ alkyl substituted with a hydroxyl, or an aralkyl wherein the aryl moiety has 6 to 10 carbon atoms, and the alkylene moiety has 1 to 8 carbon atoms,
E^{a} is O, S, NR^{12a}, or an atomic bond, wherein R^{12a} is a hydrogen, C₁₋₈ alkyl, C₂₋₈ alkenyl, a C₁₋₈ alkyl substituted with 1 to 3 halogen atoms, C₁₋₈ alkyl substituted with a hydroxyl, or an aralkyl (wherein the aryl moiety has 6 to 10 carbon atoms, and the alkylene moiety has 1 to 8 carbon atoms),
G^{a} is piperazine, piperidine, morpholine, cyclohexane, benzene, naphthalene, quinoline, quinoxaline, benzimidazole, thiophene, imidazole, thiazole, oxazole, indole, benzofuran, pyrrole, pyridine, or pyrimidine, which can be optionally substituted with 1 to 4 of the same or different substituents that can be C₁₋₈ alkyl, C₂₋₈ alkenyl, C₁₋₈ alkoxy, C₁₋₈ alkyl substituted with 1 to 3 halogen atoms, C₁₋₈ alkoxy substituted with 1 to 3 halogen atoms, a halogen atom, hydroxyl, nitro, cyano, amino, C₁₋₈ alkylamino, C₂₋₈ dialkylamino, C₂₋₈ acyl, methylenedioxy, carboxyl, C₁₋₆ alkylsulfinyl, C₁₋₆ alkylthio, C₁₋₆ alkylsulfonyl, an aralkyl (wherein the aryl moiety has 6 to 10 carbon atoms, and the alkylene moiety has 1 to 8 carbon atoms), an optionally substituted phenyl, an optionally substituted pyridyl, an optionally substituted imidazolyl, an optionally substituted oxazolyl, or an optionally substituted thiazolyl, as a substituent, and
n is an integer of 0 to 5.

22. The method of claim 21, wherein the compound of Formula (5) or the pharmaceutically acceptable salt and/or formulation thereof is a compound of Formula A to Formula LLL of Table 2 or a compound of Formula (5a)

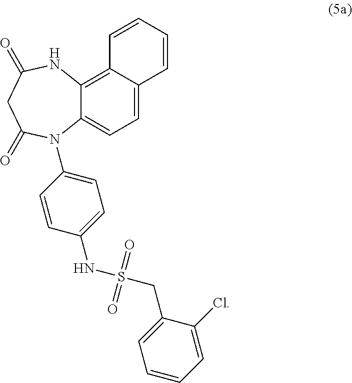 [SMILES: O=C1CC(=O)N(c2ccc(NS(=O)(=O)Cc3ccccc3Cl)cc2)c2ccc3ccccc3c2N1]

23-37. (canceled)
